(19) United States
(12) Patent Application Publication
(10) Pub. No.: US 2024/0327556 A1
(43) Pub. Date: Oct. 3, 2024

(54) COMPOUND, POLYMERIZABLE COMPOSITION, OPTICALLY ANISOTROPIC FILM, OPTICAL FILM, POLARIZING PLATE, AND IMAGE DISPLAY DEVICE

(71) Applicant: FUJIFILM Corporation, Tokyo (JP)

(72) Inventors: Shinichi MORISHIMA, Kanagawa (JP); Kouitsu SASAKI, Kanagawa (JP); Ryoji HIMENO, Kanagawa (JP); Takafumi NAKAYAMA, Kanagawa (JP); Naozumi SHIRAIWA, Kanagawa (JP); Mayumi NOJIRI, Kanagawa (JP); Takashi KATOU, Kanagawa (JP)

(73) Assignee: FUJIFILM Corporation, Tokyo (JP)

(21) Appl. No.: 18/741,242

(22) Filed: Jun. 12, 2024

Related U.S. Application Data
(63) Continuation of application No. PCT/JP2022/044542, filed on Dec. 2, 2022.

(30) Foreign Application Priority Data
Dec. 15, 2021 (JP) 2021-203523

(51) Int. Cl. C08F 222/20; C07C 69/753; C07C 243/20; C07C 255/55; C07D 209/12; C07D 231/36; C07D 233/86; C07D 239/62; C07D 241/38; C07D 277/10; C07D 277/82; C07D 339/02; C07D 339/06; C07D 409/14; C07D 495/04; C08J 5/18; C09K 19/38; G02B 1/04; G02B 5/30

(52) U.S. Cl. CPC C08F 222/20; C07C 69/753; C07C 243/20; C07C 255/55; C07D 209/12; C07D 231/36; C07D 233/86; C07D 239/62; C07D 241/38; C07D 277/10; C07D 277/82; C07D 339/02; C07D 339/06; C07D 409/14; C07D 495/04; C08J 5/18; C09K 19/3861; G02B 1/04; C07C 2602/08; C08J 2335/02; C09K 2219/03; G02B 5/3025

(57) ABSTRACT

A compound that is suitably used for formation of an optically anisotropic film having excellent reverse wavelength dispersibility and moisture-heat resistance; a polymerizable composition; an optically anisotropic film; an optical film; a polarizing plate; and an image display device. The present invention relates to a compound represented by Formula (I-1) P¹-SP¹-Ar¹-Mes-Ar²-SP²-P² (I-1).

### CROSS-REFERENCE TO RELATED APPLICATIONS

This application is a Continuation of PCT International Application No. PCT/JP2022/044542 filed on Dec. 2, 2022, which was published under PCT Article 21 (2) in Japanese, and which claims priority under 35 U.S.C. § 119 (a) to Japanese Patent Application No. 2021-203523 filed on Dec. 15, 2021. The above applications are hereby expressly incorporated by reference, in their entirety, into the present application.

### BACKGROUND OF THE INVENTION

### 1. Field of the Invention

The present invention relates to a compound, polymerizable composition, an optically anisotropic film, an optical film, a polarizing plate, and an image display device.

### 2. Description of the Related Art

A compound exhibiting reverse wavelength dispersibility enables, for example, accurate conversion of ray wavelengths over a wide wavelength range and reduction in the thickness of a phase difference film due to its high refractive index, and therefore, it has been actively studied.

In addition, for the polymerizable liquid crystal compound exhibiting reverse wavelength dispersibility, T-type molecular design guidelines have generally been adapted, and thus, it has been required to decrease the wavelength of the molecular major axis and increase the wavelength of the minor axis positioned at the center of the molecule.

Furthermore, in order to increase the reverse wavelength dispersibility with a T-type molecular design, a polymerizable liquid crystal compound in which two structures with an increased wavelength are introduced in a central portion of the molecule is known (see, for example, JP2020-134634A and WO2018/123586A).

### SUMMARY OF THE INVENTION

The present inventors have conducted studies on JP2020-134634A and WO2018/123586A, and have found that the reverse wavelength dispersibility of an optically anisotropic film thus formed is insufficient and the formed optically anisotropic film has a change in birefringence index (that is, a deterioration in moisture-heat resistance) in a case where it is exposed under a high temperature and a high humidity.

Therefore, an object of the present invention is to provide a compound that is suitably used for formation of an optically anisotropic film having excellent reverse wavelength dispersibility and moisture-heat resistance; a polymerizable composition; an optically anisotropic film; an optical film; a polarizing plate; and an image display device.

The present inventors have conducted intensive studies to accomplish the object, and as a result, they have found that the reverse wavelength dispersibility and the moisture-heat resistance of an optically anisotropic film thus formed are improved by using a compound represented by Formula (I-1) which will be described later, thereby completing the present invention.

That is, the present inventors have found that the object can be accomplished by the following configurations.

[1] A compound represented by Formula (I-1) which will be described later.

[2] A polymerizable composition comprising:
the compound represented by Formula (I-1) which will be described later; and
a liquid crystal compound represented by Formula (II) which will be described later,
in which a content of the compound represented by Formula (I-1) which will be described later is 10% by mass or more with respect to a total mass of the compound represented by Formula (I-1) which will be described later and the liquid crystal compound represented by Formula (II) which will be described later.

[3] An optically anisotropic film obtained by polymerizing the polymerizable composition as described in [2].

[4] An optical film comprising:
the optically anisotropic film as described in [3].

[5] A polarizing plate comprising:
the optical film as described in [4]; and
a polarizer.

[6] An image display device comprising:
the optical film as described in [4] or the polarizing plate as described in [5].

According to the present invention, it is possible to provide a compound that is suitably used for formation of an optically anisotropic film having excellent reverse wavelength dispersibility and moisture-heat resistance; a polymerizable composition; an optically anisotropic film; an optical film; a polarizing plate; and an image display device.

### BRIEF DESCRIPTION OF THE DRAWINGS

FIG. 1A is a schematic cross-sectional view showing an example of an optical film of an embodiment of the present invention.

FIG. 1B is a schematic cross-sectional view showing an example of the optical film of the embodiment of the present invention.

FIG. 1C is a schematic cross-sectional view showing an example of the optical film of the embodiment of the present invention.

### DESCRIPTION OF THE PREFERRED EMBODIMENTS

Hereinafter, the present invention will be described in detail.

Descriptions on the constitution requirements which will be described later are made based on representative embodiments of the present invention in some cases, but it should not be construed that the present invention is limited to such embodiments.

Furthermore, in the present specification, a numerical range expressed using “to” means a range including numerical values before and after “to” as a lower limit value and an upper limit value.

In addition, in the present specification, for each component, one kind of substance corresponding to each component may be used alone, or two or more kinds thereof may be used in combination. Here, in a case where the two or more kinds of the substances are used in combination for each component, the content of the component refers to a total content of the substances used in combination unless otherwise specified.

In addition, in the present specification, an expression, “may have a substituent”, includes not only one aspect of not having a substituent but also one aspect of having one or more substituents.

Here, examples of the substituent include a substituent E described below.

### Substituent E

Examples of the substituent E include an alkyl group, an alkoxy group, an alkylcarbonyl group, an alkoxycarbonyl group, an alkylcarbonyloxy group, an alkylamino group, a dialkylamino group, an alkylamide group, an alkenyl group, an alkynyl group, a halogen atom, a cyano group, a nitro group, an alkylthiol group, and an N-alkylcarbamate group, and among these, the alkyl group, the alkoxy group, the alkoxycarbonyl group, the alkylcarbonyloxy group, or the halogen atom is preferable.

As the alkyl group, a linear, branched, or cyclic alkyl group having 1 to 18 carbon atoms is preferable, an alkyl group having 1 to 8 carbon atoms (for example, a methyl group, an ethyl group, a propyl group, an isopropyl group, an n-butyl group, an isobutyl group, a sec-butyl group, a t-butyl group, and a cyclohexyl group) is more preferable, an alkyl group having 1 to 4 carbon atoms is still more preferable, and the methyl group or the ethyl group is particularly preferable.

As the alkoxy group, an alkoxy group having 1 to 18 carbon atoms is preferable, an alkoxy group having 1 to 8 carbon atoms (for example, a methoxy group, an ethoxy group, an n-butoxy group, and a methoxyethoxy group) is more preferable, an alkoxy group having 1 to 4 carbon atoms is still more preferable, and the methoxy group or the ethoxy group is particularly preferable.

Examples of the alkoxycarbonyl group include a group in which an oxycarbonyl group (—O—CO— group) is bonded to the alkyl group exemplified above, and among these, a methoxycarbonyl group, an ethoxycarbonyl group, an n-propoxycarbonyl group, or an isopropoxycarbonyl group is preferable, and the methoxycarbonyl group is more preferable.

Examples of the alkylcarbonyloxy group include a group in which a carbonyloxy group (—CO—O— group) is bonded to the alkyl group exemplified above, and among these, a methylcarbonyloxy group, an ethylcarbonyloxy group, an n-propylcarbonyloxy group, or an isopropylcarbonyloxy group is preferable, and the methylcarbonyloxy group is more preferable.

Examples of the halogen atom include a fluorine atom, a chlorine atom, a bromine atom, and an iodine atom, and among these, the fluorine atom or the chlorine atom is preferable.

### Compound

The compound of an embodiment of the present invention is a compound represented by Formula (I-1) which will be described later (hereinafter also simply referred to as a “specific compound”).

In the present invention, the reverse wavelength dispersibility and the moisture-heat resistance of an optically anisotropic film thus formed are improved by using the specific compound, as described above.

A reason therefor is not specifically clear, but is presumed to be as follows by the present inventors.

That is, it is considered that since in Formula (I-1) which will be described later, linking groups (SP¹ and SP²) adjacent to predetermined ring structures (Ar¹ and Ar²) do not include an aromatic ring or a cyclohexane ring having a large steric hindrance, an energy-stable structure without a distortion of the ring structures (Ar¹ and Ar²) can be adopted, and as a result, the hydrolysis resistance and the moisture-heat resistance are improved.

In addition, in Formula (I-1) which will be described later, it is considered that since a π-type molecular design is adopted unlike a T-type molecular design in the related art, the planar aligning properties are improved, and as a result, high reverse wavelength dispersibility due to the π-type structure is exhibited.

Hereinafter, the specific compound of the embodiment of the present invention will be described in detail.

### Specific Compound

The specific compound is a compound represented by Formula (I-1) which will be described later.

P¹-SP¹-Ar¹-Mes-Ar²-SP²-P² (I-1)

In Formula (I-1), P¹ and P² each independently represent a monovalent organic group.

Examples of the monovalent organic group represented by each of P¹ and P² include an alkyl group, an aryl group, and a heteroaryl group.

The alkyl group may be linear, branched, or cyclic, but is preferably linear. The number of carbon atoms of the alkyl group is preferably 1 to 30, more preferably 1 to 20, and still more preferably 1 to 10.

In addition, the aryl group may be a monocycle or a polycycle, but is preferably the monocycle. The number of carbon atoms of the aryl group is preferably 6 to 25, and more preferably 6 to 10.

In addition, the heteroaryl group may be a monocycle or a polycycle. The number of heteroatoms constituting the heteroaryl group is preferably 1 to 3. The heteroatom constituting the heteroaryl group is preferably a nitrogen atom, a sulfur atom, or an oxygen atom. The number of carbon atoms of the heteroaryl group is preferably 6 to 18, and more preferably 6 to 12.

In addition, the alkyl group, the aryl group, and the heteroaryl group may be unsubstituted or have a substituent. Examples of the substituent include the above-described substituent E.

In the present invention, it is preferable that at least one of P¹ or P² represents a polymerizable group.

Here, the polymerizable group is not particularly limited, and a radically polymerizable group or a cationically polymerizable group is preferable.

A known radically polymerizable group can be used as the radically polymerizable group, and suitable examples thereof include an acryloyloxy group or a methacryloyloxy group. In this case, it is known that the acryloyloxy group generally has a high polymerization rate, and from the viewpoint of improvement of productivity, the acryloyloxy group is preferable but the methacryloyloxy group can also be used as the polymerizable group.

A known cationically polymerizable group can be used as the cationically polymerizable group, and specific examples thereof include an alicyclic ether group, a cyclic acetal group, a cyclic lactone group, a cyclic thioether group, a spiroorthoester group, and a vinyloxy group. Among those, the alicyclic ether group or the vinyloxy group is suitable, and an epoxy group, an oxetanyl group, or the vinyloxy group is particularly preferable.

Particularly preferred examples of the polymerizable group include a polymerizable group represented by any of Formulae (P-1) to (P-20).

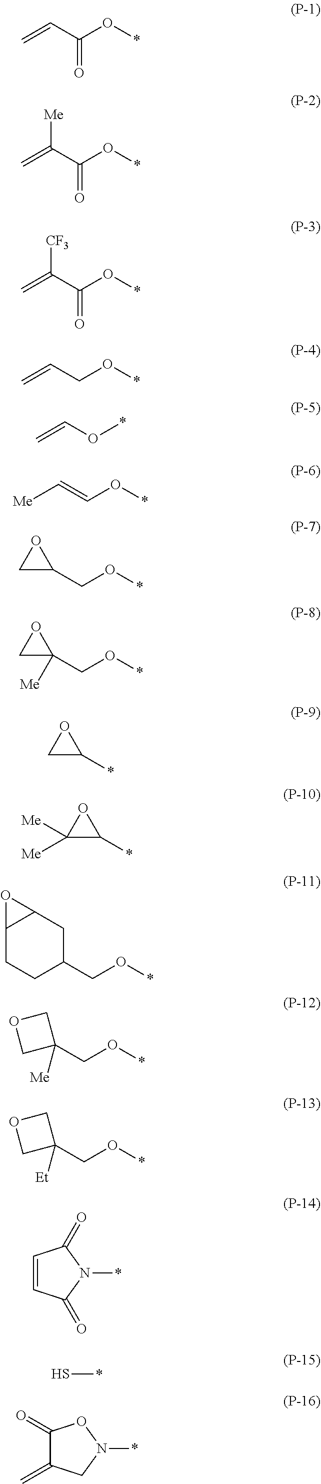

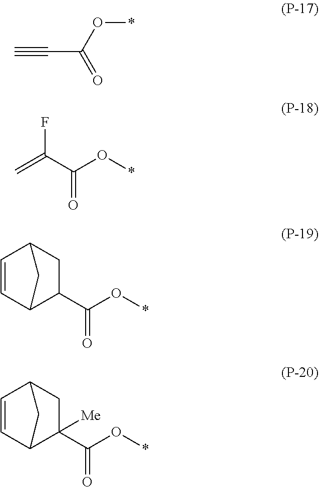

For a reason that the moisture-heat resistance of an optically anisotropic film thus formed is further improved, either of P¹ and P² is preferably a polymerizable group, and more preferably an acryloyloxy group or a methacryloyloxy group.

In Formula (I-1), SP¹ and SP² each independently represent a linear or branched alkylene group having 1 to 12 carbon atoms, or a divalent linking group in which one or more of —CH₂—'s constituting the linear or branched alkylene group having 1 to 12 carbon atoms are substituted with —O—, —S—, —NH—, —N(Q)—, or —CO—, where Q represents a substituent. Examples of the substituent include the above-described substituent E.

Suitable examples of the linear or branched alkylene group having 1 to 12 carbon atoms, represented by one aspect of SP¹ and SP², include a methylene group, an ethylene group, a propylene group, a butylene group, a pentylene group, a hexylene group, a methylhexylene group, and a heptylene group.

In Formula (I-1), Mes represents a group represented by Formula (I-2). Furthermore, in Formula (I-2), * represents a bonding position to Ar¹ or Ar².

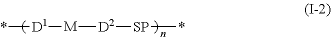

In Formula (I-2), M represents a 1,4-phenylene group, a 1,4-cyclohexylene group, a pyridine-2,5-diyl group, a pyrimidine-2,5-diyl group, a naphthalene-2,6-diyl group, a naphthalene-1,4-diyl group, a tetrahydronaphthalene-2,6-diyl group, a decahydronaphthalene-2,6-diyl group, or a 1,3-dioxane-2,5-diyl group. It should be noted that the hydrogen atom included in these groups may be substituted with a fluorine atom, a chlorine atom, a bromine atom, an iodine atom, a pentafluorosulfuranyl group, a cyano group, a nitro group, an isocyano group, a thioisocyano group, or an alkyl group having 1 to 20 carbon atoms.

In Formula (I-2), D¹ and D² each independently represent —O—, —S—, —OCH₂—, —CH₂CH₂—, —CO—, —COO—, —OCO—, —CO—S—, —OCO—O—, —CO—NH—, —NH—CO—, —SCH₂—, —CF₂O—, —CF₂S—, —CH═CH—COO—, —CH═CH—OCO—, —COO—CH₂CH₂—, —OCO—CH₂CH₂—, —COO—CH₂—, —OCO—CH₂—, —CH═CH—, —N═N—, —CH═N—, —CH═N—N═CH—, —CF═CF—, —C≡C—, or a single bond.

In Formula (I-2), SP represents a single bond, a linear or branched alkylene group having 1 to 12 carbon atoms, or a divalent linking group in which one or more of —CH₂—'s constituting the linear or branched alkylene group having 1 to 12 carbon atoms are substituted with —O—, —S—, —NH—, —N(Q)—, or —CO—, where Q represents a substituent. Examples of the substituent include the above-described substituent E.

Here, examples of the linear or branched alkylene group having 1 to 12 carbon atoms include the same ones described above for SP¹ and SP² in Formula (I-1).

In the present invention, SP is preferably the single bond.

In Formula (I-2), n represents an integer of 1 to 5, and is particularly preferably an integer of 1 to 3. It should be noted that in a case where n represents an integer of 2 to 5, a plurality of M's may be the same as or different from each other, a plurality of D¹'s may be the same as or different from each other, a plurality of D²'s may be the same as or different from each other, and a plurality of SP's may be the same as or different from each other.

In Formula (I-1), Ar¹ and Ar² each independently represent any ring structure selected from the group consisting of groups represented by Formulae (Ar-1) to (Ar-15). Furthermore, in Formulae (Ar-1) to (Ar-15), * represents a bonding position to Mes or SP¹ for Ar¹, and represents a bonding position to Mes or SP² for Ar².

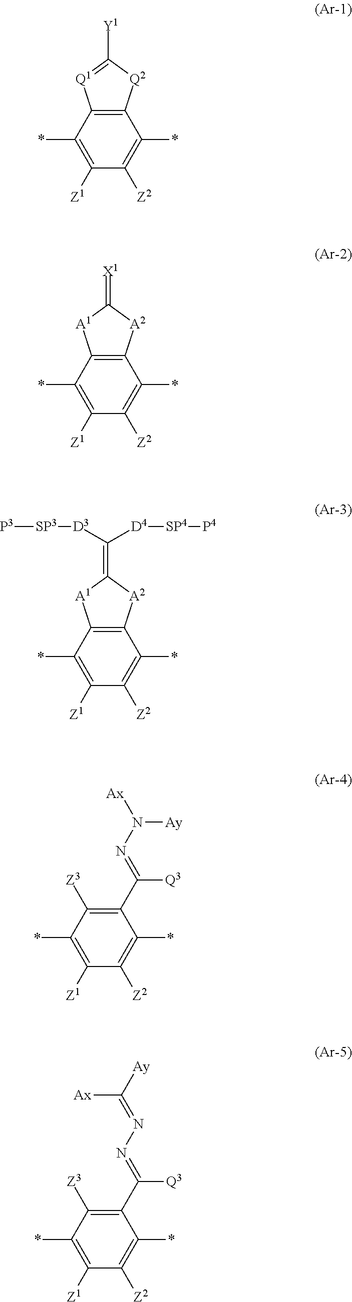

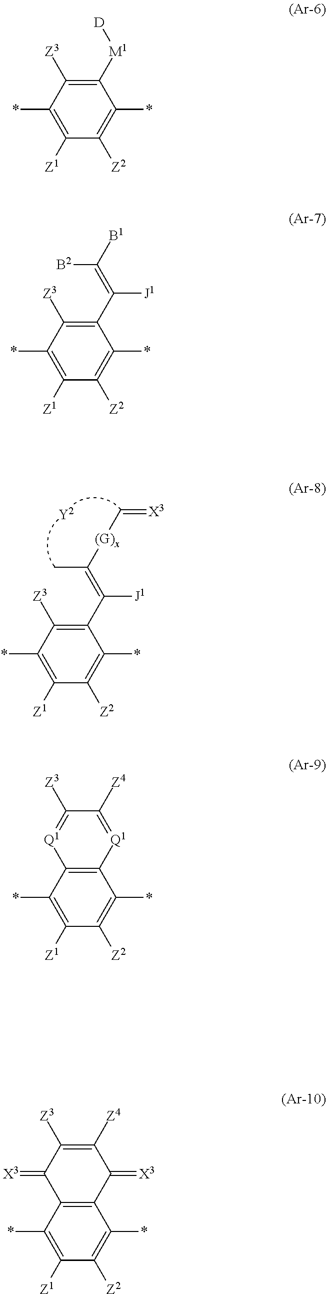

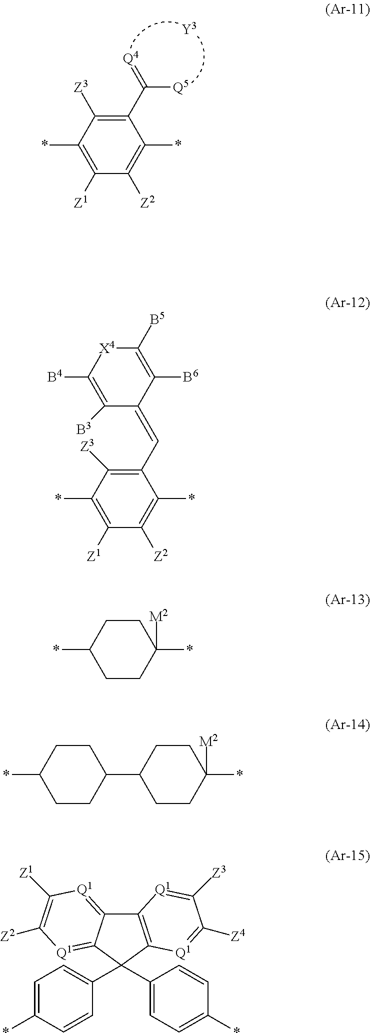

In Formulae (Ar-1), (Ar-9), and (Ar-15), Q¹ represents N or CH.

In Formula (Ar-1), Q² represents —S—, —O—, or —N(R⁶)—, where R⁶ represents a hydrogen atom or an alkyl group having 1 to 6 carbon atoms, and
specific examples of the alkyl group having 1 to 6 carbon atoms, represented by one aspect of R⁶, include a methyl group, an ethyl group, a propyl group, an isopropyl group, an n-butyl group, an isobutyl group, a sec-butyl group, a tert-butyl group, an n-pentyl group, and an n-hexyl group.

In Formula (Ar-1), Y¹ represents an alkynyl group having 2 to 16 carbon atoms, an aromatic hydrocarbon group having 6 to 12 carbon atoms, or an aromatic heterocyclic group having 3 to 12 carbon atoms, each of which may have a substituent.

Examples of the alkynyl group having 2 to 16 carbon atoms, represented by one aspect of Y¹, include an ethynyl group, a propargyl group, and a trimethylsilylethynyl group.

Examples of the aromatic hydrocarbon group having 6 to 12 carbon atoms, represented by one aspect of Y¹, include aryl groups such as a phenyl group, a 2,6-diethylphenyl group, and a naphthyl group.

Examples of the aromatic heterocyclic group having 3 to 12 carbon atoms, represented by one aspect of Y¹, include heteroaryl groups such as a thienyl group, a thiazolyl group, a furyl group, and a pyridyl group.

In addition, examples of the substituent which may be contained in Y¹ include the above-described substituent E.

In addition, in Formulae (Ar-1) to (Ar-12) and (Ar-15), Z¹, Z², Z³, and Z⁴ each independently represent a hydrogen atom, a monovalent aliphatic hydrocarbon group having 1 to 20 carbon atoms, a monovalent alicyclic hydrocarbon group having 3 to 20 carbon atoms, a monovalent aromatic group having 6 to 18 π electrons, a halogen atom, a cyano group, a nitro group, —OR⁷, —NR⁸R⁹, —SR¹⁰, —COOR¹¹, or —COR¹², where R⁷ to R¹² each independently represent a hydrogen atom or an alkyl group having 1 to 6 carbon atoms. It should be noted that Z¹ and Z², or Z³ and Z⁴ may be bonded to each other to form an aromatic ring.

As the monovalent aliphatic hydrocarbon group having 1 to 20 carbon atoms, an alkyl group having 1 to 15 carbon atoms is preferable, an alkyl group having 1 to 8 carbon atoms is more preferable, and specifically a methyl group, an ethyl group, an isopropyl group, a tert-pentyl group (1,1-dimethylpropyl group), a tert-butyl group, or a 1,1-dimethyl-3,3-dimethyl-butyl group is still more preferable, and the methyl group, the ethyl group, or the tert-butyl group is particularly preferable.

Examples of the monovalent alicyclic hydrocarbon group having 3 to 20 carbon atoms include monocyclic saturated hydrocarbon groups such as a cyclopropyl group, a cyclobutyl group, a cyclopentyl group, a cyclohexyl group, a cycloheptyl group, a cyclooctyl group, a cyclodecyl group, a methylcyclohexyl group, and an ethylcyclohexyl group; monocyclic unsaturated hydrocarbon groups such as a cyclobutenyl group, a cyclopentenyl group, a cyclohexenyl group, a cycloheptenyl group, a cyclooctenyl group, a cyclodecenyl group, a cyclopentadienyl group, a cyclohexadienyl group, a cyclooctadienyl group, and a cyclodecadiene; and polycyclic saturated hydrocarbon groups such as a bicyclo [2.2.1]heptyl group, a bicyclo [2.2.2]octyl group, a tricyclo [5.2.1.0^{2,6}] decyl group, a tricyclo [3.3.1.1^{3,7}] decyl group, a tetracyclo [6.2.1.1^{3,6}.0^{2,7}] dodecyl group, and an adamantyl group.

Specific examples of the monovalent aromatic group having 6 to 18 π electrons include a phenyl group, a 2,6-diethylphenyl group, a naphthyl group, a biphenyl group, and a thiophene ring group, and an aryl group having 6 to 12 carbon atoms (in particular, a phenyl group) or the thiophene ring group is preferable.

Examples of the halogen atom include a fluorine atom, a chlorine atom, a bromine atom, and an iodine atom, and among these, the fluorine atom, the chlorine atom, or the bromine atom is preferable.

On the other hand, specific examples of the alkyl group having 1 to 6 carbon atoms, represented by each of R⁷ to R¹², include a methyl group, an ethyl group, a propyl group, an isopropyl group, an n-butyl group, an isobutyl group, a sec-butyl group, a tert-butyl group, an n-pentyl group, and an n-hexyl group.

As described above, Z¹ and Z² may be bonded to each other to form an aromatic ring, and examples of the structure in a case where Z¹ and Z² in Formula (Ar-1) are bonded to each other to form an aromatic ring include a group represented by Formula (Ar-1a). Furthermore, in Formula (Ar-1a), examples of Q¹, Q², and Y¹ include the same ones as those described in Formula (Ar-1).

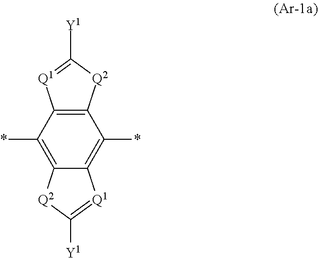

In Formulae (Ar-2) and (Ar-3), A¹ and A² each independently represent a group selected from the group consisting of —CW¹W²—, —O—, —N(R¹³)—, —S—, and —CO—, where W¹ and W² each independently represent a hydrogen atom, a halogen atom, or an alkyl group having 1 to 6 carbon atoms, and R¹³ represents a hydrogen atom or a substituent.

Examples of the substituent represented by one aspect of R¹³ include the above-described substituent E.

In Formula (Ar-2), X¹ represents a non-metal atom of Groups 14 to 16. It should be noted that a hydrogen atom or a substituent may be bonded to the non-metal atom.

Examples of the non-metal atom of Groups 14 to 16 represented by X¹ include an oxygen atom, a sulfur atom, a nitrogen atom to which a hydrogen atom or a substituent is bonded [═N—R^{N1}, where R^{N1} represents a hydrogen atom or a substituent], and a carbon atom to which a hydrogen atom or a substituent is bonded [═C—(R^{C1})₂, where R^{C1} represents a hydrogen atom or a substituent].

Specific examples of the substituent include an alkyl group, an alkoxy group, an alkyl-substituted alkoxy group, a cyclic alkyl group, an aryl group (for example, a phenyl group and a naphthyl group), a cyano group, an amino group, a nitro group, an alkylcarbonyl group, a sulfo group, and a hydroxyl group.

In Formula (Ar-3), D³ and D⁴ each independently represent a single bond, or a divalent linking group consisting of —CO—, —O—, —S—, —C(═S)—, —CR¹R²—, —CR³═CR⁴—, —NR⁵—, or a combination of two or more of these groups, where R¹ to R⁵ each independently represent a hydrogen atom, a fluorine atom, or an alkyl group having 1 to 4 carbon atoms.

Examples of the divalent linking group represented by one aspect of D³ and D⁴ include —CO—, —O—, —CO—O—, —C(═S)O—, —CR¹R²—, —CR¹R²—CR¹R²—, —O—CR¹R²—, —CR¹R²—O—CR¹R²—, —CO—O—CR¹CR²—, —O—CO—CR¹R²—, —CR¹R²—O—CO—CR¹R²—, —CR¹R²—CO—O—CR¹R²—, —NR⁵—CR¹R²—, and —CO—NR⁵—.

R¹, R², and R⁵ each independently represent a hydrogen atom, a fluorine atom, or an alkyl group having 1 to 4 carbon atoms.

Among these, any of —CO—, —O—, and —CO—O— is preferable.

In Formula (Ar-3), SP³ and SP⁴ each independently represent a single bond, a linear or branched alkylene group having 1 to 12 carbon atoms, or a divalent linking group in which one or more of —CH₂—'s constituting the linear or branched alkylene group having 1 to 12 carbon atoms are substituted with —O—, —S—, —NH—, —N(Q)—, or —CO—, where Q represents a substituent. Examples of the substituent include the above-described substituent E.

Here, examples of the linear or branched alkylene group having 1 to 12 carbon atoms include the same ones described above for SP¹ and SP² in Formula (I-1).

In Formula (Ar-3), P³ and P⁴ each independently represent a monovalent organic group.

Examples of the monovalent organic group include the same ones (including the polymerizable groups) as described above for P¹ and P² in Formula (I-1).

In Formulae (Ar-4) and (Ar-5), Ax represents an organic group having 2 to 30 carbon atoms, which has at least one aromatic ring selected from the group consisting of an aromatic hydrocarbon ring and an aromatic heterocyclic ring.

In Formulae (Ar-4) and (Ar-5), Ay represents a hydrogen atom, an alkyl group having 1 to 12 carbon atoms, which may have a substituent, or an organic group having 2 to 30 carbon atoms, which has at least one aromatic ring selected from the group consisting of an aromatic hydrocarbon ring and an aromatic heterocyclic ring.

Here, the aromatic ring in each of Ax and Ay may have a substituent (for example, the above-described substituent E), and Ax and Ay may be bonded to each other to form a ring.

Examples of each of Ax and Ay include those described in paragraphs [0039] to [0095] of WO2014/010325A.

In Formulae (Ar-4) and (Ar-5), Q³ represents a hydrogen atom or an alkyl group having 1 to 12 carbon atoms which may have a substituent.

Furthermore, specific examples of the alkyl group having 1 to 12 carbon atoms, represented by one aspect of Q³, include a methyl group, an ethyl group, a propyl group, an isopropyl group, an n-butyl group, an isobutyl group, a sec-butyl group, a tert-butyl group, an n-pentyl group, and an n-hexyl group.

In addition, examples of the substituent which may be contained in the alkyl group having 1 to 12 carbon atoms include the above-described substituent E.

In Formula (Ar-6), M¹ represents —NR¹⁴—, —CH═CH—, —N═CH—, —CH═N—, —N═N—, or —C≡C—, where R¹⁴ represents a hydrogen atom or an alkyl group having 1 to 6 carbon atoms.

Among these, —CH═CH— or —C═C— is preferable.

In Formula (Ar-6), D represents a group represented by any of Formulae (D-1) to (D-10) which may have a substituent. Furthermore, in Formulae (D-1) to (D-10), * represents a bonding position to M1.

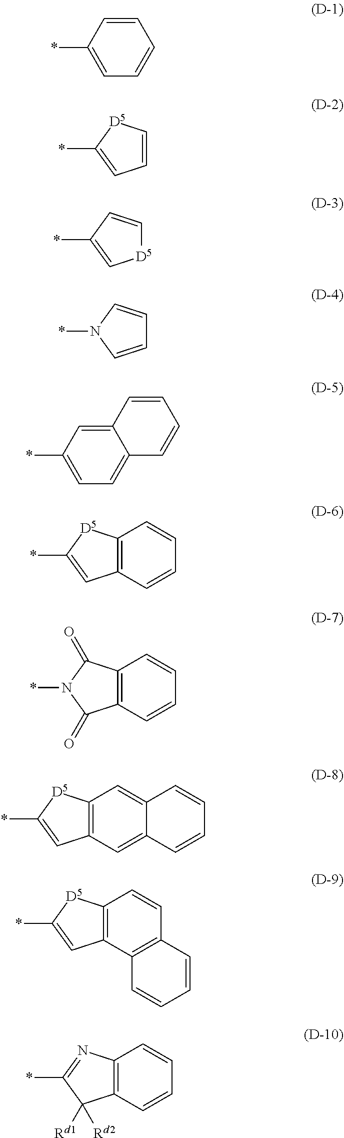

In Formulae (D-2), (D-3), (D-6), (D-8), and (D-9), D⁵ represents —O—, —S—, or —NR^{D1}—, where R^{D1} represents a hydrogen atom, an alkyl group having 1 to 5 carbon atoms, an alkanoyl group having 1 to 5 carbon atoms, or a phenyl group which may have a substituent (for example, the above-described substituent E).

In Formula (D-10), R^{d1} and R^{d2} each independently represent an alkyl group having 1 to 20 carbon atoms, which may have a substituent (for example, the above-described substituent E). It should be noted that one —CH₂— or two or more —CH₂—'s which are not adjacent to each other, constituting the alkyl group, may be each independently substituted with —O—, —S—, —CO—, —COO—, —OCO—, —CO—S—, —S—CO—, —SO₂—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C═C—.

In addition, R^{d1} and R^{d2} may be bonded to each other to form a 3- to 7-membered non-aromatic hydrocarbon ring, the non-aromatic hydrocarbon ring may have a substituent, and a carbon atom constituting the non-aromatic hydrocarbon ring may be substituted with a heteroatom.

As described above, the group represented by each of Formulae (D-1) to (D-10) may have a substituent, and specifically, the hydrogen atom bonded to a carbon atom constituting a ring structure in each of Formulae (D-1) to (D-10) may be substituted with a fluorine atom, a chlorine atom, a cyano group, a trifluoroacetyl group, a trifluoromethyl group, a phenyl group which may have a substituent, an alkyl group having 1 to 5 carbon atoms, an alkoxy group having 1 to 5 carbon atoms, an alkoxycarbonyl group having 1 to 5 carbon atoms, or an alkanoyl group having 1 to 5 carbon atoms.

In addition, —CH═ constituting a ring structure in each of Formulae (D-1) to (D-10) may be substituted with —N═.

In the present invention, from a reason that the reverse wavelength dispersibility of an optically anisotropic film thus formed is further improved, it is preferable that D in Formula (Ar-6) is a group represented by any of Formulae (D-1), (D-6), and (D-8) to (D-10), and from a reason that the aligning properties are improved, it is more preferable that D is the group represented by either of Formulae (D-1) and (D-6).

In Formulae (Ar-7) and (Ar-8), J¹ represents a hydrogen atom or an alkyl group.

J¹ is preferably the hydrogen atom or an alkyl group having 1 to 3 carbon atoms, more preferably the hydrogen atom, a methyl group, an ethyl group, a propyl group, or an isopropyl group, and still more preferably the hydrogen atom or the methyl group.

In Formula (Ar-7), B¹ represents —C(═X²)—B¹¹ or —CN. It should be noted that B¹¹ represents a substituent and X² represents ═O, ═S, ═NR¹⁵, or ═C(CN)₂, where R¹⁵ represents a substituent.

In addition, in Formula (Ar-7), B² represents a hydrogen atom or a substituent.

Examples of the substituent which is one aspect of B² in Formula (Ar-7) and the substituent represented by B¹¹ in “—C(═X²)—B¹¹” which is one aspect of B¹ in Formula (Ar-7) include the following substituents.

A substituted or unsubstituted, linear, branched, or cyclic alkyl group having 1 to 18 carbon atoms (preferably having 1 to 8 carbon atoms) (for example, methyl, ethyl, propyl, isopropyl, n-butyl, isobutyl, sec,-butyl, t-butyl, cyclohexyl, methoxyethyl, ethoxycarbonylethyl, cyanoethyl, diethylaminoethyl, hydroxyethyl, chloroethyl, acetoxyethyl, and trifluoromethyl);
an alkenyl group having 2 to 18 carbon atoms (preferably having 2 to 8 carbon atoms) (for example, vinyl);
an alkynyl group having 2 to 18 carbon atoms (preferably having 2 to 8 carbon atoms) (for example, ethynyl);
a substituted or unsubstituted aryl group having 6 to 18 carbon atoms (preferably having 6 to 10 carbon atoms) (for example, phenyl, 4-methylphenol, 4-methoxyphenyl, 4-carboxyphenyl, and 3,5-dicarboxyphenyl);
a substituted or unsubstituted aralkyl group having 7 to 18 carbon atoms (preferably having 7 to 12 carbon atoms) (for example, benzyl and carboxybenzyl);
a substituted or unsubstituted acyl group having 2 to 18 carbon atoms (preferably having 2 to 8 carbon atoms) (for example, acetyl, propionyl, butanoyl, and chloroacetyl);
a substituted or unsubstituted alkyl-or arylsulfonyl group having 1 to 18 carbon atoms (preferably having 1 to 8 carbon atoms) (for example, methanesulfonyl and p-toluenesulfonyl);
an alkylsulfinyl group having 1 to 18 carbon atoms (preferably having 1 to 8 carbon atoms) (for example, methanesulfinyl, ethanesulfinyl, and octanesulfinyl);
an alkoxycarbonyl group having 2 to 18 carbon atoms (preferably having 2 to 8 carbon atoms) (for example, methoxycarbonyl and ethoxycarbonyl);
an aryloxycarbonyl group having 7 to 18 carbon atoms (preferably having 7 to 12 carbon atoms) (for example, phenoxycarbonyl, 4-methylphenoxycarbonyl, and 4-methoxyphenylcarbonyl);
a substituted or unsubstituted alkoxy group having 1 to 18 carbon atoms (preferably having 1 to 8 carbon atoms) (for example, methoxy, ethoxy, n-butoxy, and methoxyethoxy);
a substituted or unsubstituted aryloxy group having 6 to 18 carbon atoms (preferably having 6 to 10 carbon atoms) (for example, phenoxy and 4-methoxyphenoxy);
an alkylthio group having 1 to 18 carbon atoms (preferably having 1 to 8 carbon atoms) (for example, methylthio and ethylthio);
an arylthio group having 6 to 10 carbon atoms (preferably having 1 to 8 carbon atoms) (for example, phenylthio);
a substituted or unsubstituted acyloxy group having 2 to 18 carbon atoms (preferably having 2 to 8 carbon atoms) (for example, acetoxy, ethylcarbonyloxy, cyclohexylcarbonyloxy, benzoyloxy, and chloroacetyloxy);
a substituted or unsubstituted sulfonyloxy group having 1 to 18 carbon atoms (preferably having 1 to 8 carbon atoms) (for example, methanesulfonyloxy);
a substituted or unsubstituted carbamoyloxy group having 2 to 18 carbon atoms (preferably having 2 to 8 carbon atoms) (for example, methylcarbamoyloxy and diethylcarbamoyloxy);
an unsubstituted amino group or a substituted amino group having 1 to 18 carbon atoms (preferably having 1 to 8 carbon atoms) (for example, methylamino, dimethylamino, diethylamino, anilino, methoxyphenylamino, chlorophenylamino, pyridylamino, methoxycarbonylamino, n-butoxycarbonylamino, phenoxycarbonylamino, methylcarbamoylamino, phenylcarbamoylamino, ethylthiocarbamoylamino, methylsulfamoylamino, phenylsulfamoylamino, acetylamino, ethylcarbonylamino, ethylthiocarbonylamino, cyclohexylcarbonylamino, benzoylamino, chloroacetylamino, methanesulfonylamino, and benzenesulfonylamino);
an amide group having 1 to 18 carbon atoms (preferably having 1 to 8 carbon atoms) (for example, acetamide, acetylmethylamide, and acetyloctylamide);
a substituted or unsubstituted ureido group having 1 to 18 carbon atoms (preferably having 1 to 8 carbon atoms) (for example, unsubstituted ureido, methylureido, ethylureido, and dimethylureido);
a substituted or unsubstituted carbamoyl group having 1 to 18 carbon atoms (preferably having 1 to 8 carbon atoms) (for example, unsubstituted carbamoyl, methylcarbamoyl, ethylcarbamoyl, n-butylcarbamoyl, t-butylcarbamoyl, dimethylcarbamoyl, morpholinocarbamoyl, and pyrrolidinocarbamoyl);
an unsubstituted sulfamoyl group or a substituted sulfamoyl group having 1 to 18 carbon atoms (preferably having 1 to 8 carbon atoms) (for example, methylsulfamoyl and phenylsulfamoyl);
a halogen atom (for example, fluorine, chlorine, and bromine); a hydroxyl group; a nitro group; a cyano group; a carboxyl group; and
a heterocyclic group (for example, an oxazole ring, a benzoxazole ring, a thiazole ring, a benzothiazole ring, an imidazole ring, a benzimidazole ring, an indolenine ring, a pyridine ring, a morpholine ring, a piperidine ring, a pyrrolidine ring, a sulfolane ring, a furan ring, a thiophene ring, a pyrazole ring, a pyrrole ring, a chromane ring, and a coumarin ring).

Among these, as the substituent represented by one aspect of B², a substituent having a Hammett's substituent constant (σp) value of 0.2 or more is preferable. The Hammett's substituent constants can be obtained by, for example, Chem. Rev. 91, 165 (1991). Particularly preferred substituents are a cyano group, a nitro group, an alkoxycarbonyl group, an acyl group, a carbamoyl group, a sulfamoyl group, an alkylsulfonyl group, and an arylsulfonyl group.

In addition, the substituent represented by B¹¹ is preferably the alkyl group, the aryl group, the alkoxy group, or the amino group.

As described above, X² in “—C(═X²)—B¹¹” which is one aspect of B¹ in Formula (Ar-7) represents ═O, ═S, ═NR¹⁵, or ═C(CN)₂, where R¹⁵ represents a substituent.

Here, examples of the substituent represented by R15 include those exemplified as the substituent represented by B¹¹ or B² described above. Among those, the substituent is preferably the aryl group, and more preferably phenyl.

In the present invention, X² is preferably ═O.

In Formula (Ar-8), Y² represents an atomic group necessary to form a carbon ring or a heterocyclic ring.

Y² is an atomic group composed of a carbon atom or a heteroatom (for example, a nitrogen atom, an oxygen atom, and a sulfur atom) that forms a heterocyclic ring in the main chain. That is, the main chain of this atomic group is a linking group consisting of two atoms selected from a carbon atom or heteroatoms (for example, a nitrogen atom, an oxygen atom, and a sulfur atom), and a hydrogen atom or a substituent (for example, the above-described substituent E) is bonded to the carbon atom or the heteroatom such that an appropriate valence is maintained. Furthermore, a double bond may be present in this atomic group.

In Formula (Ar-8), G represents an atomic group necessary to complete a conjugated double bond chain.

Examples of the conjugated double bond chain formed by G include —CH═CH—, —CH═CH—CH═CH—, and —CH═C(CH₃)—.

In Formula (Ar-8), x represents 0 or 1.

In Formulae (Ar-8) and (Ar-10), X³ represents ═O, ═S, ═NR¹⁵, or ═C(CN)₂, where R¹⁵ represents a substituent.

Examples of X³ include the same ones as X² in “—C(═X²)—B¹¹” which is one aspect of B¹ in Formula (Ar-7).

In addition, examples of the substituent represented by R¹⁵ include those exemplified as the substituent represented by B¹¹ and B² described above. Among those, the substituent is preferably the aryl group, and more preferably phenyl.

In Formula (Ar-8), the ring structure composed of Y², —C(═C)—(G)x—C(═X³)— [hereinafter abbreviated as “W¹” for the sake of convenience] is preferably a 4- to 7-membered carbon ring or heterocyclic ring, and more preferably a 5- or 6-membered carbon ring or heterocyclic ring. In particular, from a reason that the synthesis of the compound is facilitated and the reverse wavelength dispersibility of an optically anisotropic film thus formed is further improved, it is still more preferable that x represents 0, X³ represents O or S, and the ring structure composed of Y² and W¹ is a 5- or 6-membered carbon ring or heterocyclic ring.

These carbon rings or heterocyclic rings may have a substituent, and may further form a fused ring with another 4- to 7-membered ring.

Examples of the substituent include those exemplified as the substituent represented by B¹¹ and B² described above.

In addition, as the heteroatom constituting the heterocyclic ring, B, N, O, S, Se, and Te are preferable, and N, O, and S are more preferable.

Examples of the carbon ring composed of Y² and W¹ include the following rings. Furthermore, in the examples, Ra and Rb each independently represent a hydrogen atom or a substituent.

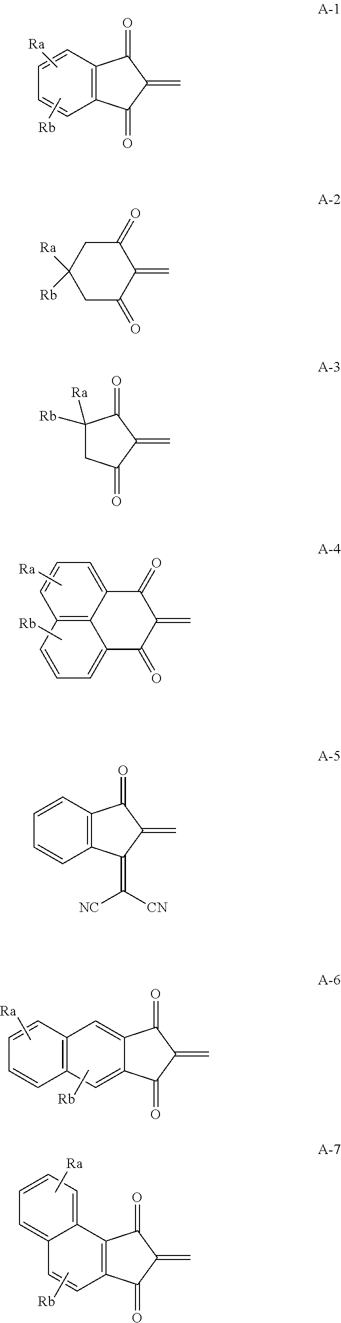

Among these carbon rings, the carbon rings represented by Formulae A-1, A-2, A-4, A-6, and A-7 are preferable, and the carbon rings represented by Formulae A-1 and A-2 are more preferable.

Examples of the heterocyclic ring composed of Y² and W¹ include the following rings. Furthermore, in the examples, Ra, Rb, and Rc each independently represent a hydrogen atom or a substituent.

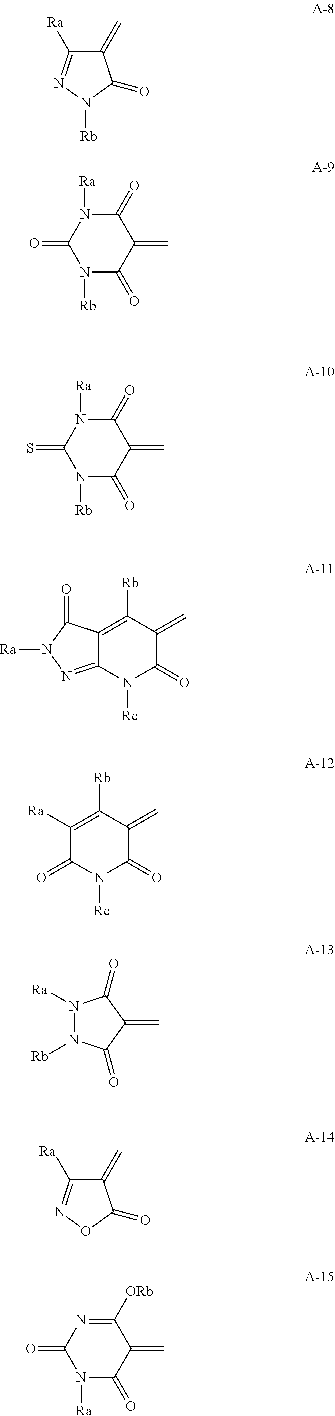

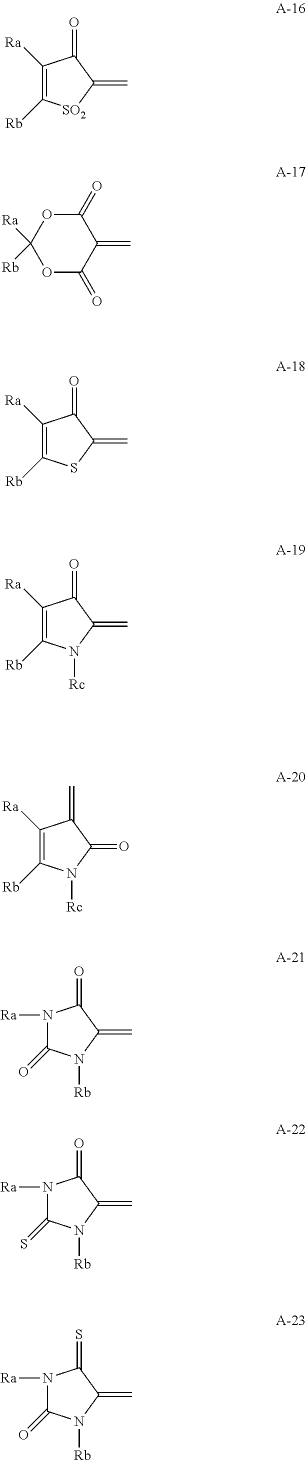

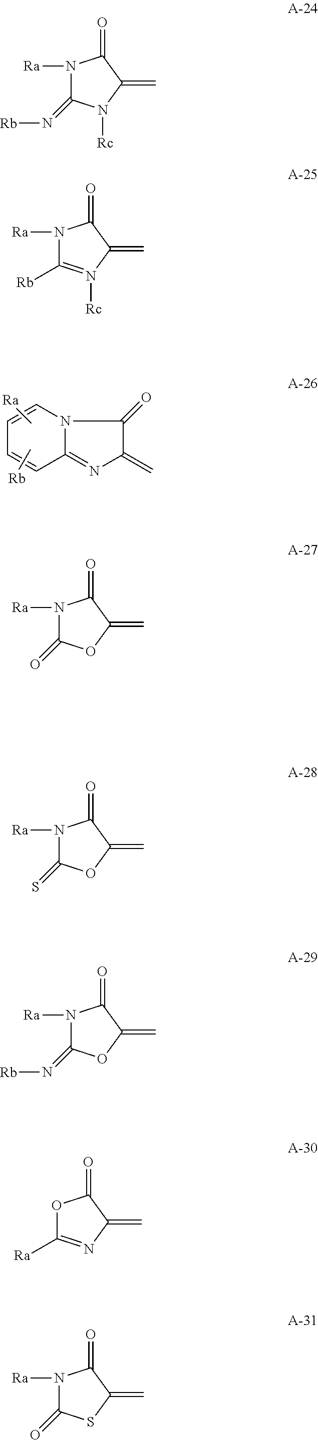

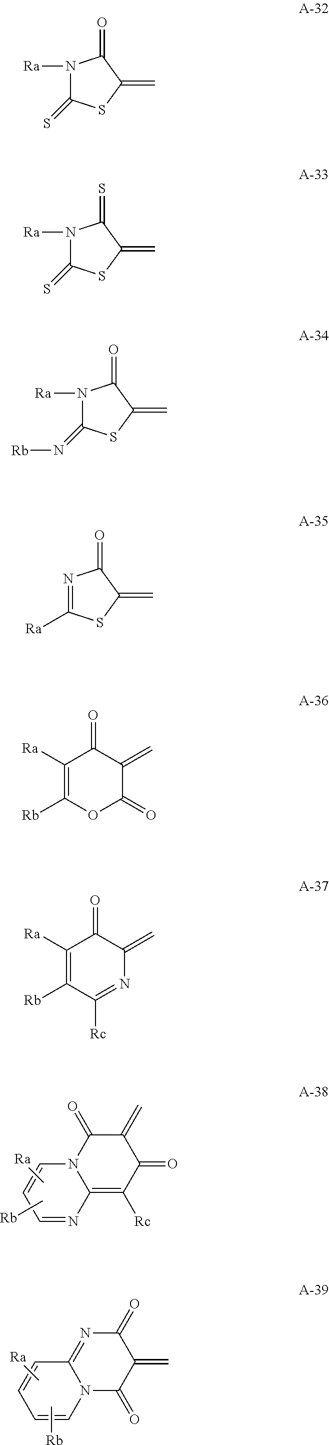

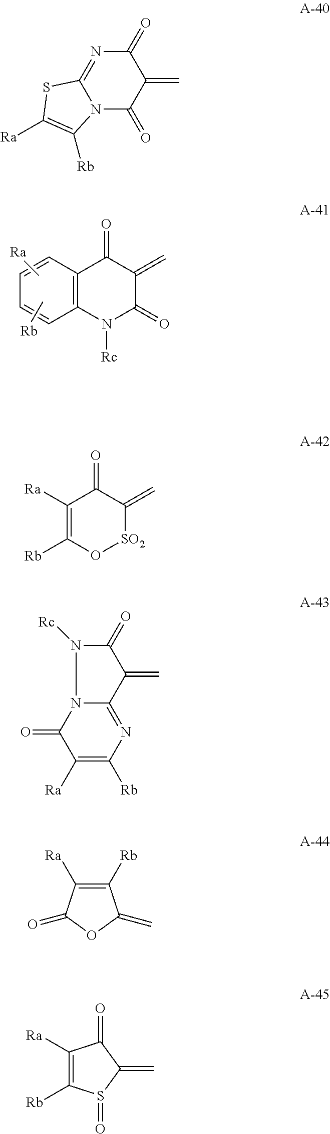

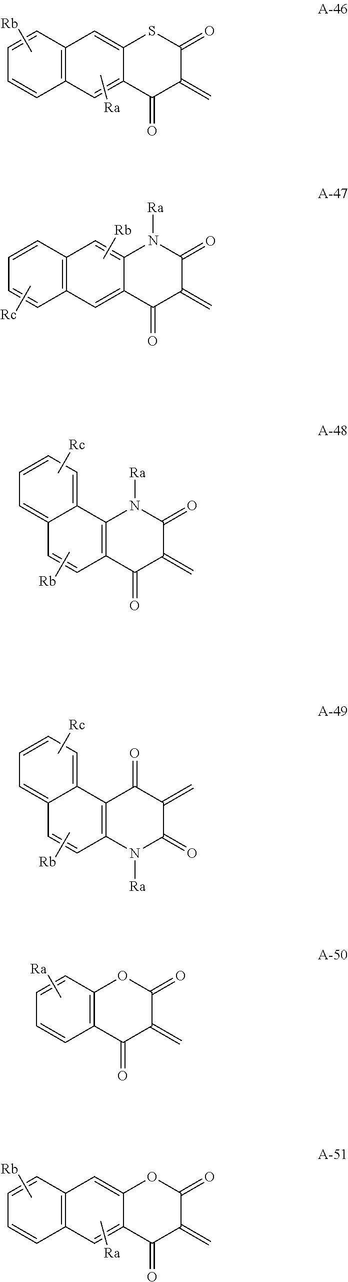

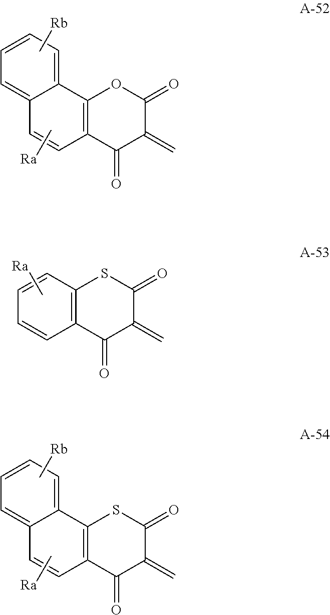

Among these heterocyclic rings, the heterocyclic rings represented by Formulae A-1, A-8, A-9, A-10, A-12, A-13, A-14, A-16, A-17, A-20, A-21, A-22, A-31, A-34, A-36, and A-45 are preferable, and the heterocyclic rings represented by Formulae A-1, A-8, A-13, A-14, A-16, A-17, A-20, A-21, A-22, A-31, and A-34 are more preferable.

Examples of the substituent represented by each of Ra, Rb, and Rc include those exemplified as the substituent represented by B¹¹ and B² described above. In particular, in a case of being substituted with a nitrogen atom, examples of the substituent include an alkyl group, a phenyl group, and a group represented by Formula (R-2). In a case of being substituted with a carbon atom, examples of the substituent include an alkyl group, an alkyl group in which one or more of —CH₂—'s constituting the alkyl group are substituted with —O—, —S—, —NH—, or —CO—, an alkenyl group, an alkynyl group, an aryl group, a cyano group, a nitro group, and the group represented by Formula (R-2).

-L¹⁰-R^{sp10}-Z¹⁰ Formula (R-2)

Here, in Formula (R-2),
L¹⁰ represents —O—, —S—, —OCH₂—, —CH₂O—, —CH₂CH₂—, —CO—, —COO—, —OCO—, —CO—S—, —S—CO—, —O—CO—O—, —CO—NH—, —NH—CO—, —OCO—NH—, —NH—COO—, —NH—CO—NH—, —NH—O—, —O—NH—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—,—CH₂—OCO—, —CH═CH—, —N═N—, —CH═N—, —N═CH—, —CH═N—N═CH—, —CF═CF—, —C≡C—, or single bond.

R^{sp10} represents an alkylene group having 1 to 20 carbon atoms or a single bond. It should be noted that one —CH₂— or two or more —CH₂—'s which are not adjacent to each other, constituting the alkylene group, may be each independently substituted with —O—, —COO—, —OCO—, —OCO—O—, —CO—NH—, —NH—CO—, —CH═CH—, or —C≡C—.

Z¹⁰ represents a polymerizable group.

In addition, Ra, Rb, and Rc may be linked to each other to form a carbon ring or a heterocyclic ring.

Examples of the carbon ring include saturated or unsaturated 4- to 7-membered carbon rings such as a cyclohexyl ring, a cyclopentyl ring, a cyclohexene ring, and a benzene ring.

In addition, examples of the heterocyclic ring include saturated or unsaturated 4- to 7-membered heterocyclic rings such as a piperidine ring, a piperazine ring, a morpholine ring, a tetrahydrofuran ring, a furan ring, a thiophene ring, a pyridine ring, and a pyrazine ring.

These carbon rings or heterocyclic rings may further have a substituent. Examples of the substituent include those exemplified as the substituent represented by B¹¹ and B² described above.

In Formula (Ar-11), Q⁴ represents ═N— or ═CR¹⁶—, where R¹⁶ represents a hydrogen atom or a substituent.

Examples of the substituent represented by one aspect of R¹⁶ include the above-described substituent E.

In addition, in Formula (Ar-11), Q⁵ represents a non-metal atom of Groups 14 to 16. It should be noted that a hydrogen atom or a substituent may be bonded to the non-metal atom.

Examples of Q⁵ include the same ones described above for X¹ in Formula (Ar-2).

In Formula (Ar-11), Y³ represents an atomic group necessary to form a carbon ring or a heterocyclic ring with Q⁴ and Q⁵.

The ring formed from Q⁴, Q⁵, and Y³ may be fused with another ring.

The ring formed from Q⁴, Q⁵, and Y³ is preferably a nitrogen-containing 5- or 6-membered ring fused with a benzene ring.

In Formula (Ar-12), B³, B⁴, B⁵, and B⁶ each independently represent a hydrogen atom or a substituent.

Examples of the substituent represented by one aspect of B³, B⁴, B⁵, and B⁶ include the above-described substituent E.

In Formula (Ar-12), X⁴ represents —O—, —S—, or —NR¹⁷—, where R¹⁷ represents a hydrogen atom or a substituent.

Examples of the substituent represented by one aspect of R¹⁷ include the above-described substituent E.

In Formulae (Ar-13) and (Ar-14), M² represents an alkynyl group.

Examples of the alkynyl group include an ethynyl group, a 1-propynyl group, a 2-propynyl group, a 2-butynyl group, a 3-butynyl group, a 3-pentynyl group, a 4-pentynyl group, a 1-hexynyl group, a 5-hexynyl group, and these group in which some or all of hydrogen atoms are substituted with substituents.

Specific suitable examples of the compound represented by Formula (I-1) include compounds represented by Formulae (1) to (160).

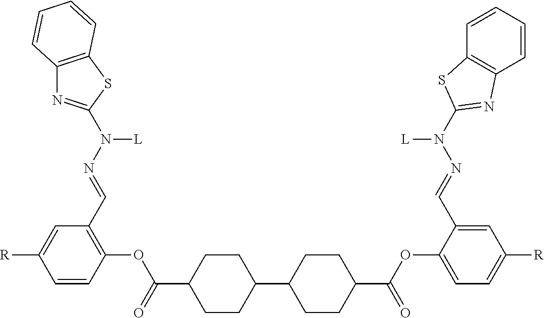

| No. | R | L |
|---|---|---|
| (1) | 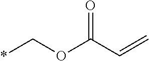 [SMILES: *COC(=O)C=C] | 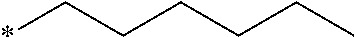 [SMILES: *CCCCCC] |
| (2) | 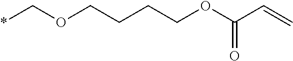 [SMILES: *COCCCCOC(=O)C=C] | 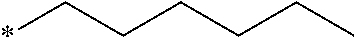 [SMILES: *CCCCCC] |
| (3) | 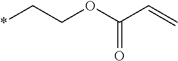 [SMILES: *CCOC(=O)C=C] | 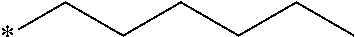 [SMILES: *CCCCCC] |
| (4) | 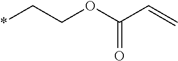 [SMILES: *CCOC(=O)C=C] | 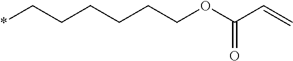 [SMILES: *CCCCCCOC(=O)C=C] |
| (5) | 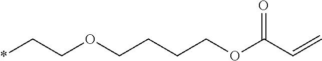 [SMILES: *CCOCCCCOC(=O)C=C] | 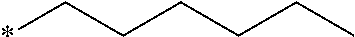 [SMILES: *CCCCCC] |
| (6) | 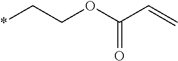 [SMILES: *CCOC(=O)C=C] | H |
| (7) | 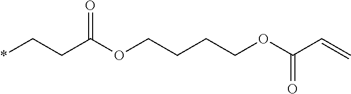 [SMILES: *CCC(=O)OCCCCOC(=O)C=C] | 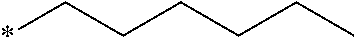 [SMILES: *CCCCCC] |
| (8) | 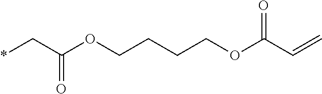 [SMILES: *CC(=O)OCCCCOC(=O)C=C] | 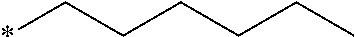 [SMILES: *CCCCCC] |
| (9) | 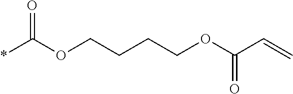 [SMILES: *C(=O)OCCCCOC(=O)C=C] | 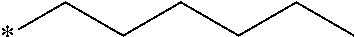 [SMILES: *CCCCCC] |
| (10) | 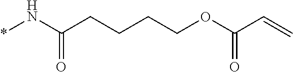 [SMILES: *NC(=O)CCCCOC(=O)C=C] | 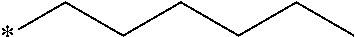 [SMILES: *CCCCCC] |
| (11) | 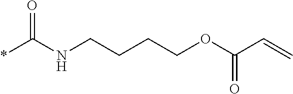 [SMILES: *C(=O)NCCCCOC(=O)C=C] | 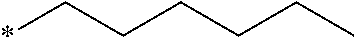 [SMILES: *CCCCCC] |

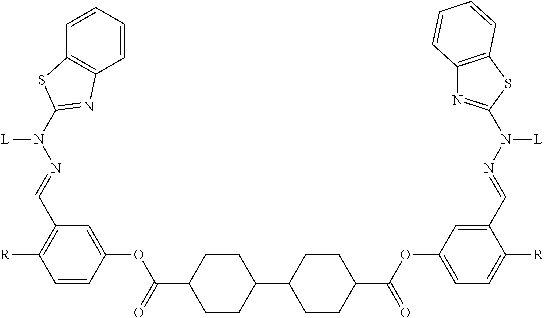

| No. | R | L |
|---|---|---|
| (12) | 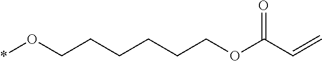 [SMILES: *OCCCCCCOC(=O)C=C] | 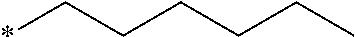 [SMILES: *CCCCCC] |

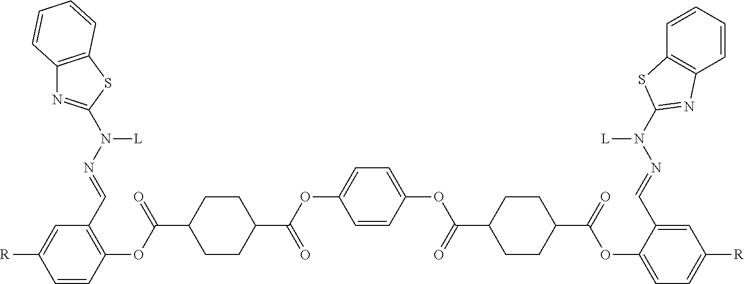

| No. | R | L |
|---|---|---|
| (13) | 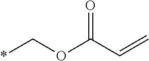 [SMILES: *COC(=O)C=C] | 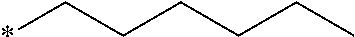 [SMILES: *CCCCCC] |
| (14) | 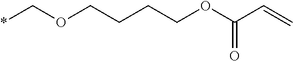 [SMILES: *COCCCCOC(=O)C=C] | 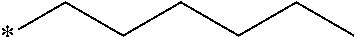 [SMILES: *CCCCCC] |
| (15) | 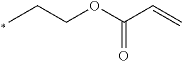 | 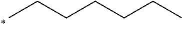 |
| (16) | 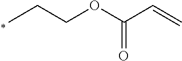 | 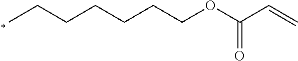 |
| (17) | 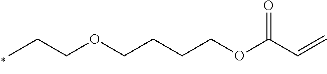 | 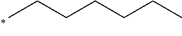 |
| (18) | 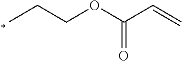 | H |
| (19) | 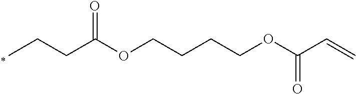 | 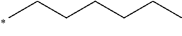 |
| (20) | 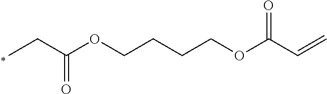 | 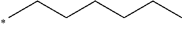 |
| (21) | 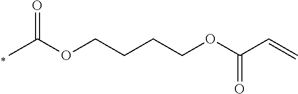 | 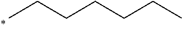 |
| (22) | 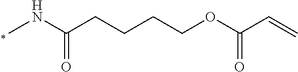 | 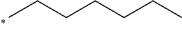 |
| (23) | 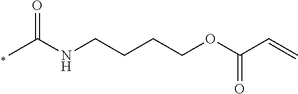 | 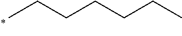 |

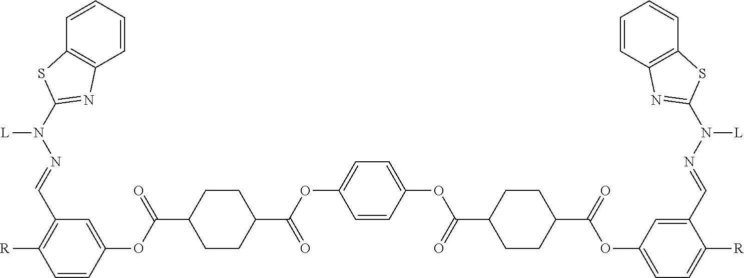

| No. | R | L |
|---|---|---|
| (24) | 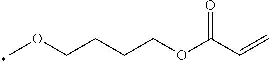 | 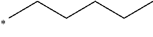 |

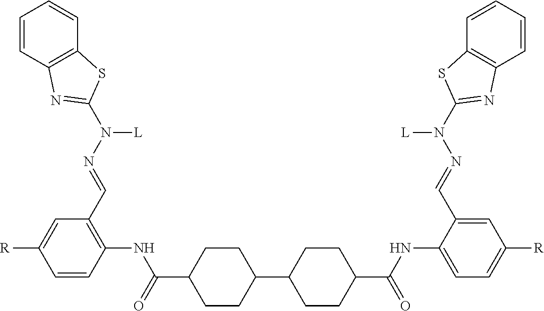

| No. | R | L |
|---|---|---|
| (25) | 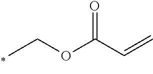 | 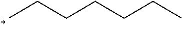 |
| (26) | 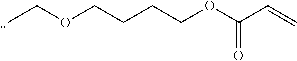 | 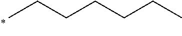 |
| (27) | 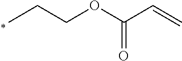 | 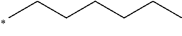 |
| (28) | 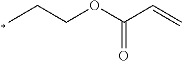 | 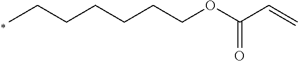 |
| (29) | 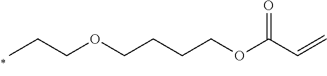 | 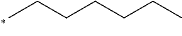 |
| (30) | 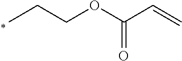 | H |
| (31) | 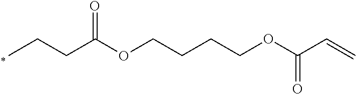 | 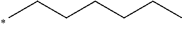 |
| (32) | 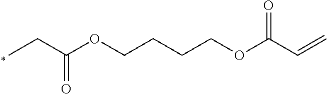 | 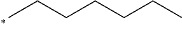 |
| (33) | 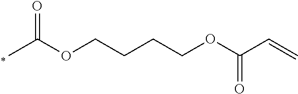 | 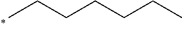 |
| (34) | 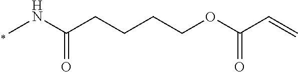 | 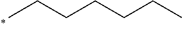 |
| (35) | 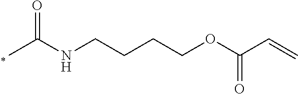 | 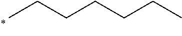 |
| (36) | 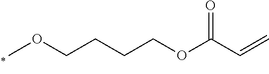 | 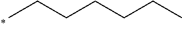 |
| (37) | 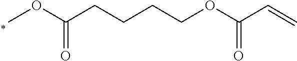 | 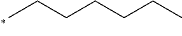 |

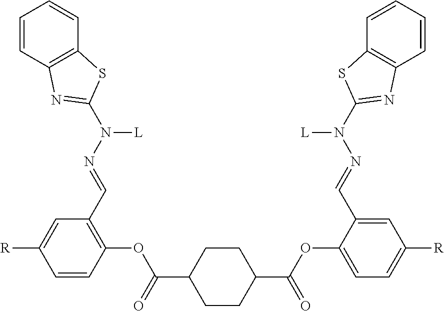

| No. | R | L |
|---|---|---|
| (38) | 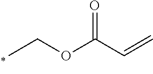 | 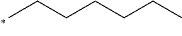 |
| (39) | 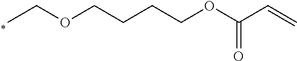 | 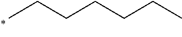 |
| (40) | 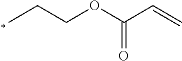 | 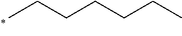 |
| (41) | 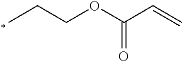 | 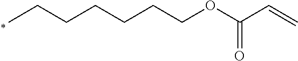 |
| (42) | 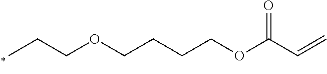 | 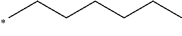 |
| (43) | 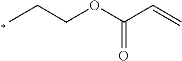 | H |
| (44) | 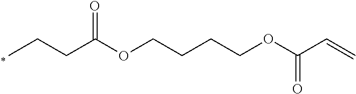 | 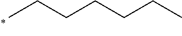 |
| (45) | 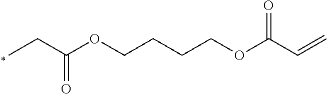 | 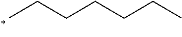 |
| (46) | 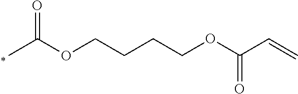 | 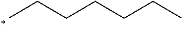 |
| (47) | 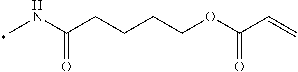 | 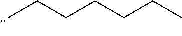 |
| (48) | 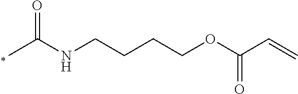 | 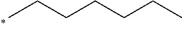 |
| (49) | 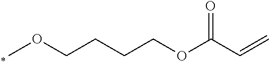 | 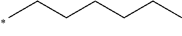 |
| (50) | 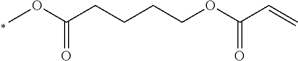 | 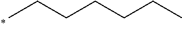 |

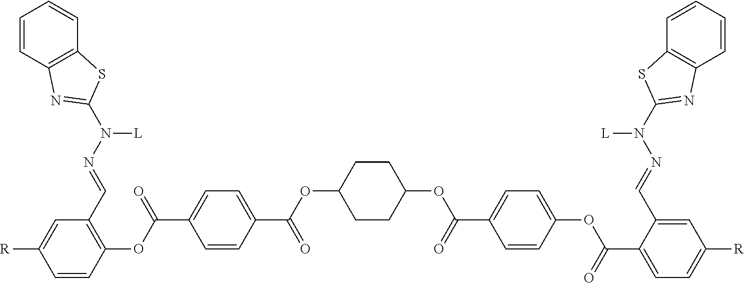

| No. | R | L |
|---|---|---|
| (51) | 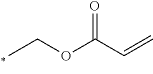 | 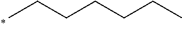 |
| (52) | 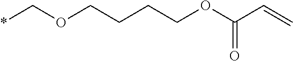 [SMILES: *COCCCCOC(=O)C=C] | 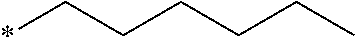 [SMILES: *CCCCCC] |
| (53) | 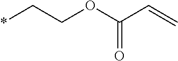 [SMILES: *CCOC(=O)C=C] | 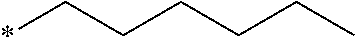 [SMILES: *CCCCCC] |
| (54) | 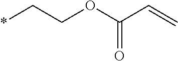 [SMILES: *CCOC(=O)C=C] | 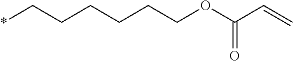 [SMILES: *CCCCCCOC(=O)C=C] |
| (55) | 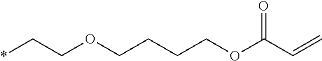 [SMILES: *CCOCCCCOC(=O)C=C] | 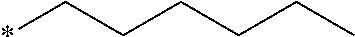 [SMILES: *CCCCCC] |
| (56) | 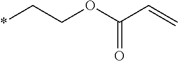 [SMILES: *CCOC(=O)C=C] | H |
| (57) | 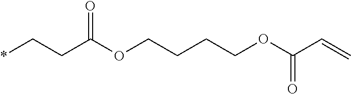 [SMILES: *CCC(=O)OCCCCOC(=O)C=C] | 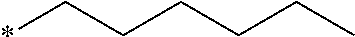 [SMILES: *CCCCCC] |
| (58) | 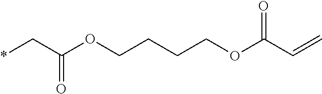 [SMILES: *CC(=O)OCCCCOC(=O)C=C] | 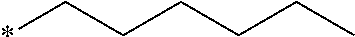 [SMILES: *CCCCCC] |
| (59) | 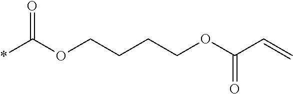 [SMILES: *C(=O)OCCCCOC(=O)C=C] | 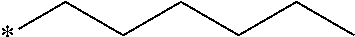 [SMILES: *CCCCCC] |
| (60) | 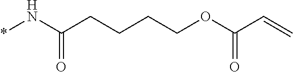 [SMILES: *NC(=O)CCCCOC(=O)C=C] | 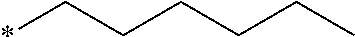 [SMILES: *CCCCCC] |
| (61) | 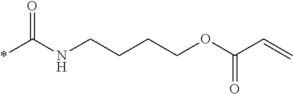 [SMILES: *C(=O)NCCCCOC(=O)C=C] | 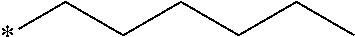 [SMILES: *CCCCCC] |

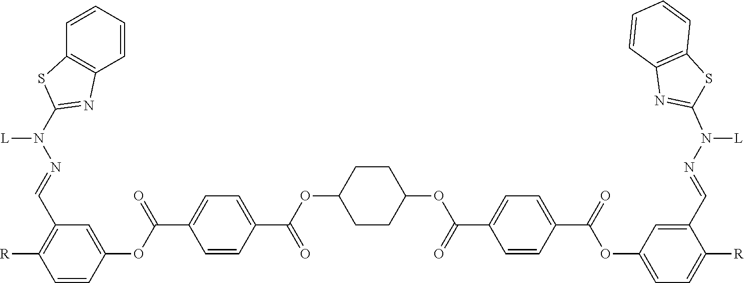

| No. | R | L |
|---|---|---|
| (62) | 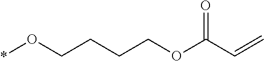 [SMILES: *OCCCCOC(=O)C=C] | 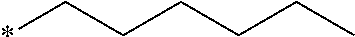 [SMILES: *CCCCCC] |
| (63) | 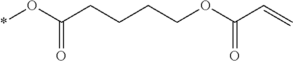 [SMILES: *OC(=O)CCCCOC(=O)C=C] | 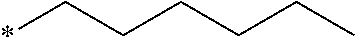 [SMILES: *CCCCCC] |

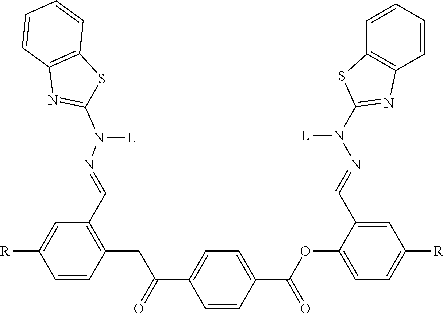

| No. | R | L |
|---|---|---|
| (64) | 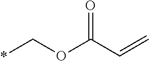 [SMILES: *COC(=O)C=C] | 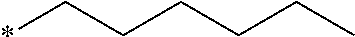 [SMILES: *CCCCCC] |
| (65) | 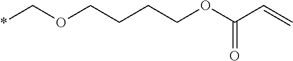 [SMILES: *COCCCCOC(=O)C=C] | 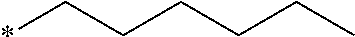 [SMILES: *CCCCCC] |
| (66) | 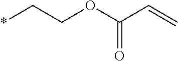 [SMILES: *CCOC(=O)C=C] | 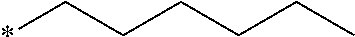 [SMILES: *CCCCCC] |
| (67) | 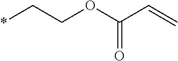 [SMILES: *CCOC(=O)C=C] | 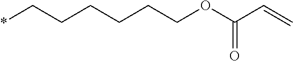 [SMILES: *CCCCCCOC(=O)C=C] |
| (68) | 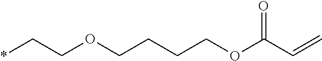 [SMILES: *CCOCCCCOC(=O)C=C] | 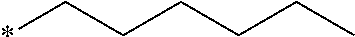 [SMILES: *CCCCCC] |
| (69) | 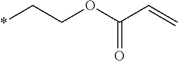 [SMILES: *CCOC(=O)C=C] | H |
| (70) | 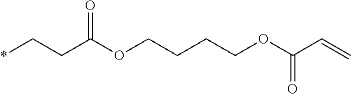 [SMILES: *CCC(=O)OCCCCOC(=O)C=C] | 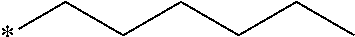 [SMILES: *CCCCCC] |
| (71) | 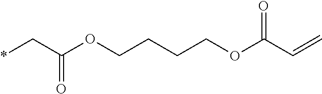 [SMILES: *CC(=O)OCCCCOC(=O)C=C] | 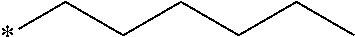 [SMILES: *CCCCCC] |
| (72) | 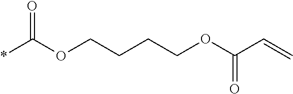 [SMILES: *C(=O)OCCCCOC(=O)C=C] | 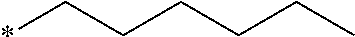 [SMILES: *CCCCCC] |
| (73) | 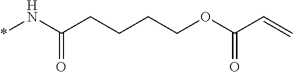 [SMILES: *NC(=O)CCCCOC(=O)C=C] | 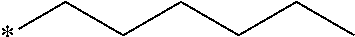 [SMILES: *CCCCCC] |
| (74) | 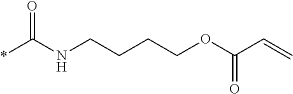 [SMILES: *C(=O)NCCCCOC(=O)C=C] | 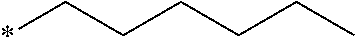 [SMILES: *CCCCCC] |

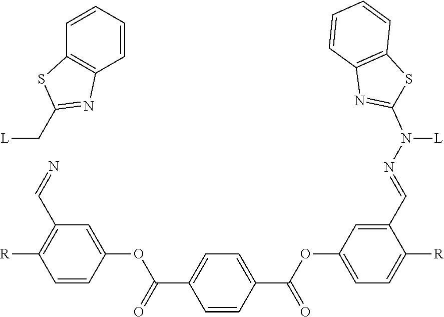

| No. | R | L |
|---|---|---|
| (75) | 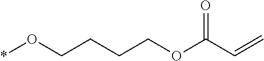 [SMILES: *OCCCCOC(=O)C=C] | 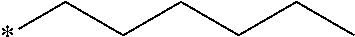 [SMILES: *CCCCCC] |
| (76) | 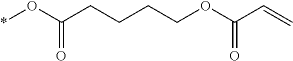 [SMILES: *OC(=O)CCCCOC(=O)C=C] | 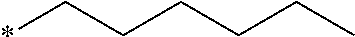 [SMILES: *CCCCCC] |

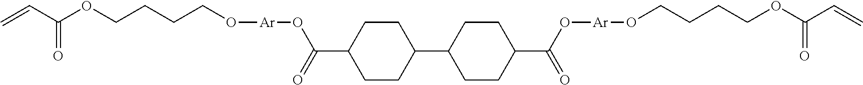

| No. | Ar |
|---|---|
| (77) | 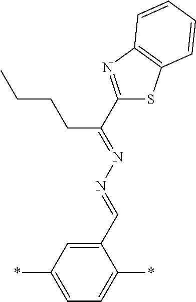 [SMILES: *c1ccc(*)c(/C=N/N=C(\CCCC)c2nc3ccccc3s2)c1] |
| (78) | 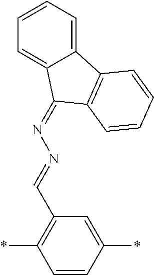 [SMILES: *c1ccc(*)c(/C=N/N=C2c3ccccc3-c3ccccc32)c1] |
| (79) | 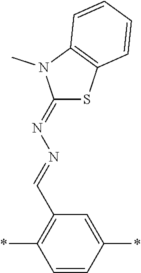 [SMILES: *c1ccc(*)c(/C=N/N=c2\sc3ccccc3n2C)c1] |

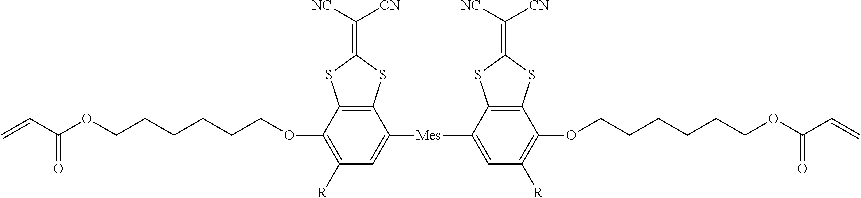

| No. | Mes | R |
|---|---|---|
| (80) | 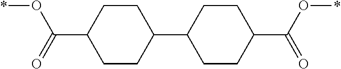 [SMILES: *OC(=O)C1CCC(C2CCC(C(=O)O*)CC2)CC1] | H |
| (81) | 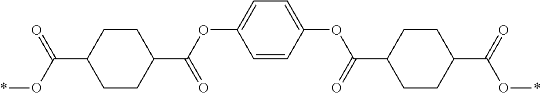 [SMILES: *OC(=O)C1CCC(C(=O)Oc2ccc(OC(=O)C3CCC(C(=O)O*)CC3)cc2)CC1] | H |
| (82) | 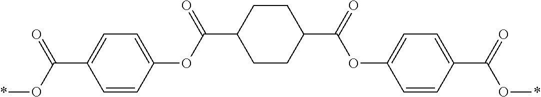 [SMILES: *OC(=O)c1ccc(OC(=O)C2CCC(C(=O)Oc3ccc(C(=O)O*)cc3)CC2)cc1] | H |
| (83) | 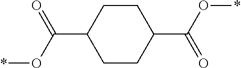 [SMILES: *OC(=O)C1CCC(C(=O)O*)CC1] | H |
| (84) | 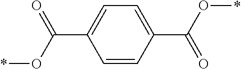 [SMILES: *OC(=O)c1ccc(C(=O)O*)cc1] | H |
| (85) | 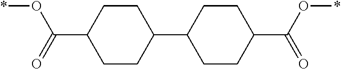 [SMILES: *OC(=O)C1CCC(C2CCC(C(=O)O*)CC2)CC1] | 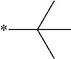 [SMILES: *C(C)(C)C] |
| (86) | 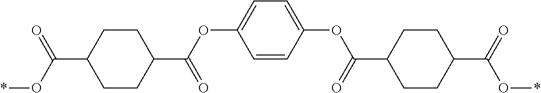 [SMILES: *OC(=O)C1CCC(C(=O)Oc2ccc(OC(=O)C3CCC(C(=O)O*)CC3)cc2)CC1] | 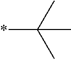 [SMILES: *C(C)(C)C] |
| (87) | 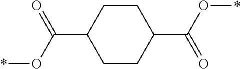 [SMILES: *OC(=O)C1CCC(C(=O)O*)CC1] | 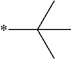 [SMILES: *C(C)(C)C] |

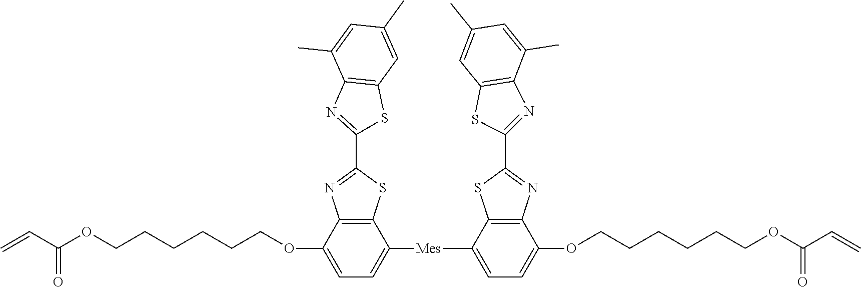

| No. | Mes |
|---|---|
| (88) | 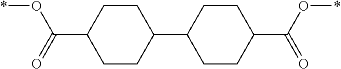 [SMILES: *OC(=O)C1CCC(C2CCC(C(=O)O*)CC2)CC1] |
| (89) | 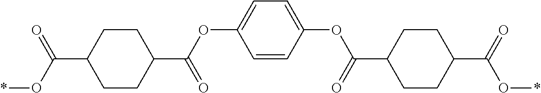 [SMILES: *OC(=O)C1CCC(C(=O)Oc2ccc(OC(=O)C3CCC(C(=O)O*)CC3)cc2)CC1] |
| (90) | 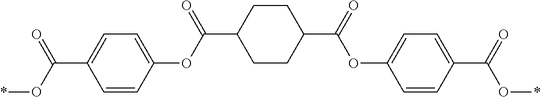 [SMILES: *OC(=O)c1ccc(OC(=O)C2CCC(C(=O)Oc3ccc(C(=O)O*)cc3)CC2)cc1] |
| (91) | 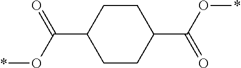 [SMILES: *OC(=O)C1CCC(C(=O)O*)CC1] |
| (92) | 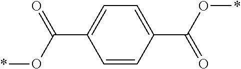 [SMILES: *OC(=O)c1ccc(C(=O)O*)cc1] |

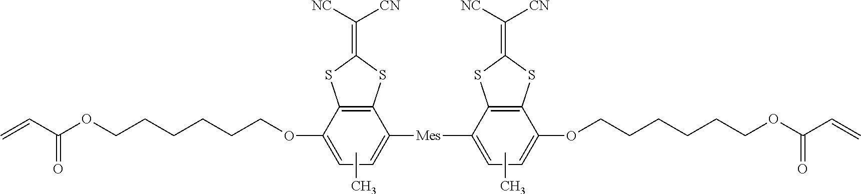

| No. | Mes |
|---|---|
| (93) | 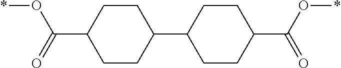 [SMILES: *OC(=O)C1CCC(C2CCC(C(=O)O*)CC2)CC1] |
| (94) | 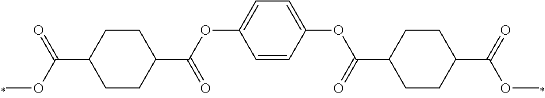 |
| (95) | 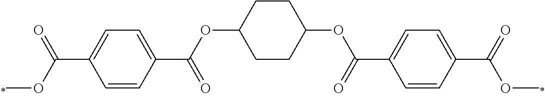 |
| (96) | 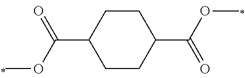 |
| (97) | 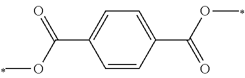 |

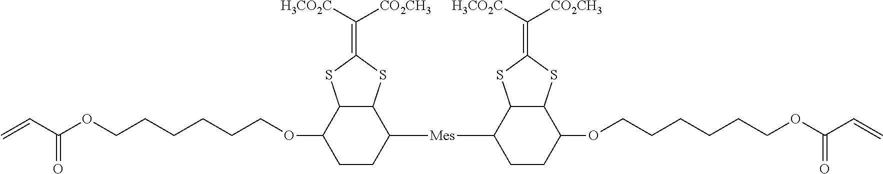

| No. | Mes |
|---|---|
| (98) | 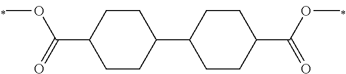 |
| (99) | 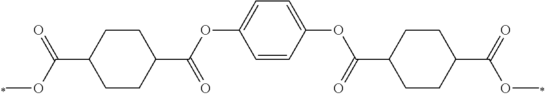 |
| (100) | 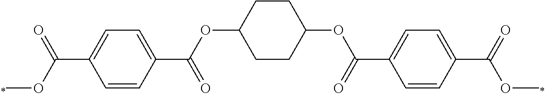 |
| (101) | 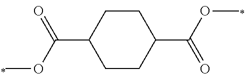 |
| (102) | 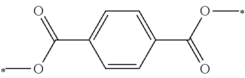 |

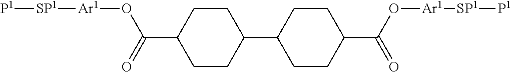

| No. | Ar¹—SP¹—P¹ |
|---|---|
| (103) | 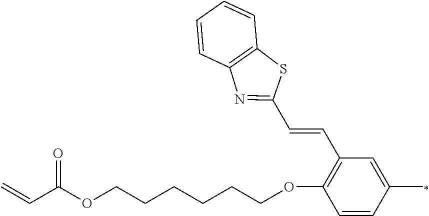 |
| (104) | 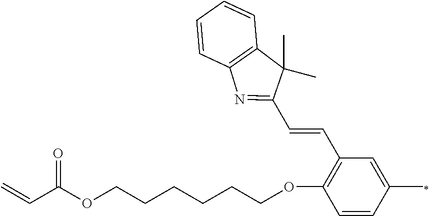 |
| (105) | 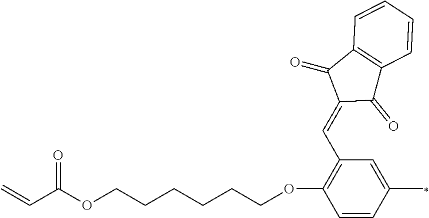 |
| (106) | 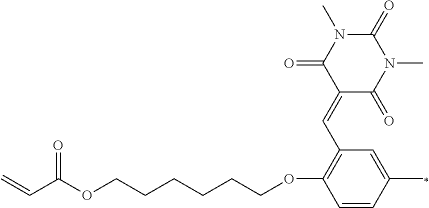 |
| (107) | 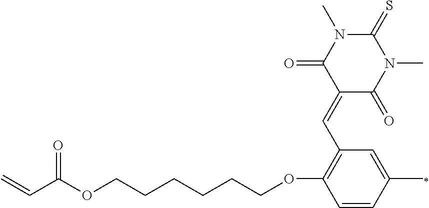 |
| (108) | 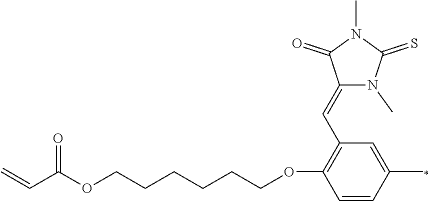 |
| (109) | 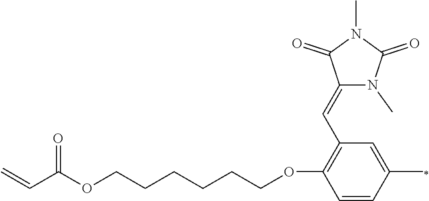 |
| (110) | 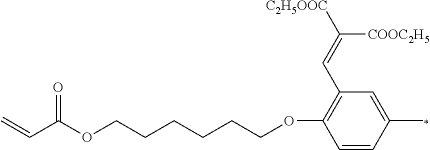 |
| (111) | 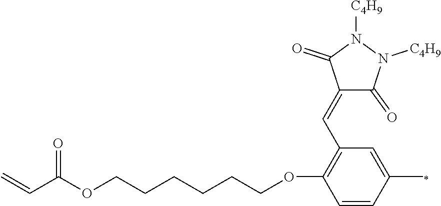 |
| (112) | 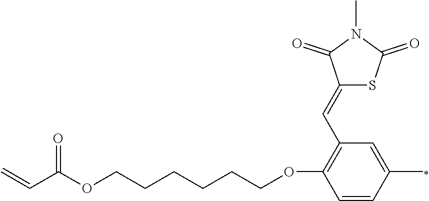 |
| (113) | 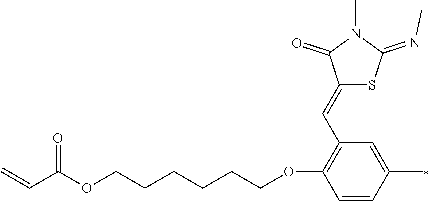 |
| (114) | 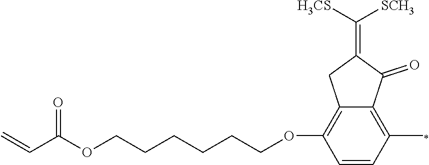 |
| (115) | 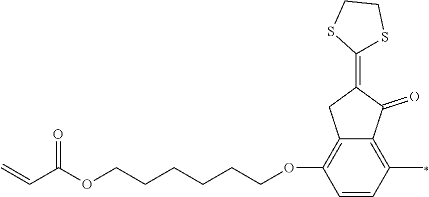 |
| (116) | 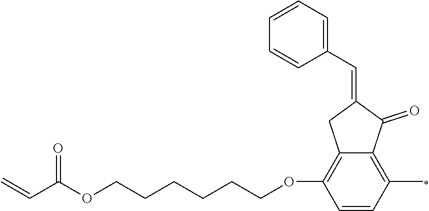 |
| (117) | 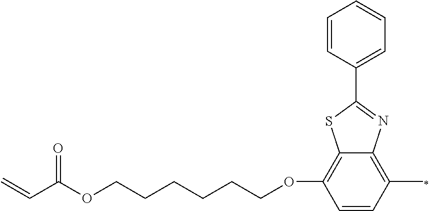 |
| (118) | 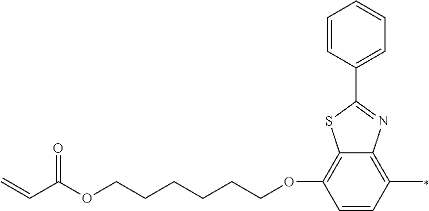 |
| (119) | 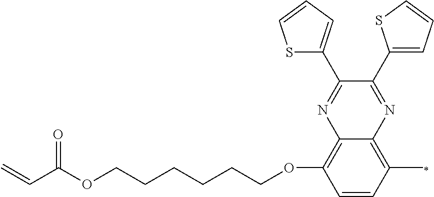 |
| (120) | 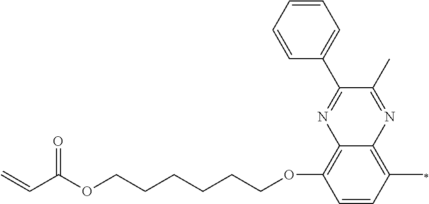 |
| (121) | 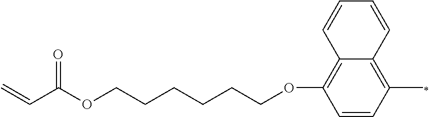 |
| (122) | 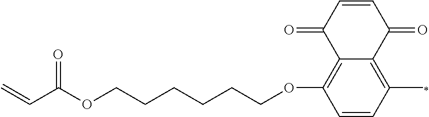 |
| (123) | 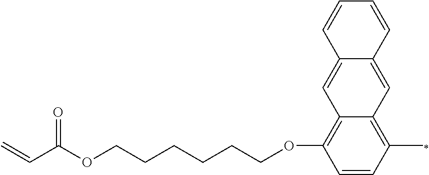 |
| (124) | 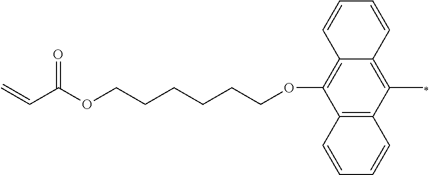 |
| (125) | 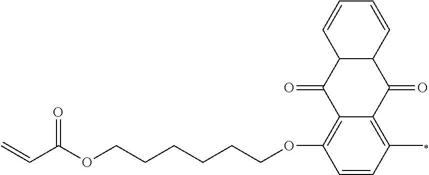 |
| (126) | 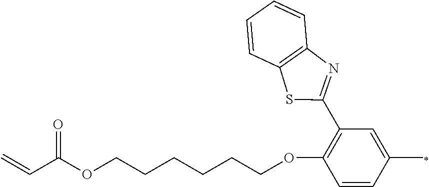 |
| (127) | 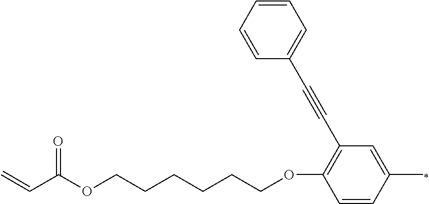 |
| (128) | 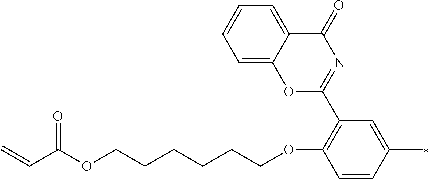 |
| (129) | 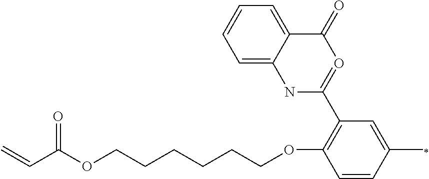 |
| (130) | 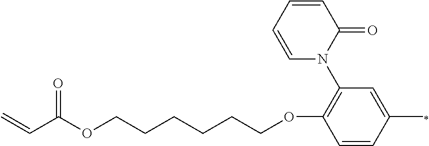 |
| (131) | 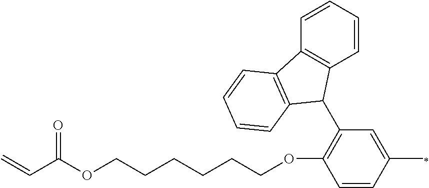 |
| (132) | 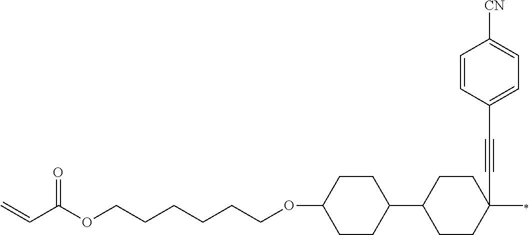 |
| (133) | 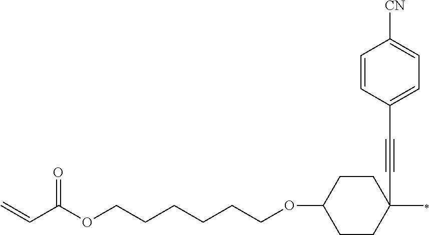 |
| (134) | 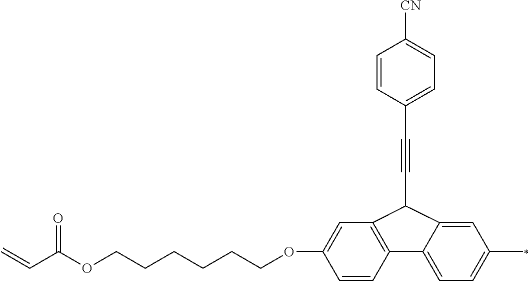 |
| (135) | 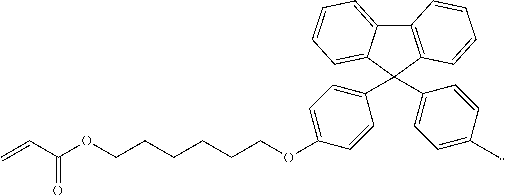 |
| (136) | 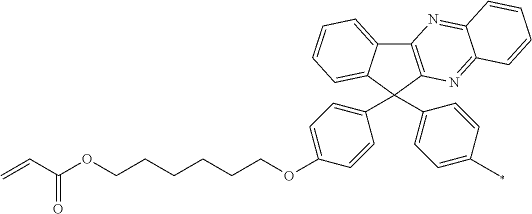 |
| (137) | 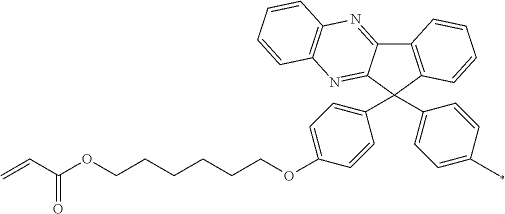 |
| (138) | 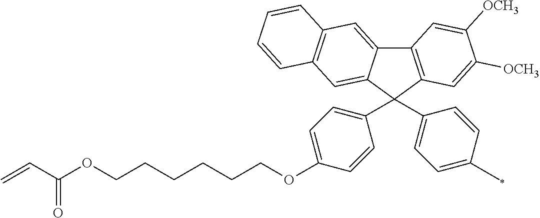 |
| (139) | 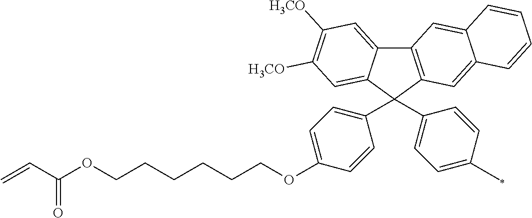 |
| (140) | 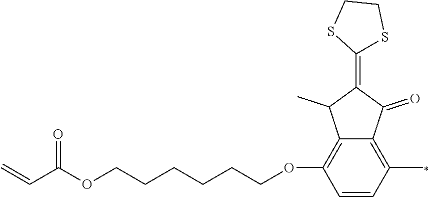 |
| (141) | 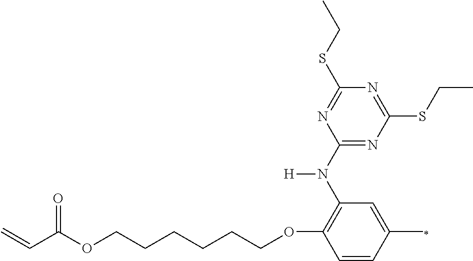 |

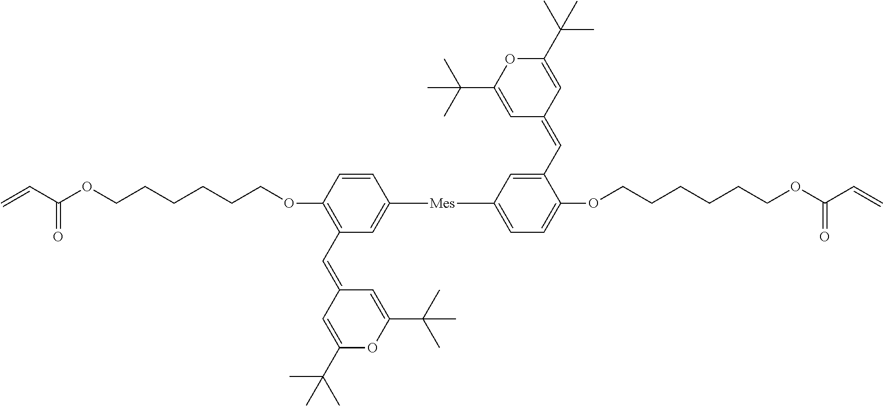

| No. | Mes |
|---|---|
| (142) | 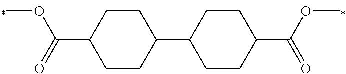 |
| (143) | 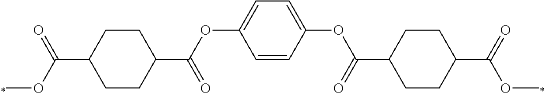 |
| (144) | 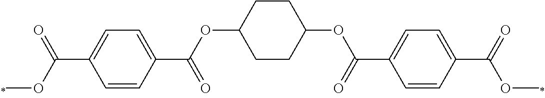 |
| (145) | 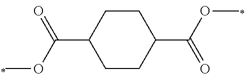 |
| (146) | 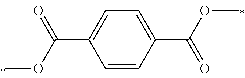 |

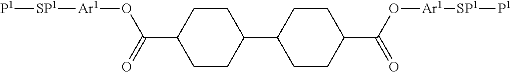

| No. | Ar¹—SP¹—P¹ |
|---|---|
| (147) | 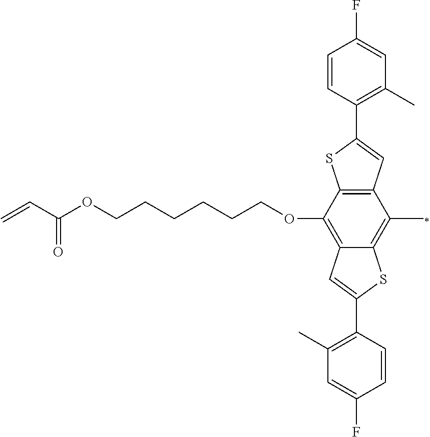 |
| (148) | 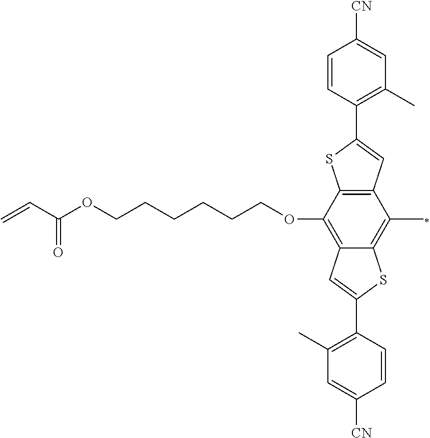 |
| (149) | 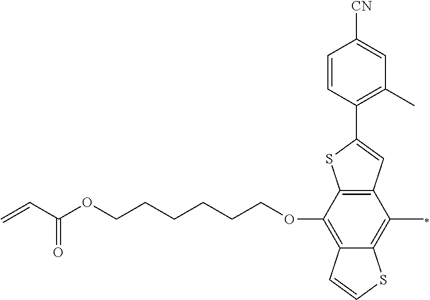 |
| (150) | 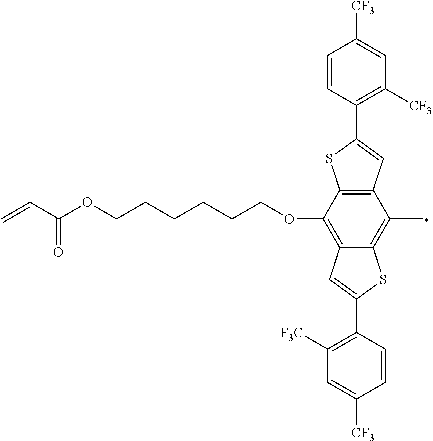 |
| (151) | 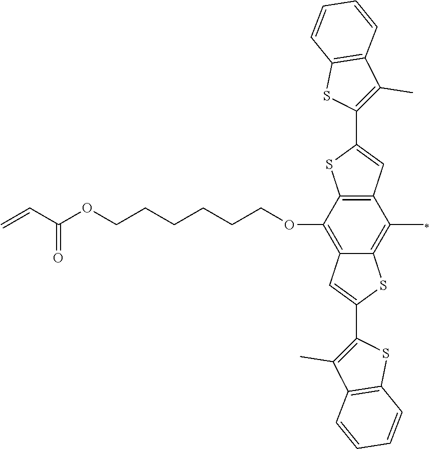 |
| (152) | 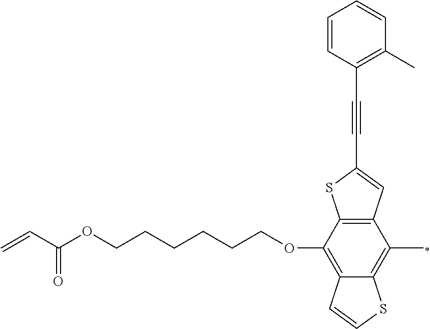 |
| (153) | 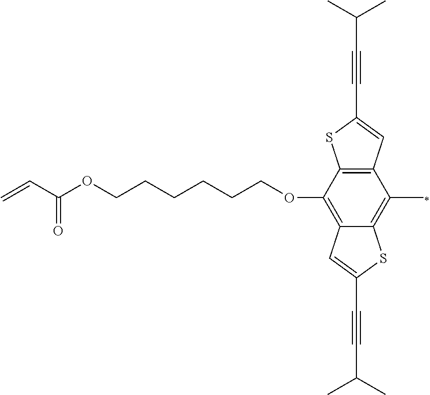 |
| (154) | 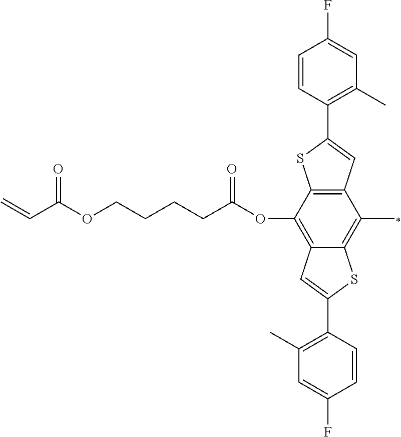 |
| (155) | 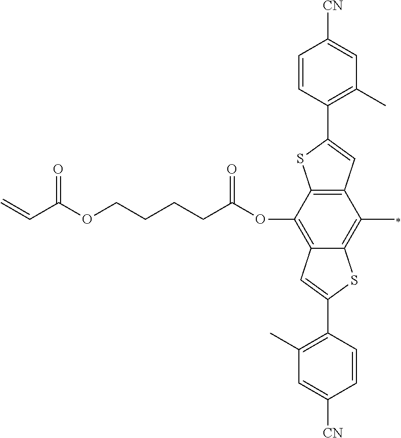 |
| (156) | 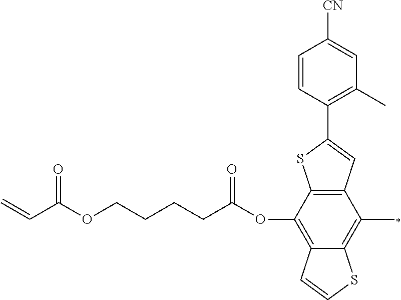 |
| (157) | 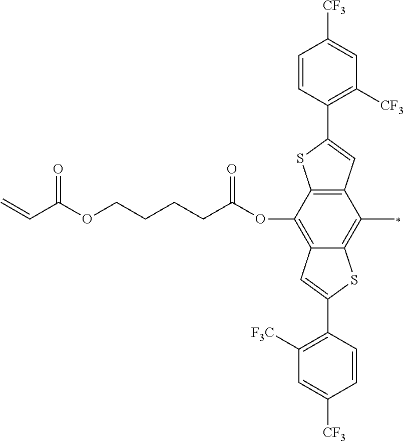 |
| (158) | 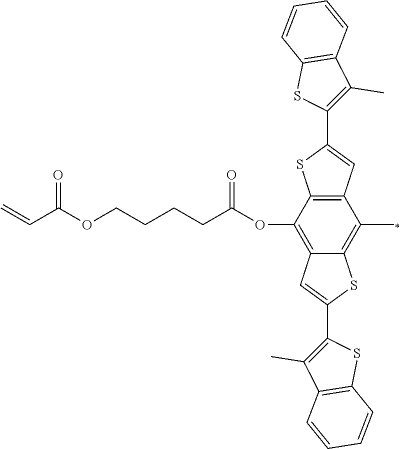 |
| (159) | 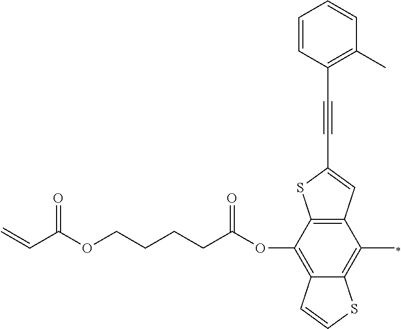 |
| (160) | 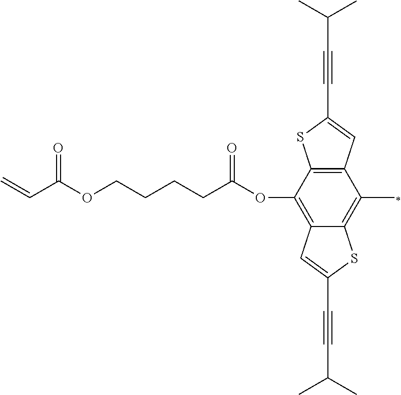 |

In the present invention, the reverse wavelength dispersibility of the compound represented by Formula (I-1) is high, and therefore, an optically anisotropic film having excellent reverse wavelength dispersibility and moisture-heat resistance can be formed even in a case of blending a small amount of the compound, as shown in a polymerizable composition of an embodiment of the present invention which will be described later.

### Polymerizable Composition

The polymerizable composition of the embodiment of the present invention is a polymerizable composition containing the compound represented by Formula (I-1) (specific compound) and a liquid crystal compound represented by Formula (II) which will be described later.

In addition, the content of the specific compound is 10% by mass or more, preferably 10% to 90% by mass, more preferably 10% to 50% by mass, still more preferably 20% to 50% by mass, and particularly preferably 30% to 50% by mass with respect to a total mass of the specific compound and the liquid crystal compound.

### Liquid Crystal Compound

The liquid crystal compound contained in the polymerizable composition of the embodiment of the present invention is a compound represented by Formula (II).

P¹¹-L¹-D¹⁵-(A¹¹)ₐ₁-D¹³-(G¹)_{g1}-D¹¹-[Ar³-D¹²]_{q1}-(G²)_{g2}-D¹⁴-(A¹²)ₐ₂-D¹⁶-L²-P¹² (II)

In Formula (II), a1, a2, g1, and g2 each independently represent 0 or 1. It should be noted that at least one of a1 or g1 represents 1, and at least one of a2 or g2 represents 1.

In addition, q1 represents 1 or 2.

Moreover, D¹¹, D¹², D¹³, D¹⁴, D¹⁵, and D¹⁶ each independently represent a single bond, or a divalent linking group consisting of —CO—, —O—, —S—, —C(═S)—, —CR¹R²—, —CR³═CR⁴—, —NR⁵—, or a combination of two or more of these groups, where R¹ to R⁵ each independently represent a hydrogen atom, a fluorine atom, or an alkyl group having 1 to 12 carbon atoms. It should be noted that in a case where q1 is 2, a plurality of D¹²'s may be the same as or different from each other.

In addition, G¹ and G² each independently represent an aromatic ring having 6 to 20 carbon atoms, which may have a substituent, or a divalent alicyclic hydrocarbon group having 5 to 20 carbon atoms, which may have a substituent, where one or more of —CH₂—'s constituting the alicyclic hydrocarbon group may be substituted with —O—, —S—, or —NH—.

Moreover, A¹¹ and A¹² each independently represent an aromatic ring having 6 to 20 carbon atoms, which may have a substituent, or a divalent alicyclic hydrocarbon group having 5 to 20 carbon atoms, which may have a substituent, where one or more of —CH₂—'s constituting the alicyclic hydrocarbon group may be substituted with —O—, —S—, or —NH—.

Furthermore, L¹ and L² each independently represent a single bond, a linear or branched alkylene group having 1 to 14 carbon atoms, or a divalent linking group in which one or more of —CH₂—'s constituting the linear or branched alkylene group having 1 to 14 carbon atoms are substituted with —O—, —S—, —NH—, —N(Q)—, or —CO—, where Q represents a substituent.

In addition, P¹¹ and P¹² each independently represent a monovalent organic group, where at least one of P¹¹ or P¹² represents a polymerizable group.

Moreover, Ar³ represents an aromatic ring having 6 to 20 carbon atoms, which may have a substituent, or a divalent alicyclic hydrocarbon group having 5 to 20 carbon atoms, which may have a substituent, where one or more of —CH₂—'s constituting the alicyclic hydrocarbon group may be substituted with —O—, —S—, or —NH—. It should be noted that in a case where q1 is 2, a plurality of Ar³'s may be the same as or different from each other.

In Formula (II), it is preferable that any of a1, a2, g1, and g2 is 1 for a reason that the polymerizable composition of the embodiment of the present invention is more likely to exhibit a liquid crystal state of a smectic phase.

In addition, it is preferable that both of a1 and a2 are 0 and both of g1 and g2 are 1 for a reason that the durability of an optically anisotropic film thus formed is improved.

In Formula (II), q1 is preferably 1.

In Formula (II), examples of the divalent linking group represented by one aspect of D¹¹, D¹², D¹³, D¹⁴, D¹⁵, and D¹⁶ include —CO—, —O—, —CO—O—, —C(═S)O—, —CR¹R²—, —CR¹R²—CR¹R²—, —O—CR¹R²—, —CR¹R²—O—CR¹R²—, —CO—O—CR¹R²—, —O—CO—CR¹R²—, —CR¹R²—O—CO—CR¹R²—, —CR¹R²—CO—O—CR¹R²—, —NR⁵—CR¹R²—, and —CO—NR⁵—. R¹, R², and R⁵ each independently represent a hydrogen atom, a fluorine atom, or an alkyl group having 1 to 12 carbon atoms.

Among these, any of —CO—, —O—, and —CO—O— is preferable.

In Formula (II), examples of the aromatic ring having 6 to 20 carbon atoms, represented by one aspect of G¹ and G², include aromatic hydrocarbon rings such as a benzene ring, a naphthalene ring, an anthracene ring, and a phenanthroline ring; and aromatic heterocyclic rings such as a furan ring, a pyrrole ring, a thiophene ring, a pyridine ring, a thiazole ring, and a benzothiazole ring. Among those, the benzene ring (for example, a 1,4-phenyl group) is preferable.

In Formula (II), the divalent alicyclic hydrocarbon group having 5 to 20 carbon atoms, represented by one aspect of G¹ and G², is preferably a 5- or 6-membered ring. In addition, the alicyclic hydrocarbon group may be saturated or unsaturated, but is preferably the saturated alicyclic hydrocarbon group. With regard to the divalent alicyclic hydrocarbon group represented by each of G¹ and G², reference can be made to, for example, the description in paragraph [0078] of JP2012-21068A, the contents of which are hereby incorporated by reference.

In the present invention, G¹ and G² in Formula (II) are each preferably a cycloalkane ring for a reason that the durability of an optically anisotropic film thus formed is improved.

Specific examples of the cycloalkane ring include a cyclohexane ring, a cyclopeptane ring, a cyclooctane ring, a cyclododecane ring, and a cyclodocosane ring.

Among those, the cyclohexane ring is preferable, a 1,4-cyclohexylene group is more preferable, and a trans-1,4-cyclohexylene group is still more preferable.

In addition, in G¹ and G² in Formula (II), examples of the substituent which may be contained in the aromatic ring having 6 to 20 carbon atoms or the divalent alicyclic hydrocarbon group having 5 to 20 carbon atoms include the above-described substituent E.

In Formula (II), examples of the aromatic ring having 6 to 20 or more carbon atoms, represented by one aspect of A¹¹ and A¹², include the same ones as those described for G¹ and G² in Formula (II).

In addition, in Formula (II), examples of the divalent alicyclic hydrocarbon group having 5 to 20 carbon atoms, represented by one aspect of A¹¹ and A¹², include the same ones as those described for G¹ and G² in Formula (II).

Moreover, examples of the substituent which may be contained in the aromatic ring having 6 to 20 carbon atoms or the divalent alicyclic hydrocarbon group having 5 to 20 carbon atoms in A¹¹ and A¹² include the above-described substituents a group E.

Suitable examples of the linear or branched alkylene group having 1 to 14 carbon atoms, represented by one aspect of L¹ and L², in Formula (II) include a methylene group, an ethylene group, a propylene group, a butylene group, a pentylene group, a hexylene group, a methylhexylene group, and a heptylene group. Furthermore, L¹ and L² may be a divalent linking group in which one or more of —CH₂—'s constituting the linear or branched alkylene group having 1 to 14 carbon atoms are substituted with —O—, —S—, —NH—, —N(Q)—, or —CO—, and examples of the substituent represented by Q include the above-described substituent E.

In Formula (II), examples of the monovalent organic group represented by each of P¹¹ and P¹² include the same ones as those described for P¹ and P² in Formula (I-1).

In Formula (II), examples of the polymerizable group represented by at least one of P¹¹ or P¹² include the same ones as those described for P¹ and P² in Formula (I-1).

In Formula (II), any of P¹¹ and P¹² in Formula (II) is preferably a polymerizable group, and more preferably an acryloyloxy group or a methacryloyloxy group for a reason that the durability of an optically anisotropic film thus formed is improved.

On the other hand, in Formula (II), examples of the aromatic ring having 6 to 20 or more carbon atoms, represented by one aspect of Ar³, include the same ones as those described in G¹ and G² in Formula (II).

In addition, in Formula (II), examples of the divalent alicyclic hydrocarbon group having 5 to 20 carbon atoms, represented by one aspect of Ar³, include the same ones as those described in G¹ and G² in Formula (II).

Furthermore, in Ar³, examples of the substituent which may be contained in the aromatic ring having 6 to 20 carbon atoms or the divalent alicyclic hydrocarbon group having 5 to 20 carbon atoms include the same ones as those of the substituent which may be contained in each of G¹ and G² in Formula (II).

In the present invention, from the viewpoint that the aligning properties are improved, the liquid crystal compound is preferably the compound in which Ar³ in Formula (II) represents any ring structure selected from the group consisting of the groups represented by Formulae (Ar-1) to (Ar-5) described above. Furthermore, in a case where Ar³ in Formula (II) represents any ring structure selected from the group consisting of the groups represented by Formulae (Ar-1) to (Ar-5) described above, * in Formulae (Ar-1) to (Ar-5) described above represents a bonding position to D¹¹ or D¹².

Examples of the compound represented by Formula (II) include the polymerizable compounds described in paragraphs [0019] to [0023] of JP2019-139222A; the polymerizable compounds described in paragraphs [0059] to [0061] of WO2019/160014A; the polymerizable compounds described in paragraph [0055] of WO2019/160016A; the compounds (1-1) to (1-19) represented by the following formulae; and compounds (2-1) to (2-5) represented by the following formulae. Furthermore, a group adjacent to the acryloyloxy group in the structure of the compound (1-14) represents a propylene group (a group obtained by substituting a methyl group with an ethylene group), and the compound (1-14) represents a mixture of regioisomers in which the positions of the methyl groups are different.

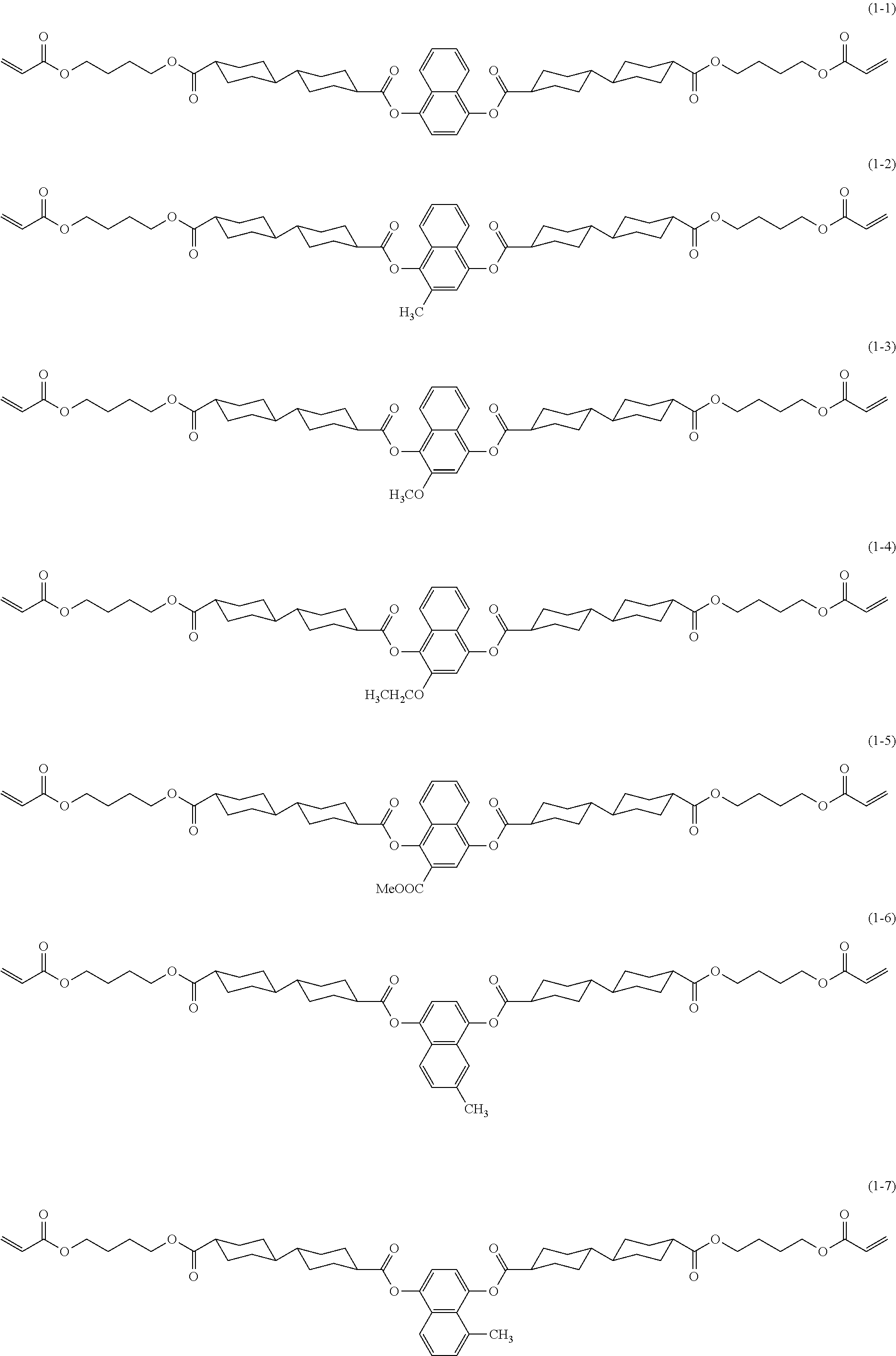

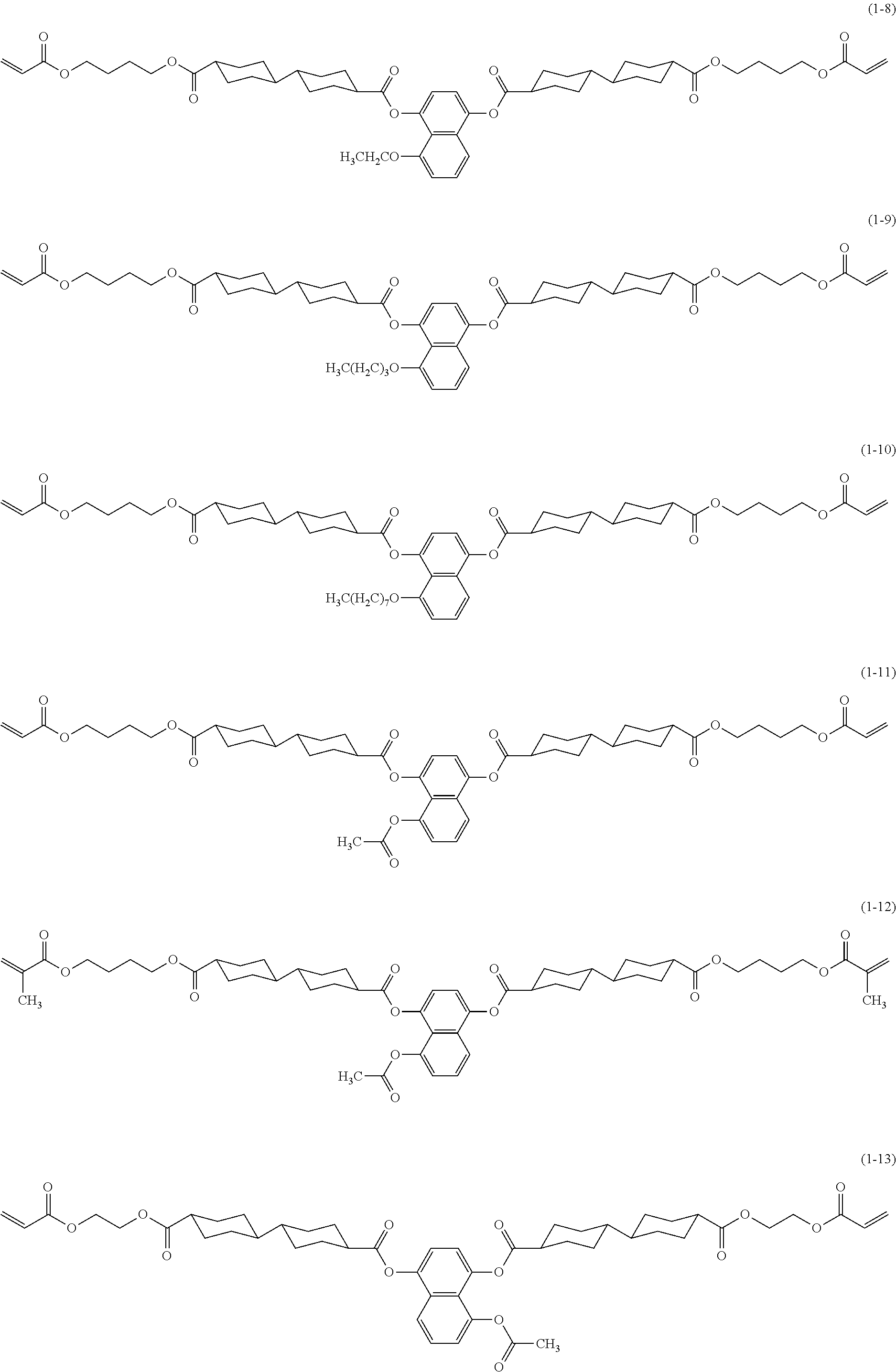

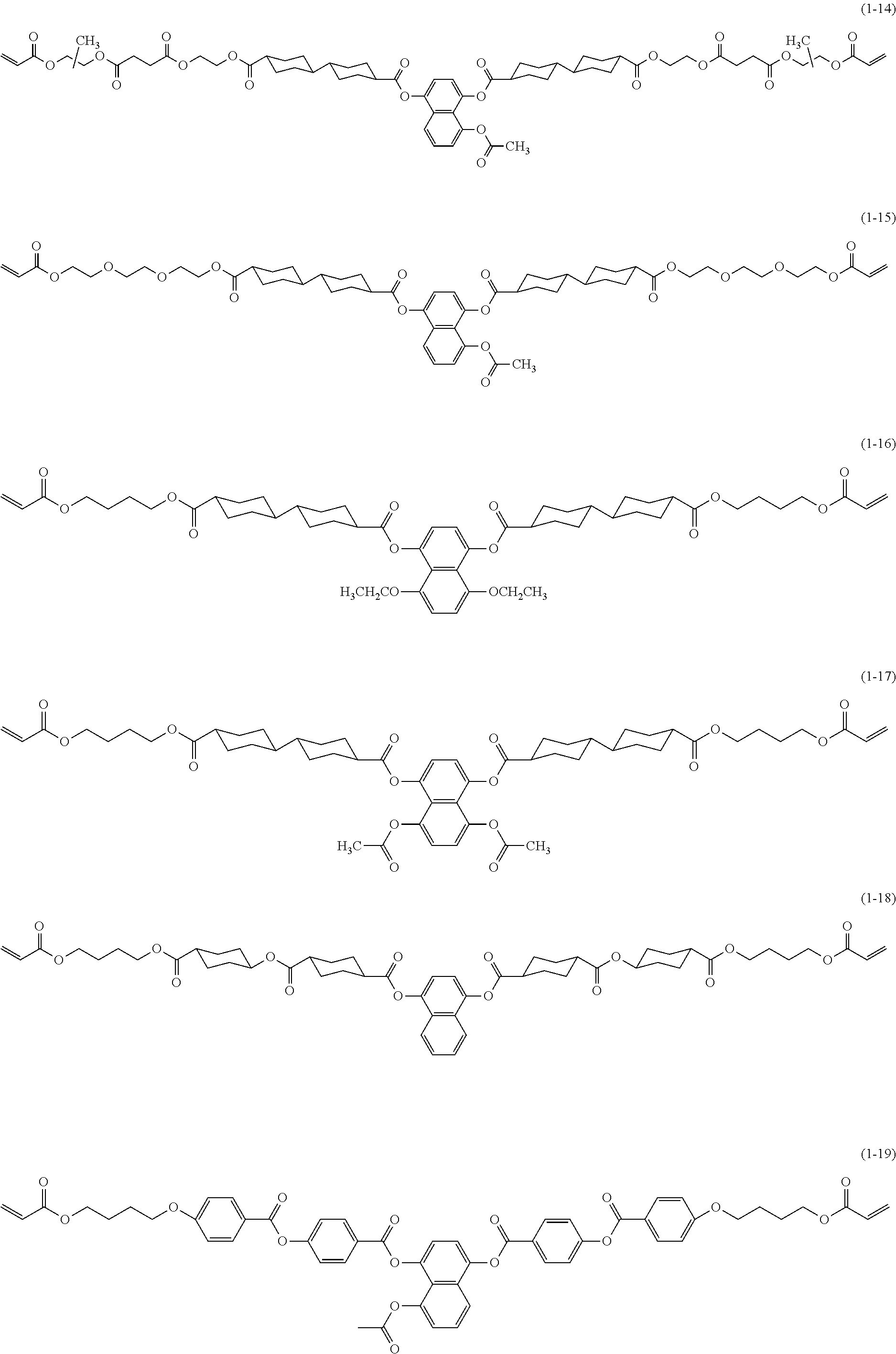

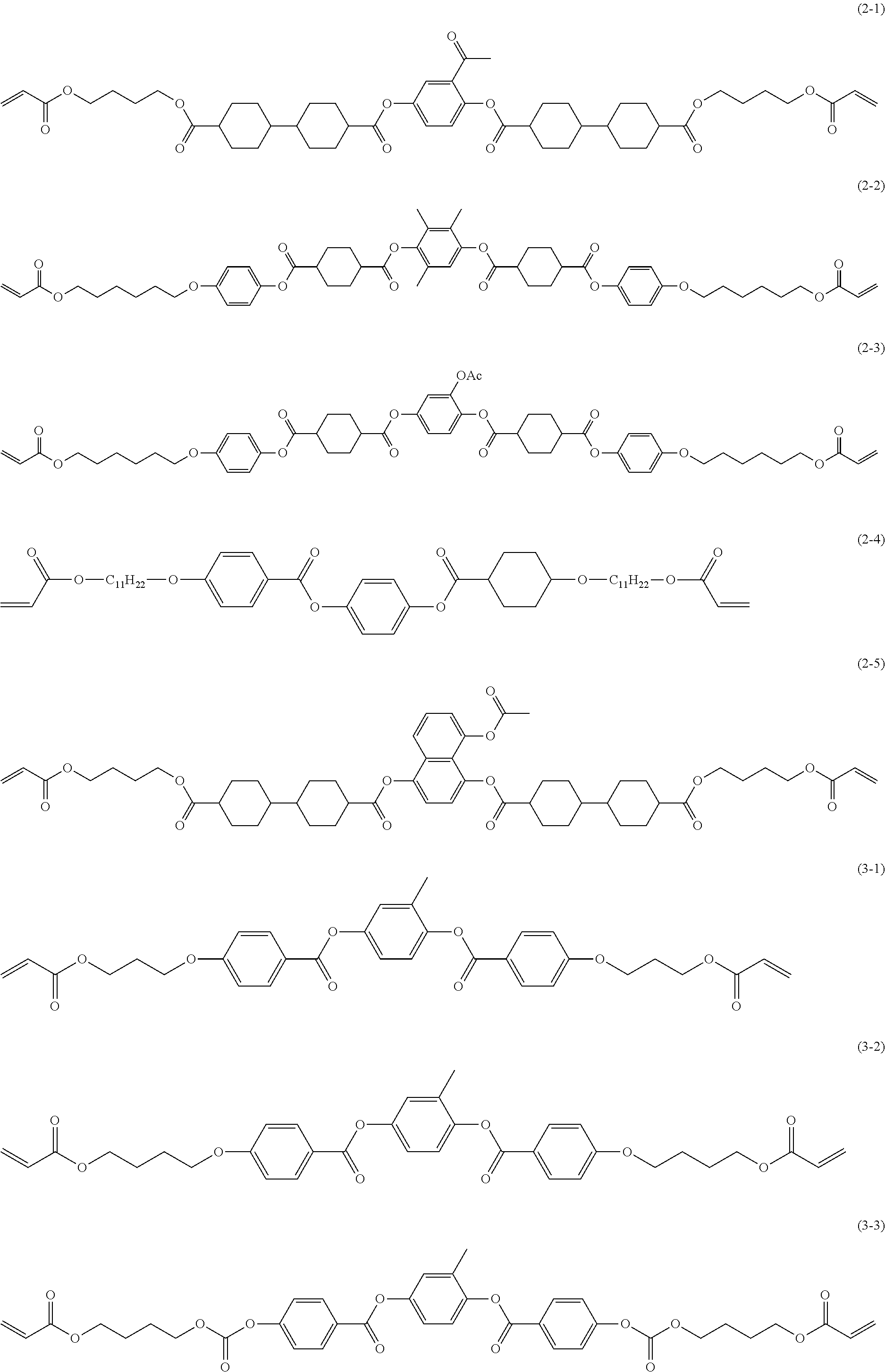

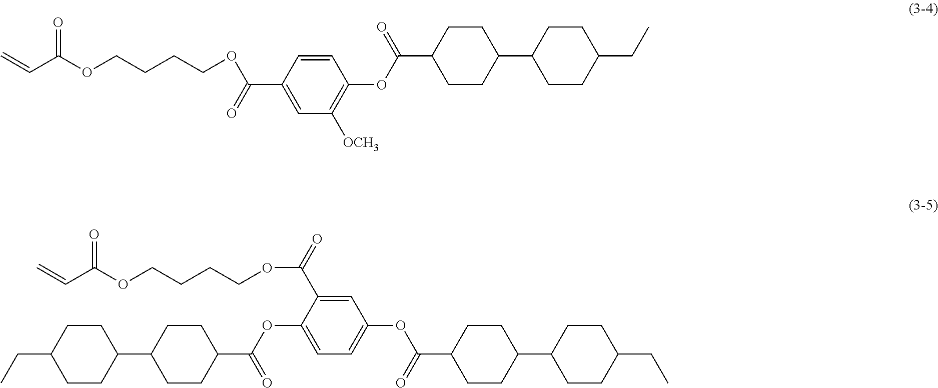

In addition, examples of the compound represented by Formula (II) include the compounds exhibiting smectic properties among the compounds represented by General Formula (1) described in JP2010-084032A (in particular, the compounds described in paragraph Nos. [0067] to [0073]), the compound represented by General Formula (II) described in JP2016-053709A (in particular, the compounds described in paragraph Nos. [0036] to [0043]), and the compounds represented by General Formula (1) described in JP2016-081035A (in particular, the compounds described in paragraph Nos. [0043] to [0055]).

Furthermore, suitable examples of the compound represented by Formula (II) include any of the compounds that exhibit smectic properties among the compounds represented by Formulae (1) to (22), and specifically compounds having side chain structures shown in Tables 1 to 3 below as K (side chain structure) in Formulae (1) to (22).

Moreover, in Tables 1 to 3 below, “*” shown in the side chain structure of K represents a bonding position to an aromatic ring.

In addition, in the side chain structures shown in 2-2 in Table 2 below and 3-2 in Table 3 below, a group adjacent to each of the acryloyloxy group and the methacryloyl group represents a propylene group (a group in which a methyl group is substituted with an ethylene group), and represents a mixture of regioisomers in which the positions of the methyl groups are different.

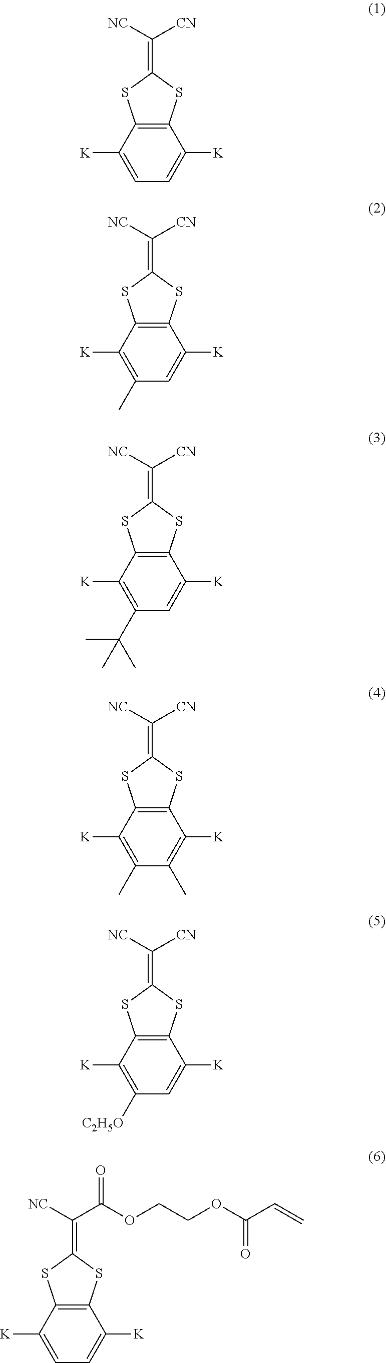

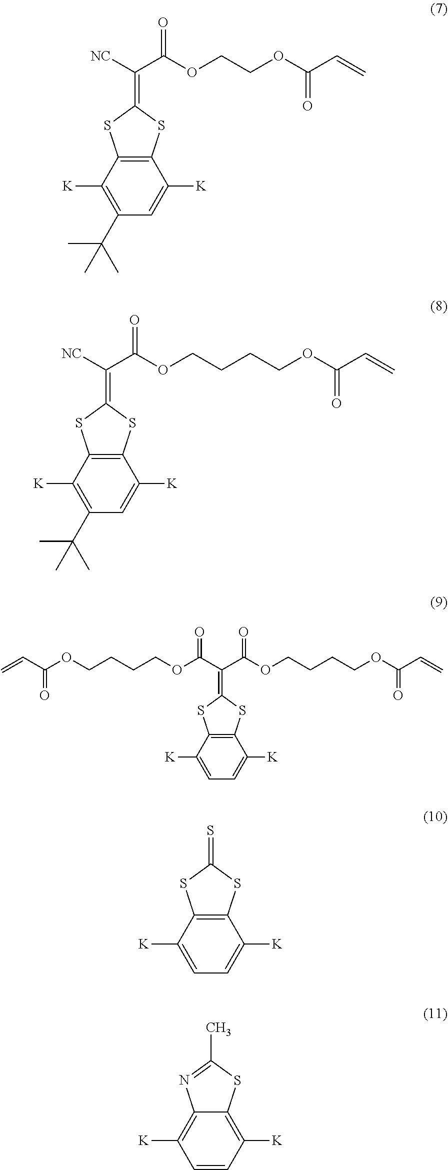

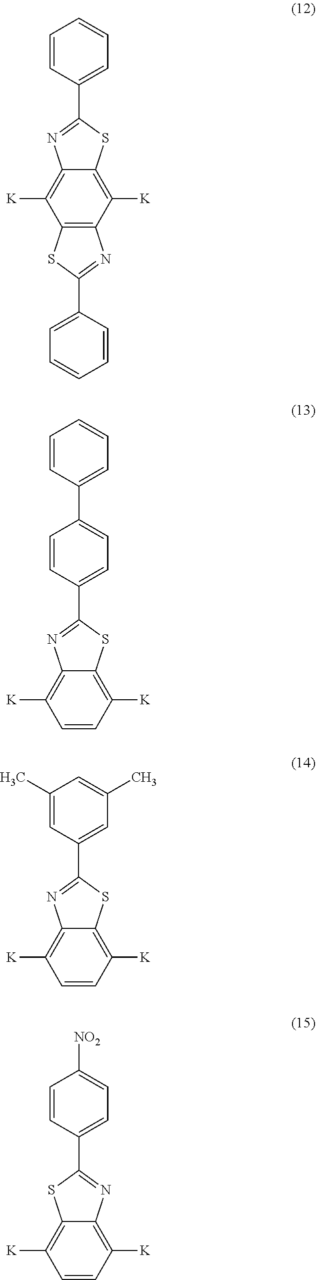

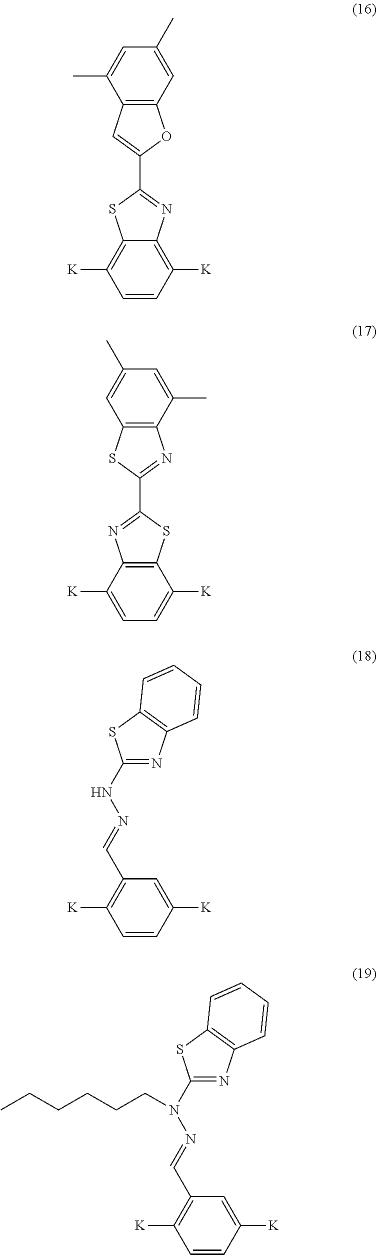

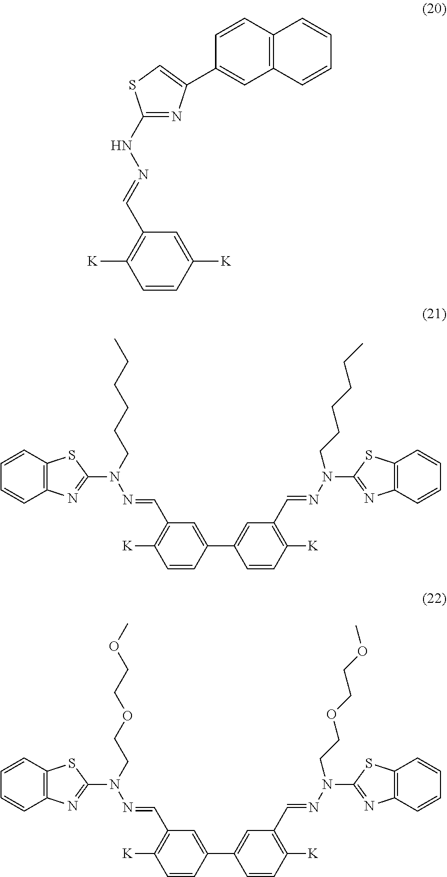

TABLE 1

| | K (side chain structure) |
|---|---|
| 1-1 | 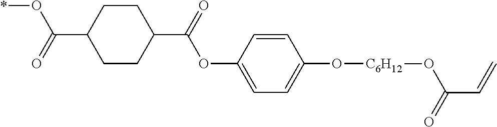 |
| 1-2 | 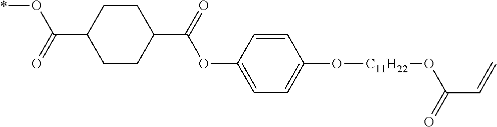 |
| 1-3 | 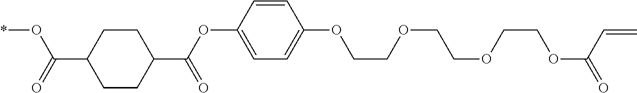 [SMILES: *OC(=O)C1CCC(C(=O)Oc2ccc(OCCOCCOCCOC(=O)C=C)cc2)CC1] |
| 1-4 | 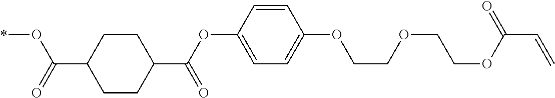 [SMILES: *OC(=O)C1CCC(C(=O)Oc2ccc(OCCOCCOC(=O)C=C)cc2)CC1] |
| 1-5 | 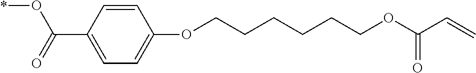 [SMILES: *OC(=O)c1ccc(OCCCCCCOC(=O)C=C)cc1] |
| 1-6 | 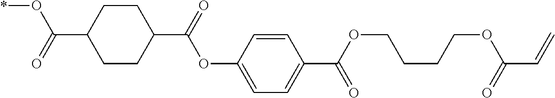 [SMILES: *OC(=O)C1CCC(C(=O)Oc2ccc(C(=O)OCCCCOC(=O)C=C)cc2)CC1] |

TABLE 2

| | K (side chain structure) |
|---|---|
| 2-1 | 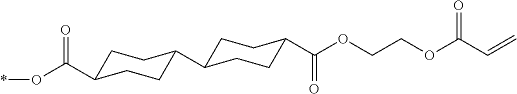 [SMILES: *OC(=O)C1CCC(C2CCC(C(=O)OCCOC(=O)C=C)CC2)CC1] |
| 2-2 | 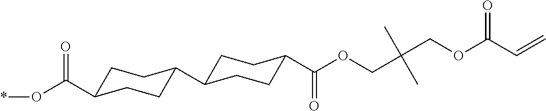 [SMILES: *OC(=O)C1CCC(C2CCC(C(=O)OCC(C)(C)COC(=O)C=C)CC2)CC1] |
| 2-3 | 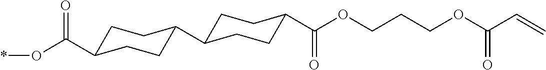 [SMILES: *OC(=O)C1CCC(C2CCC(C(=O)OCCCOC(=O)C=C)CC2)CC1] |
| 2-4 | 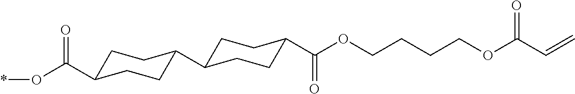 [SMILES: *OC(=O)C1CCC(C2CCC(C(=O)OCCCCOC(=O)C=C)CC2)CC1] |
| 2-5 | 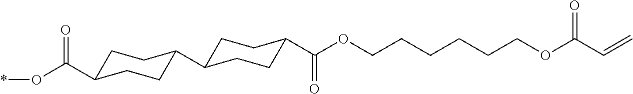 [SMILES: *OC(=O)C1CCC(C2CCC(C(=O)OCCCCCCOC(=O)C=C)CC2)CC1] |
| 2-6 | 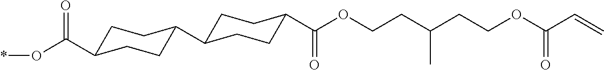 [SMILES: *OC(=O)C1CCC(C2CCC(C(=O)OCCC(C)CCOC(=O)C=C)CC2)CC1] |
| 2-7 | 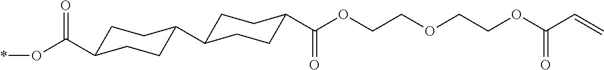 [SMILES: *OC(=O)C1CCC(C2CCC(C(=O)OCCOCCOC(=O)C=C)CC2)CC1] |
| 2-8 | 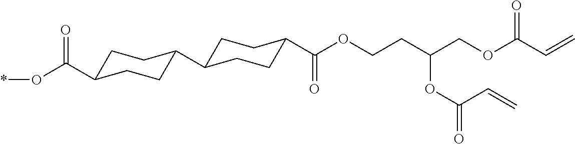 [SMILES: *OC(=O)C1CCC(C2CCC(C(=O)OCCC(COC(=O)C=C)OC(=O)C=C)CC2)CC1] |
| 2-9 | 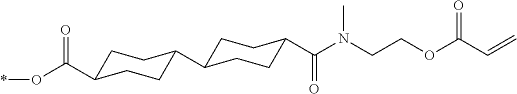 [SMILES: *OC(=O)C1CCC(C2CCC(C(=O)N(C)CCOC(=O)C=C)CC2)CC1] |
| 2-10 | 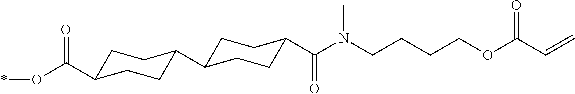 [SMILES: *OC(=O)C1CCC(C2CCC(C(=O)N(C)CCCCOC(=O)C=C)CC2)CC1] |
| 2-11 | 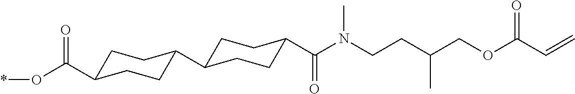 [SMILES: *OC(=O)C1CCC(C2CCC(C(=O)N(C)CCC(C)COC(=O)C=C)CC2)CC1] |
| 2-12 | 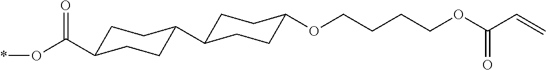 [SMILES: *OC(=O)C1CCC(C2CCC(OCCCCOC(=O)C=C)CC2)CC1] |
| 2-13 | 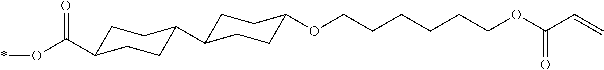 [SMILES: *OC(=O)C1CCC(C2CCC(OCCCCCCOC(=O)C=C)CC2)CC1] |
| 2-14 | 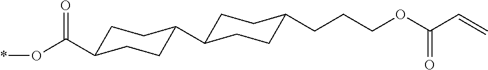 [SMILES: *OC(=O)C1CCC(C2CCC(CCCOC(=O)C=C)CC2)CC1] |

TABLE 3

| | K (side chain structure) |
|---|---|
| 3-1 | 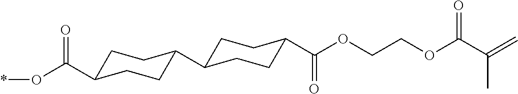 [SMILES: *OC(=O)C1CCC(C2CCC(C(=O)OCCOC(=O)C(=C)C)CC2)CC1] |
| 3-2 | 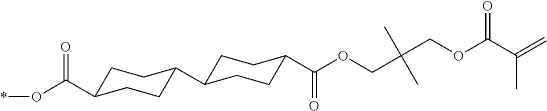 [SMILES: *OC(=O)C1CCC(C2CCC(C(=O)OCC(C)(C)COC(=O)C(=C)C)CC2)CC1] |
| 3-3 | 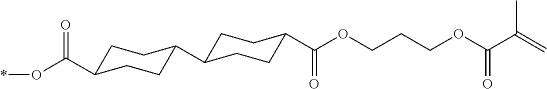 [SMILES: *OC(=O)C1CCC(C2CCC(C(=O)OCCCOC(=O)C(=C)C)CC2)CC1] |
| 3-4 | 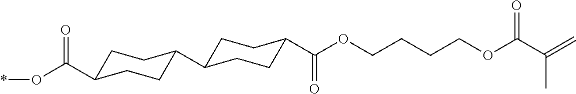 [SMILES: *OC(=O)C1CCC(C2CCC(C(=O)OCCCCOC(=O)C(=C)C)CC2)CC1] |
| 3-5 | 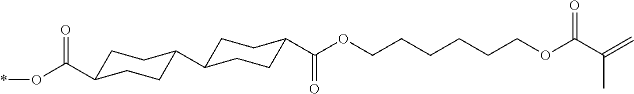 [SMILES: *OC(=O)C1CCC(C2CCC(C(=O)OCCCCCCOC(=O)C(=C)C)CC2)CC1] |
| 3-6 | 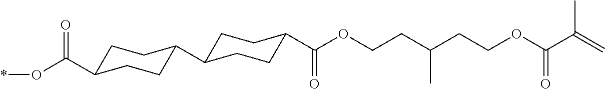 [SMILES: *OC(=O)C1CCC(C2CCC(C(=O)OCCC(C)CCOC(=O)C(=C)C)CC2)CC1] |
| 3-7 | 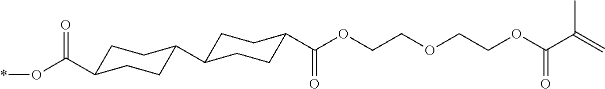 [SMILES: *OC(=O)C1CCC(C2CCC(C(=O)OCCOCCOC(=O)C(=C)C)CC2)CC1] |
| 3-8 | 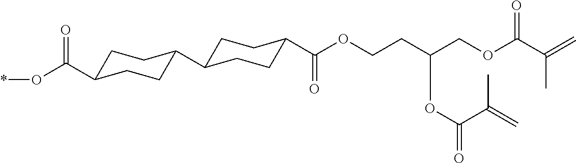 [SMILES: *OC(=O)C1CCC(C2CCC(C(=O)OCCC(COC(=O)C(=C)C)OC(=O)C(=C)C)CC2)CC1] |
| 3-9 | 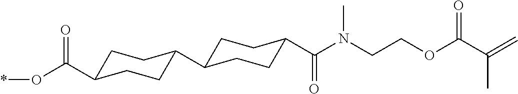 [SMILES: *OC(=O)C1CCC(C2CCC(C(=O)N(C)CCOC(=O)C(=C)C)CC2)CC1] |
| 3-10 | 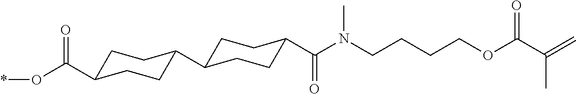 [SMILES: *OC(=O)C1CCC(C2CCC(C(=O)N(C)CCCCOC(=O)C(=C)C)CC2)CC1] |
| 3-11 | 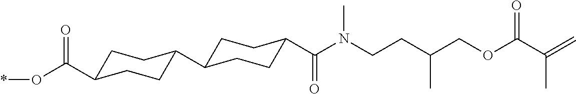 [SMILES: *OC(=O)C1CCC(C2CCC(C(=O)N(C)CCC(C)COC(=O)C(=C)C)CC2)CC1] |
| 3-12 | 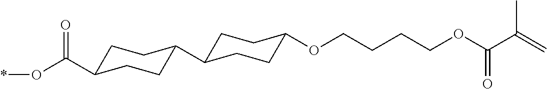 [SMILES: *OC(=O)C1CCC(C2CCC(OCCCCOC(=O)C(=C)C)CC2)CC1] |
| 3-13 | 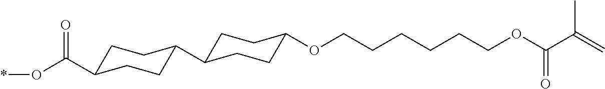 [SMILES: *OC(=O)C1CCC(C2CCC(OCCCCCCOC(=O)C(=C)C)CC2)CC1] |
| 3-14 | 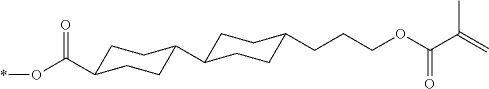 [SMILES: *OC(=O)C1CCC(C2CCC(CCCOC(=O)C(=C)C)CC2)CC1] |

### Other Polymerizable Compounds

The polymerizable composition of the embodiment of the present invention may include other polymerizable compounds having one or more polymerizable groups, in addition to the compound represented by Formula (I-1) and the liquid crystal compound represented by Formula (II).

Here, the polymerizable group contained in the other polymerizable compounds is not particularly limited, and examples thereof include the same groups as those described above for P¹ and P² in Formula (I-1). Among those, the acryloyloxy group or the methacryloyloxy group is preferable.

Examples of such other polymerizable compounds include the compounds described in paragraphs [0073] and [0074] of JP2016-053709A.

Furthermore, other examples of such other polymerizable compounds include the compounds represented by Formulae (M1), (M2), and (M3) described in paragraphs [0030] to [0033] of JP2014-077068A, and more specifically, the specific examples described in paragraphs [0046] to [0055] of the same publication.

In addition, as such other polymerizable compounds, the compounds having the structures of Formulae (1) to (3) described in JP2014-198814A can also be preferably used, and more specifically, examples of such other polymerizable compounds include the specific examples described in paragraphs [0020] to [0035], [0042] to [0050], [0056], and [0057] of the same publication.

In a case where the other polymerizable compounds are included, a content thereof is preferably less than 50% by mass, more preferably 40% by mass or less, and still more preferably 2% to 30% by mass with respect to a total mass of the compound represented by Formula (I-1) described above and the liquid crystal compound represented by Formula (II) described above.

### Polymerization Initiator

The polymerizable composition of the embodiment of the present invention preferably contains a polymerization initiator.

The polymerization initiator to be used is preferably a photopolymerization initiator capable of initiating a polymerization reaction upon irradiation with ultraviolet rays.

Examples of the photopolymerization initiator include a-carbonyl compounds (described in each of the specifications of U.S. Pat. Nos. 2,367,661A and 2,367,670A), acyloin ethers (described in the specification of U.S. Pat. No. 2,448,828A), α-hydrocarbon-substituted aromatic acyloin compounds (described in the specification of U.S. Pat. No. 2,722,512A), multinuclear quinone compounds (described in each of the specifications of U.S. Pat. Nos. 3,046,127A and 2,951,758A), combinations of a triarylimidazole dimer and a p-aminophenyl ketone (described in the specification of U.S. Pat. No. 3,549,367A), acridine and phenazine compounds (described in JP1985-105667A (JP-S60-105667A) and the specification of U.S. Pat. No. 4,239,850A), oxadiazole compounds (described in the specification of U.S. Pat. No. 4,212,970A), and acyl phosphine oxide compounds (described in JP1988-40799B (JP-S63-40799B), JP1993-29234B (JP-H05-29234B), JP1998-95788A (JP-H10-95788A), and JP1998-29997A (JP-H10-29997A)).

In addition, in the present invention, it is also preferable that the polymerization initiator is an oxime-type polymerization initiator, and specific examples of the polymerization

initiator include the initiators described in paragraphs to of WO2017/170443A.

### Solvent

It is preferable that the polymerizable composition of the embodiment of the present invention contains a solvent from the viewpoint of workability for forming an optically anisotropic film, and the like.

Specific examples of the solvent include ketone-based solvents (for example, acetone, 2-butanone, methyl isobutyl ketone, cyclohexanone, and cyclopentanone), ether-based solvents (for example, dioxane and tetrahydrofuran), cyclic amide-based solvents (for example, N-methylpyrrolidone, N-ethylpyrrolidone, and N,N′-dimethylimidazolidinone), aliphatic hydrocarbon-based solvents (for example, hexane), alicyclic hydrocarbon-based solvents (for example, cyclohexane), aromatic hydrocarbon-based solvents (for example, toluene, xylene, and trimethylbenzene), halogenated carbon-based solvents (for example, dichloromethane, dichloroethane, dichlorobenzene, and chlorotoluene), ester-based solvents (for example, methyl acetate, ethyl acetate, and butyl acetate), water, alcohol-based solvents (for example, ethanol, isopropanol, butanol, and cyclohexanol), cellosolve-based solvents (for example, methyl cellosolve and ethyl cellosolve), cellosolve acetate-based solvents, sulfoxide-based solvents (for example, dimethyl sulfoxide), and chain amide-based solvents (for example, dimethylformamide and dimethylacetamide), and these may be used alone or in combination of two or more kinds thereof.

In the present invention, among the above-described solvents, for a reason that the solubility of the specific compound of the embodiment of the present invention is good and the effect of suppressing precipitating properties is remarkable, at least one kind of solvent selected from the group consisting of the ketone-based solvents, the ether-based solvents, and the cyclic amide-based solvents is preferable.

### Leveling Agent

It is preferable that the polymerizable composition of the embodiment of the present invention contains a leveling agent from the viewpoint that the surface of an optically anisotropic film is maintained smooth and the alignment is easily controlled.

Such a leveling agent is preferably a fluorine-based leveling agent or a silicon-based leveling agent for a reason that it has a high leveling effect on the addition amount, and the leveling agent is more preferably a fluorine-based leveling agent from the viewpoint that it is less likely to cause bleeding (bloom or bleed).

Specific examples of the leveling agent include the compounds described in paragraphs [0079] to [0102] of JP2007-069471A, the compound represented by General Formula (I) described in JP2013-047204A (in particular, the compounds described in paragraphs [0020] to [0032]), the compound represented by General Formula (I) described in JP2012-211306A (in particular, the compounds described in paragraphs [0022] to [0029]), the liquid crystal alignment accelerator represented by General Formula (I) described in JP2002-129162A (in particular, the compounds described in paragraphs [0076] to [0078] and [0082] to [0084]), and the compounds represented by General Formulae (I), (II), and (III) described in JP2005-099248A (in particular, the compounds described in paragraphs [0092] to [0096]). Furthermore, the leveling agent may also function as an alignment control agent which will be described later.

### Alignment Control Agent

The polymerizable composition of the embodiment of the present invention can contain an alignment control agent, as desired.

With the alignment control agent, various alignment states such as homeotropic alignment (vertical alignment), tilt alignment, hybrid alignment, and cholesteric alignment can be formed, in addition to the homogeneous alignment, and specific alignment states can be controlled and achieved more uniformly and more accurately.

As an alignment control agent which accelerates the homogeneous alignment, for example, a low-molecular-weight alignment control agent or a high-molecular-weight alignment control agent can be used.

With regard to the low-molecular-weight alignment control agent, reference can be made to the description in, for example, paragraphs [0009] to [0083] of JP2002-20363A, paragraphs [0111] to [0120] of JP2006-106662A, and paragraphs [0021] to [0029] of JP2012-211306A, the contents of which are hereby incorporated by reference.

In addition, with regard to the high-molecular-weight alignment control agent, reference can be made to the description in, for example, paragraphs [0021] to [0057] of JP2004-198511A and paragraphs [0121] to [0167] of JP2006-106662A, the contents of which are hereby incorporated by reference.

Furthermore, examples of the alignment control agent that forms or accelerates the homeotropic alignment include a boronic acid compound and an onium salt compound, and specifically, reference can be made to the compounds described in paragraphs [0023] to [0032] of JP2008-225281A, paragraphs [0052] to [0058] of JP2012-208397A, paragraphs [0024] to [0055] of JP2008-026730A, paragraphs [0043] to [0055] of JP2016-193869A, and the like, the contents of which are hereby incorporated by reference.

On the other hand, the cholesteric alignment can be achieved by adding a chiral agent to the polymerizable composition of the embodiment of the present invention, and it is possible to control the direction of revolution of the cholesteric alignment by its chiral direction. Incidentally, it is possible to control the pitch of the cholesteric alignment in accordance with the alignment regulating force of the chiral agent.

In a case where an alignment control agent is contained, a content thereof is preferably 0.01% to 10% by mass, and more preferably 0.05% to 5% by mass with respect to the mass of the total solid content of the polymerizable composition. In a case where the content is within the range, it is possible to obtain an optically anisotropic film which has no precipitation or phase separation, alignment defects, or the like, and is uniform and highly transparent while achieving a desired alignment state.

These alignment control agents can further impart a polymerizable functional group, in particular, a polymerizable functional group which is polymerizable with a polymerizable compound or the polymerizable liquid crystal compound constituting the polymerizable composition of the embodiment of the present invention.

### Other Components

The polymerizable composition of the embodiment of the present invention may contain components other than the above-described components, and examples of such other components include a liquid crystal compound other than the above-described polymerizable compound or polymerizable liquid crystal compound, a surfactant, a tilt angle control agent, an alignment aid, a plasticizer, and a crosslinking agent.

### Optically Anisotropic Film

The optically anisotropic film of an embodiment of the present invention is an optically anisotropic film obtained by polymerizing the above-described polymerizable composition of the embodiment of the present invention.

Examples of a method for forming the optically anisotropic film include a method in which the above-described polymerizable composition of the embodiment of the present invention is used to cause a desired alignment state, which is then fixed by polymerization. In particular, in the present invention, an aspect in which the optically anisotropic film is formed after the above-described polymerizable composition of the embodiment of the present invention is prepared and then subjected to a step of storing the polymerizable composition is preferable.

Here, the polymerization conditions are not particularly limited, but in the polymerization by irradiation with light, ultraviolet rays are preferably used. The irradiation dose is preferably 10 mJ/cm² to 50 J/cm², more preferably 20 mJ/cm² to 5 J/cm², still more preferably 30 mJ/cm² to 3 J/cm², and particularly preferably 50 to 1,000 mJ/cm². In addition, the polymerization may be carried out under a heating condition in order to accelerate the polymerization reaction.

Furthermore, in the present invention, the optically anisotropic film can be formed on any of supports in the optical film of the embodiment of the present invention which will be described later or a polarizer in the polarizing plate of an embodiment of the present invention which will be described later.

The optically anisotropic film of the embodiment of the present invention preferably satisfies Formula (III).

$$0.5 < Re ( 450 ) / Re ( 550 ) < 1. \qquad ( III )$$

Here, in Formula (III), Re(450) represents an in-plane retardation of the optically anisotropic film at a wavelength of 450 nm, and Re(550) represents an in-plane retardation of the optically anisotropic film at a wavelength of 550 nm. In addition, in the present specification, in a case where the measurement wavelength of the retardation is not specified, the measurement wavelength is 550 nm.

The optically anisotropic film of the embodiment of the present invention is preferably a positive A-plate or a positive C-plate, and more preferably the positive A-plate.

Here, the positive A plate (A plate which is positive) and the positive C plate (C plate which is positive) are defined as follows.

In a case where a refractive index in a film in-plane slow axis direction (in a direction in which an in-plane refractive index is maximum) is defined as nx, a refractive index in an in-plane direction orthogonal to the in-plane slow axis is defined as ny, and a refractive index in a thickness direction is defined as nz, the positive A plate satisfies the relationship of Expression (A1) and the positive C plate satisfies the relationship of Expression (C1). In addition, the positive A plate has an Rth showing a positive value and the positive C plate has an Rth showing a negative value.

$$\begin{matrix}nx > ny \approx nz & Expression ( A1 ) \\ nz > nx \approx ny & Expression ( C 1 )\end{matrix}$$

Furthermore, the symbol, “≈”, encompasses not only a case where the both sides are completely the same as each other but also a case where the both sides are substantially the same as each other.

The expression, “substantially the same”, means that with regard to the positive A plate, for example, a case where (ny−nz)×d (in which d is the thickness of a film) is −10 to 10 nm, and preferably −5 to 5 nm is also included in “ny≈nz”, and a case where (nx−nz)×d is −10 to 10 nm, and preferably −5 to 5 nm is also included in “nx≈nz”. In addition, with regard to the positive C plate, for example, a case where (nx−ny)×d (in which d is the thickness of a film) is 0 to 10 nm, and preferably 0 to 5 nm is also included in “nx≈ny”.

In a case where the optically anisotropic film of the embodiment of the present invention is a positive A-plate, the Re(550) is preferably 100 to 180 nm, more preferably 120 to 160 nm, still more preferably 130 to 150 nm, and particularly preferably 130 to 140 nm, from the viewpoint that the optically anisotropic film functions as a λ/4 plate.

Here, the “λ/4 plate” is a plate having a λ/4 function, specifically, a plate having a function of converting a linearly polarized light at a certain specific wavelength into a circularly polarized light (or converting a circularly polarized light to a linearly polarized light).

### Optical Film

The optical film of the embodiment of the present invention is an optical film having the optically anisotropic film of the embodiment of the present invention.

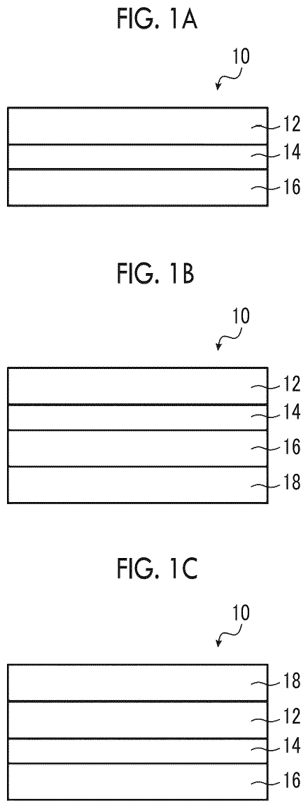

FIG. 1A, FIG. 1B, and FIG. 1C (these drawings are hereinafter simply referred to as “FIG. 1” unless it is necessary that they are particularly distinguished from each other) are each a schematic cross-sectional view showing an example of the optical film of the embodiment of the present invention.

Furthermore, FIG. 1 is a schematic view, and the thicknesses relationship, the positional relationship, and the like among the respective layers are not necessarily consistent with actual ones, and any of the support, the alignment film, and the hard coat layer shown in FIG. 1 are optional constitutional members.

An optical film 10 shown in FIG. 1 has a support 16, an alignment film 14, and an optically anisotropic film 12 in this order.

In addition, the optical film 10 may have a hard coat layer 18 on the side of the support 16 opposite to the side on which the alignment film 14 is provided as shown in FIG. 1B, and may have the hard coat layer 18 on the side of the optically anisotropic film 12 opposite to the side on which the alignment film 14 is provided as shown in FIG. 1C.

Hereinafter, various members used for the optical film of the embodiment of the present invention will be described in detail.

### Optically Anisotropic Film

The optically anisotropic film which the optical film of the embodiment of the present invention has is the above-described optically anisotropic film of the embodiment of the present invention.

In the optical film of the embodiment of the present invention, the thickness of the optically anisotropic film is not particularly limited, but is preferably 0.1 to 10 μm, and more preferably 0.5 to 5 μm.

### Support

The optical film of the embodiment of the present invention may have a support as a base material for forming an optically anisotropic film as described above.

Such a support is preferably transparent, and specifically, it preferably has a light transmittance of 80% or more.

Examples of such a support include a glass substrate and a polymer film, and examples of the material for the polymer film include cellulose-based polymers; acrylic polymers having an acrylic ester polymer such as polymethyl methacrylate and a lactone ring-containing polymer; thermoplastic norbornene-based polymers; polycarbonate-based polymers; polyester-based polymers such as polyethylene terephthalate and polyethylene naphthalate; styrene-based polymers such as polystyrene and an acrylonitrile-styrene copolymer (AS resin); polyolefin-based polymers such as polyethylene, polypropylene, and an ethylene-propylene copolymer; vinyl chloride-based polymers; amide-based polymers such as nylon and aromatic polyamide; imide-based polymers; sulfone-based polymers; polyether sulfone-based polymers; polyether ether ketone-based polymers; polyphenylene sulfide-based polymers; vinylidene chloride-based polymers; vinyl alcohol-based polymers; vinyl butyral-based polymers; arylate-based polymers; polyoxymethylene-based polymers; epoxy-based polymers; and polymers obtained by mixing these polymers.

In addition, an aspect in which a polarizer which will be described later may also function as such a support is also available.

In the present invention, the thickness of the support is not particularly limited, but is preferably 5 to 60 μm, and more preferably 5 to 30 μm.

### Alignment Film

In a case where the optical film of the embodiment of the present invention has any of the above-described supports, it is preferable that the optical film has an alignment film between the support and the optically anisotropic film. Furthermore, an aspect in which the above-described support may also function as an alignment film is also available.

The alignment film generally has a polymer as a main component. The materials for the polymer material for an alignment film are described in many documents, and many commercially available products can be used.

The polymer material used in the present invention is preferably a polyvinyl alcohol or a polyimide, or a derivative thereof. Particularly, a modified or non-modified polyvinyl alcohol is preferable.

Examples of the alignment film that can be used in the present invention include the alignment films described for Line 24 on Page 43 to Line 8 on Page 49 of WO01/88574A; the modified polyvinyl alcohols described in paragraphs [0071] to [0095] of JP3907735B; and the liquid crystal alignment film formed by a liquid crystal alignment agent described in JP2012-155308A.

In the present invention, for a reason that it is possible to prevent deterioration in the surface condition by avoiding a contact with the surface of an alignment film upon formation of the alignment film, a photoalignment film is also preferably used as the alignment film.

The photoalignment film is not particularly limited, but the polymer materials such as a polyamide compound and a polyimide compound, described in paragraphs [0024] to [0043] of WO2005/096041A; the liquid crystal alignment film formed by a liquid crystal alignment agent having a photoalignment group, described in JP2012-155308A; LPP-JP265CP, trade name, manufactured by Rolic Technologies Ltd.; or the like can be used.

In addition, in the present invention, the thickness of the alignment film is not particularly limited, but from the viewpoint of forming an optically anisotropic film having a homogeneous film thickness by alleviating the surface roughness that can be present on the support, the thickness is preferably 0.01 to 10 μm, more preferably 0.01 to 1 μm, and still more preferably 0.01 to 0.5 μm.

### Hard Coat Layer

It is preferable that the optical film of the embodiment of the present invention has a hard coat layer in order to impart physical strength to the film. Specifically, the optical film may have the hard coat layer on the side of the support opposite to the side on which the alignment film is provided (see FIG. 1B) or the optical film may have the hard coat layer on the side of the optically anisotropic film opposite to the side on which the alignment film is provided (see FIG. 1C).

As the hard coat layer, those described in paragraphs [0190] to [0196] of JP2009-98658A can be used.

### Other Optically Anisotropic Films

The optical film of the embodiment of the present invention may have other optically anisotropic films, in addition to the optically anisotropic film of the embodiment of the present invention.

That is, the optical film of the embodiment of the present invention may have a laminated structure having the optically anisotropic film of the embodiment of the present invention and other optically anisotropic films.

Such other optically anisotropic films are not particularly limited as long as the optically anisotropic films are obtained using the above-described liquid crystal compound or other polymerizable compounds (in particular, a liquid crystal compound) without blending the compound represented by Formula (I-1) and the liquid crystal compound represented by (I-2) described above.

Here, the liquid crystal compounds can be generally classified into a rod-shaped type and a disk-shaped type according to the shape thereof. Furthermore, each liquid crystal compound may be either of a low-molecular-weight type or of a high-molecular-weight type. The term, high-molecular-weight, generally refers to having a degree of polymerization of 100 or more (Polymer Physics-Phase Transition Dynamics, by Masao Doi, page 2, published by Iwanami Shoten, Publishers, 1992). In the present invention, any of the liquid crystal compounds can be used, but the rod-like liquid crystal compound or the discotic liquid crystal compound (disk-like liquid crystal compound) is preferably used. Two or more kinds of the rod-shaped liquid crystal compounds, two or more kinds of the disk-shaped liquid crystal compounds, or a mixture of the rod-shaped liquid crystal compound and the disk-shaped liquid crystal compound may be used. In order to fix the above-described liquid crystal compound, it is more preferable that the liquid crystal compound is formed of a rod-shaped liquid crystal compound or disk-shaped liquid crystal compound having a polymerizable group, and it is still more preferable that the liquid crystal compound has two or more polymerizable groups in one molecule. In the case of a mixture of two or more kinds of the liquid crystal compounds, at least one kind of the liquid crystal compound preferably has two or more polymerizable groups in one molecule.

As the rod-like liquid crystal compound, for example, the rod-like liquid crystal compounds described in claim 1 of JP1999-513019A (JP-H11-513019A) or paragraphs [0026] to [0098] of JP2005-289980A can be preferably used, and as the discotic liquid crystal compound, for example, the discotic liquid crystal compounds described in paragraphs [0020] to [0067] of JP2007-108732A and paragraphs to of JP2010-244038A can be preferably used, but the rod-like liquid crystal compounds and the discotic liquid crystal compounds are not limited thereto.

### Ultraviolet Absorbing Agent

The optical film of the embodiment of the present invention preferably includes an ultraviolet (UV) absorber, taking an effect of external light (particularly ultraviolet rays) into consideration.

The ultraviolet absorber may be contained in the optically anisotropic film of the embodiment of the present invention or may also be contained in a member other than an optically anisotropic film constituting the optical film of the embodiment of the present invention. Suitable examples of the member other than the optically anisotropic film include a support.

As the ultraviolet absorber, any one of ultraviolet absorbers known in the related art, which can express ultraviolet absorptivity, can be used. Among such the ultraviolet absorbers, a benzotriazole-based or hydroxyphenyltriazine-based ultraviolet absorber is preferably used from the viewpoint that it has high ultraviolet absorptivity and ultraviolet absorbing ability (ultraviolet-shielding ability) used for an image display device is obtained.

In addition, in order to broaden ultraviolet absorbing ranges, two or more kinds of ultraviolet absorbers having different maximum absorption wavelengths can be used in combination.

Specific examples of the ultraviolet absorber include the compounds described in paragraphs [0258] and [0259] of JP2012-18395A and the compounds described in paragraphs [0055] to [0105] of JP2007-72163A.

In addition, as a commercially available product thereof, for example, Tinuvin 400, Tinuvin 405, Tinuvin 460, Tinuvin 477, Tinuvin 479, and Tinuvin 1577 (all manufactured by BASF), or the like can be used.

### Polarizing Plate

A polarizing plate of an embodiment of the present invention has the above-described optical film of the embodiment of the present invention and a polarizer.

Furthermore, in a case where the above-described optically anisotropic film of the embodiment of the present invention is a λ/4 plate (positive A-plate), the polarizing plate of the embodiment of the present invention can be used as a circularly polarizing plate.

In addition, in a case where the above-described optically anisotropic film of the embodiment of the present invention is a λ/4 plate (positive A-plate), an angle between the slow axis of the λ/4 plate and the absorption axis of a polarizer which will be described later is preferably 30° to 60°, more preferably 40° to 50°, still more preferably 42° to 48°, and particularly preferably 45° in the polarizing plate of the embodiment of the present invention.

Here, the “slow axis” of the λ/4 plate means a direction in which the refractive index in the plane of the λ/4 plate is maximum, and the “absorption axis” of the polarizer means a direction in which the absorbance is highest.

### Polarizer

A polarizer contained in a polarizing plate of the embodiment of the present invention is not particularly limited as long as it is a member having a function of converting light into specific linearly polarized light, and an absorptive type polarizer and a reflective type polarizer, which are known in the related art, can be used.

An iodine-based polarizer, a dye-based polarizer using a dichroic dye, a polyene-based polarizer, or the like is used as the absorptive type polarizer. The iodine-based polarizer and the dye-based polarizer are classified into a coating type polarizer and a stretching type polarizer, any of which can be applied, but a polarizer which is manufactured by allowing polyvinyl alcohol to adsorb iodine or a dichroic dye and performing stretching is preferable.

In addition, examples of a method of obtaining a polarizer by carrying out stretching and dying in a state of a laminated film in which a polyvinyl alcohol layer is formed on a base material include the methods disclosed in JP5048120B, JP5143918B, JP4691205B, JP4751481B, and JP4751486B, and known technologies relating to these polarizers can also be preferably used.

A polarizer in which thin films having different birefringence are laminated, a wire grid-type polarizer, a polarizer having a combination of a cholesteric liquid crystal having a selective reflection range, and a ¼ wavelength plate, or the like is used as the reflective type polarizer.

Among those, a polarizer including a polyvinyl alcohol-based resin (a polymer including —CH₂—CHOH— as a repeating unit, in particular, at least one selected from the group consisting of a polyvinyl alcohol and an ethylene-vinyl alcohol copolymer) is preferable from the viewpoint that it has more excellent adhesiveness.

In the present invention, the thickness of the polarizer is not particularly limited, but is preferably 3 μm to 60 μm, more preferably 5 μm to 30 μm, and still more preferably 5 μm to 15 μm.

### Pressure Sensitive Adhesive Layer

The polarizing plate of the embodiment of the present invention may have a pressure sensitive adhesive layer arranged between the optically anisotropic film in the optical film of the embodiment of the present invention and the polarizer.

The pressure sensitive adhesive layer used for lamination of the optically anisotropic film and the polarizer represents, for example, a substance in which a ratio (tanδ-G″/G′) between a storage elastic modulus G′ and a loss elastic modulus G″, each measured with a dynamic viscoelastometer, is 0.001 to 1.5, and examples thereof include a so-called pressure sensitive adhesive or a readily creepable substance. Examples of the pressure sensitive adhesive that can be used in the present invention include a polyvinyl alcohol-based pressure sensitive adhesive, but the pressure sensitive adhesive is not limited thereto.

### Image Display Device

An image display device of an embodiment of the present invention is an image display device having the optical film of the embodiment of the present invention or the polarizing plate of the embodiment of the present invention.

A display element used in the image display device of the embodiment of the present invention is not particularly limited, and examples thereof include a liquid crystal cell, an organic electroluminescent (hereinafter simply referred to as “EL”) display panel, and a plasma display panel.

Among those, the liquid crystal cell and the organic EL display panel are preferable, and the liquid crystal cell is more preferable. That is, as the image display device of the embodiment of the present invention, a liquid crystal display device using a liquid crystal cell as a display element or an organic EL display device using an organic EL display panel as a display element is preferable, and the liquid crystal display device is more preferable.

### Liquid Crystal Display Device

A liquid crystal display device which is an example of the image display device of the embodiment of the present invention is a liquid crystal display device having the above-described polarizing plate of the embodiment of the present invention and a liquid crystal cell.

In addition, in the present invention, it is preferable that the polarizing plate of the embodiment of the present invention is used as the polarizing plate of the front side, and it is more preferable that the polarizing plate of the embodiment of the present invention is used as the polarizing plates on the front and rear sides, among the polarizing plates provided on the both sides of the liquid crystal cell.

Hereinafter, the liquid crystal cell constituting the liquid crystal display device will be described in detail.

### Liquid Crystal Cell

The liquid crystal cell used for the liquid crystal display device is preferably in a vertical alignment (VA) mode, an optically compensated bend (OCB) mode, an in-plane-switching (IPS) mode, a fringe-field-switching (FFS) mode, or a twisted nematic (TN) mode, but is not limited thereto.

In a TN-mode liquid crystal cell, rod-shaped liquid crystal molecules are substantially horizontally aligned and are twist-aligned at 60° to 120° during no voltage application thereto. A TN-mode liquid crystal cell is most often used in a color TFT liquid crystal display device and described in numerous documents.

In a VA-mode liquid crystal cell, rod-shaped liquid crystal molecules are substantially vertically aligned during no voltage application thereto. Examples of the VA-mode liquid crystal cell include (1) a VA-mode liquid crystal cell in the narrow sense of the word, in which rod-shaped liquid crystal molecules are substantially vertically aligned during no voltage application thereto, but are substantially horizontally aligned during voltage application thereto (described in JP1990-176625A (JP-H02-176625A)), (2) an MVA-mode liquid crystal cell in which the VA-mode is multi-domained for viewing angle enlargement (described in SID97, Digest of Tech. Papers (preprint), 28 (1997) 845), (3) a liquid crystal cell in a mode (n-ASM mode) in which rod-shaped liquid crystal molecules are substantially vertically aligned during no voltage application thereto and are twistedly multi-domain-aligned during voltage application thereto (described in Seminar of Liquid Crystals of Japan, Papers (preprint), 58-59 (1998)), and (4) a survival-mode liquid crystal cell (announced in LCD International 98). In addition, the liquid crystal cell may be of any of a patterned vertical alignment (PVA) type, an optical alignment type, and a polymer-sustained alignment (PSA). Details of these modes are specifically described in JP2006-215326A and JP2008-538819A.

In an IPS-mode liquid crystal cell, rod-shaped liquid crystal molecules are aligned substantially parallel with respect to a substrate, and application of an electric field parallel to the substrate surface causes the liquid crystal molecules to respond planarly. The IPS-mode displays black in a state where no electric field is applied and a pair of upper and lower polarizing plates have absorption axes which are orthogonal to each other. A method of improving the viewing angle by reducing light leak during black display in an oblique direction using an optical compensation sheet is disclosed in JP1998-54982A (JP-H10-54982A), JP1999-202323A (JP-H11-202323A), JP1997-292522A (JP-H09-292522A), JP1999-133408A (JP-H11-133408A), JP1999-305217A (JP-H11-305217A), JP1998-307291A (JP-H10-307291A), and the like.

### Organic EL Display Device

Suitable examples of the organic EL display device which is an example of the image display device of the embodiment of the present invention include an aspect which includes, from the visible side, a polarizer, a λ/4 plate (a positive A-plate) including the optically anisotropic film of the embodiment of the present invention, and an organic EL display panel in this order.

Furthermore, the organic EL display panel is a display panel composed of an organic EL device in which an organic light emitting layer (organic electroluminescent layer) is sandwiched between electrodes (between a cathode and an anode). The configuration of the organic EL display panel is not particularly limited but a known configuration is adopted.

### EXAMPLES

Hereinafter, the present invention will be described in more detail with reference to Examples. The materials, the amounts of materials used, the proportions, the treatment details, the treatment procedure, and the like shown in Examples below can be appropriately modified as long as the modifications do not depart from the spirit of the present invention. Therefore, the scope of the present invention should not be construed as being limited to Examples shown below.

### Synthesis of Compound (96)

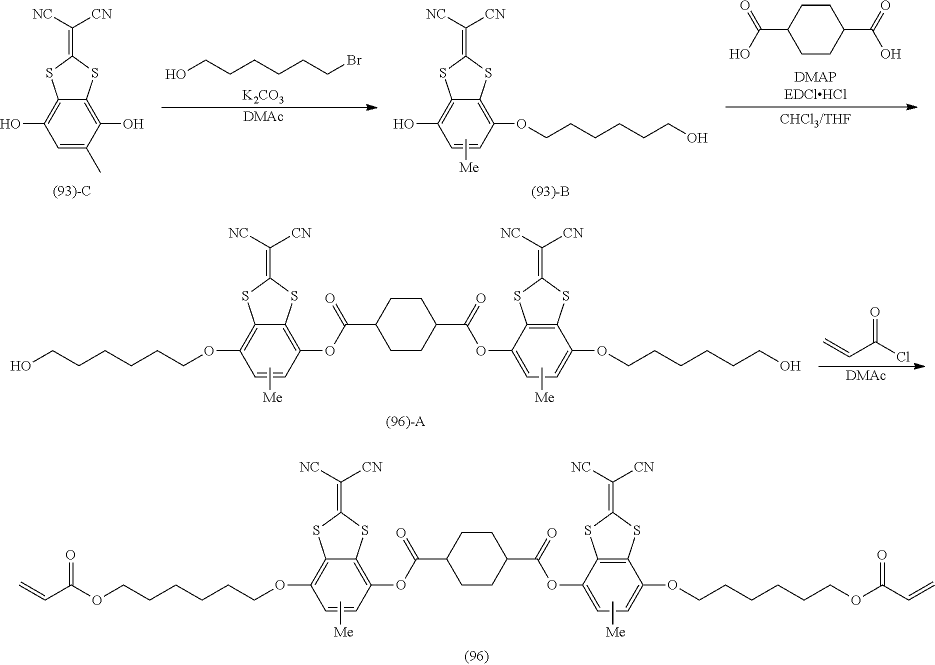

In a nitrogen atmosphere, 1.31 g of a raw material compound (93)-C represented by Formula (93)-C and 10 mL of N,N-dimethylacetamide were added to a 300 mL three-necked flask, 0.91 g of 6-bromo-1-hexanol was added dropwise thereto under stirring at room temperature, and 1.0 g of potassium carbonate was added to the mixture. After stirring at 80° C. for 4 hours, the mixture was cooled to room temperature, 100 ml of water and 100 mL of ethyl acetate were added thereto, and the mixture was stirred at room temperature. The organic layer recovered by liquid separation was concentrated, 100 mL of 1 M diluted hydrochloric acid and 100 mL of ethyl acetate were added thereto, and the mixture was stirred at room temperature. The organic layer recovered by liquid separation was concentrated and then purified by silica gel column chromatography (developing solvent: hexane/ethyl acetate) to obtain 0.50 g of a compound (93)-B represented by Formula (93)-B.

0.50 g of the compound (93)-B and 5 mL of tetrahydrofuran (THF) were added to a 100 mL three-neck flask, 0.10 g of cyclohexyl-1,4-dicarboxylic acid, 0.01 g of 4-dimethylaminopyridine, 0.35 g of 1-(3-dimethylaminopropyl)-3-ethylcarbodiimide hydrochloride, and 5 mL of chloroform were added thereto under stirring at room temperature, and the mixture was stirred at room temperature for 12 hours. The reaction solution was concentrated and purified by silica gel column chromatography (developing solvent: hexane/ethyl acetate) to obtain 0.38 g of a compound (96)-A represented by Formula (96)-A.

0.38 g of the compound (96)-A and 5 mL of N,N-dimethylacetamide were added to a 100 mL three-neck flask, and the mixture was stirred under ice cooling in a nitrogen atmosphere. 0.3 mL of acryloyl chloride was added dropwise thereto, the temperature was raised to room temperature, and the mixture was further stirred for 1 hour. 10 mL of water and 20 mL of ethyl acetate were added to this reaction solution, and the organic layer obtained by extraction and liquid separation was concentrated and then purified by silica gel column chromatography (developing solvent: hexane/ethyl acetate) and crystallization (chloroform/methanol) to obtain 0.12 g of a compound (96) represented by Formula (96).

The ¹H-nuclear magnetic resonance (NMR) data of the obtained compound (96) are shown below.

¹H-NMR (CDCl₃) δ (ppm)=7.00 (s, 2H), 6.84 (d, 2H), 6.12 (dd, 2H), 5.76 (dd, 2H), 4.3 to 4.1 (m, 6H), 3.9 to 3.8 (m, 4H), 2.5 to 2.4 (m, 2H), 2.31 (s, 6H), 2.2 to 2.05 (m, 4H), 1.95 to 1.86 (m, 4H), 1.83 to 1.78 (m, 4H), 1.70 to 1.65 (m, 4H), 1.60 to 1.40 (m, 4H), 1.30 to 1.10 (m, 4H)

### Synthesis of Compound (12)

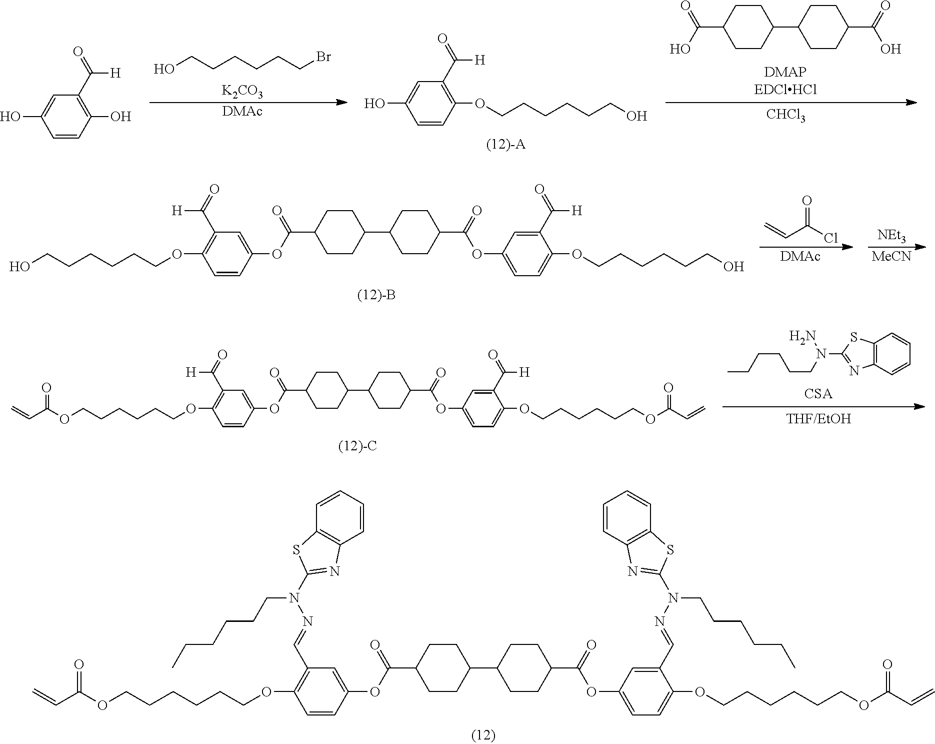

In a nitrogen atmosphere, 6.08 g of 2,5-hydroxybenzaldehyde and 80 mL of N,N-dimethylacetamide were added to a 300 mL three-necked flask, 6.0 mL of 6-bromo-1-hexanol was added dropwise thereto while stirring at room temperature, and 6.62 g of potassium was added to the mixture. After stirring at 80° C. for 4 hours, the mixture was cooled to room temperature, 100 mL of water and 100 mL of ethyl acetate were added thereto, and the mixture was stirred at room temperature. The organic layer recovered by liquid separation was concentrated, 100 mL of 1 M diluted hydrochloric acid and 100 mL of ethyl acetate were added thereto, and the mixture was stirred at room temperature. The organic layer recovered by liquid separation was concentrated and then purified by silica gel column chromatography (developing solvent: hexane/ethyl acetate) to obtain 3.26 g of a compound (12)-A represented by Formula (12)-A.

1.21 g of the compound (12)-A and 5 mL of chloroform were added to a 100 mL three-necked flask, 0.25 g of (trans,trans)-[1,1′-bicyclohexyl]-4,4′-dicarboxylic acid, 0.03 g of 4-dimethylaminopyridine, 1.17 g of 1-(3-dimethylaminopropyl)-3-ethylcarbodiimide hydrochloride, and 5 mL of chloroform were added thereto under stirring at room temperature, and the mixture was stirred at room temperature for 3 hours. The reaction solution was concentrated and purified by silica gel column chromatography (developing solvent: hexane/ethyl acetate) to obtain 0.61 g of a compound (12)-B represented by Formula (12)-B.

0.61 g of the compound (12)-B and 10 mL of N,N-dimethylacetamide were added to a 300 mL three-neck flask, and the mixture was stirred under ice cooling in a nitrogen atmosphere. 1.4 mL of acryloyl chloride was added dropwise thereto, the temperature was raised to room temperature, and the mixture was further stirred for 1 hour. 30 ml of water and 30 mL of ethyl acetate were added to this reaction solution, and the organic layer obtained by extraction and liquid separation was concentrated and then purified by silica gel column chromatography (developing solvent: hexane/ethyl acetate) and crystallization (chloroform/methanol) to obtain 0.30 g of a compound (12)-C represented by Formula (12)-C.

0.30 g of the compound (12)-C, 0.21 g of 2-(1-hexylhydrazino) benzothiazole, 0.12 g of (+)-10-camphorsulfonic acid, 6 mL of tetrahydrofuran, and 3 mL of ethanol was added to a 100 mL three-neck flask, and the mixture was stirred at 50° C. for 1 hour. The reaction solution was concentrated and purified by silica gel column chromatography (developing solvent: hexane/chloroform) and crystallization (chloroform/methanol) to obtain 0.43 g of a compound (12) represented by Formula (12).

The ¹H-NMR data of the obtained compound (12) are shown below.

¹H-NMR (CDCl₃) δ (ppm): 8.09 (s, 2H), 7.69-7.62 (m, 6H), 7.33 (dt, 2H), 7.14 (dt, 2H), 7.01 (dd, 2H), 6.89 (d, 2H), 6.40 (dd, 2H), 6.12 (dd, 2H), 5.82 (dd, 2H), 4.32 (t, 4H), 4.18 (t, 4H), 4.04 (t, 4H), 2.53 (tt, 2H), 2.24 (dd, 4H), 1.93 (d, 4H), 1.89 to 1.82 (m, 4H), 1.80 to 1.67 (m, 8H), 1.66 to 1.10 (m, 36H), 0.89 (t, 6H)

### Synthesis of Compound (93)

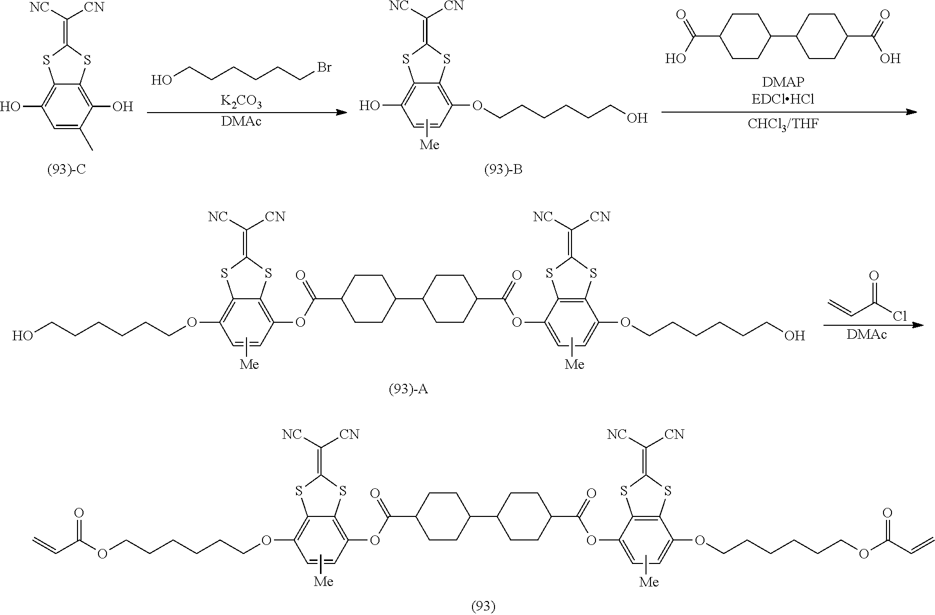

In a nitrogen atmosphere, 1.31 g of a raw material compound (93)-C represented by Formula (93)-C and 10 mL of N,N-dimethylacetamide were added to a 300 mL three-necked flask, 0.91 g of 6-bromo-1-hexanol was added dropwise thereto under stirring at room temperature, and 1.0 g of potassium carbonate was added to the mixture. After stirring at 80° C. for 4 hours, the mixture was cooled to room temperature, 100 ml of water and 100 mL of ethyl acetate were added thereto, and the mixture was stirred at room temperature. The organic layer recovered by liquid separation was concentrated, 100 mL of 1 M diluted hydrochloric acid and 100 mL of ethyl acetate were added thereto, and the mixture was stirred at room temperature. The organic layer recovered by liquid separation was concentrated and then purified by silica gel column chromatography (developing solvent: hexane/ethyl acetate) to obtain 0.50 g of a compound (93)-B represented by Formula (93)-B.

0.50 g of the compound (93)-B and 5 mL of tetrahydrofuran (THF) were added to a 100 mL three-neck flask, 0.15 g of (trans, trans)-[1,1′-bicyclohexyl]-4,4′-dicarboxylic acid, 0.01 g of 4-dimethylaminopyridine, 0.35 g of 1-(3-dimethylaminopropyl)-3-ethylcarbodiimide hydrochloride, and 5 mL of chloroform were added thereto under stirring at room temperature, and the mixture was stirred at room temperature for 12 hours. The reaction solution was concentrated and purified by silica gel column chromatography (developing solvent: hexane/ethyl acetate) to obtain 0.45 g of a compound (93)-A represented by Formula (93)-A.

0.45 g of the compound (93)-A and 5 mL of N,N-dimethylacetamide were added to a 100 mL three-neck flask, and the mixture was stirred under ice cooling in a nitrogen atmosphere. 0.3 mL of acryloyl chloride was added dropwise thereto, the temperature was raised to room temperature, and the mixture was further stirred for 1 hour. 10 ml of water and 20 mL of ethyl acetate were added to this reaction solution, and the organic layer obtained by extraction and liquid separation was concentrated and then purified by silica gel column chromatography (developing solvent: hexane/ethyl acetate) and crystallization (chloroform/methanol) to obtain 0.17 g of a compound (93) represented by Formula (93).

The ¹H-NMR data of the obtained compound (93) are shown below.

¹H-NMR (CDCl₃): δ (ppm)=7.00 (s, 2H), 6.84 (d, 2H), 6.10 (dd, 2H), 5.74 (dd, 2H), 4.3 to 4.1 (m, 6H), 3.9 to 3.8 (m, 4H), 2.5 to 2.4 (m, 4H), 2.30 (s, 6H), 2.2 to 2.05 (m, 6H), 1.95 to 1.86 (m, 4H), 1.83 to 1.78 (m, 6H), 1.70 to 1.65 (m, 4H), 1.60 to 1.40 (m, 6H), 1.30 to 1.15 (m, 6H)

### Synthesis of Compound (94)

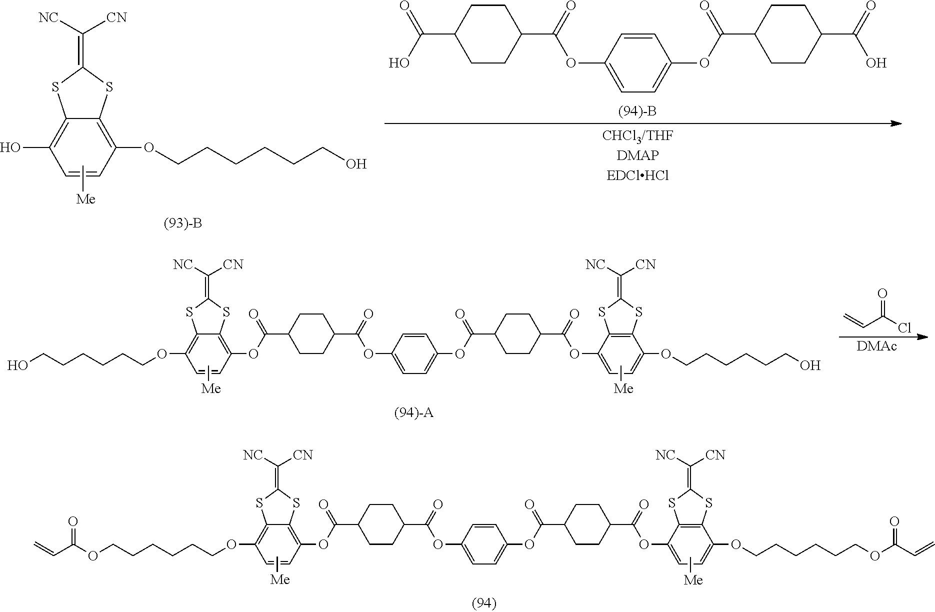

A raw material compound (94)-B represented by Formula (94)-B was synthesized in accordance with the synthesis method of the compound I-2-6 described in JP2018-35126A.

0.50 g of the compound (93)-B represented by Formula (93)-B and 5 mL of THF were added to a 100 mL three-neck flask, 0.26 g of the raw material compound (94)-B, 0.01 g of 4-dimethylaminopyridine, 0.30 g of 1-(3-dimethylaminopropyl)-3-ethylcarbodiimide hydrochloride, and 5 mL of chloroform were added thereto under stirring at room temperature, and the mixture was stirred at room temperature for 12 hours. The reaction solution was concentrated and purified by silica gel column chromatography (developing solvent: hexane/ethyl acetate) to obtain 0.36 g of a compound (94)-A represented by Formula (94)-A.

0.35 g of the compound (94)-A and 5 mL of N,N-dimethylacetamide were added to a 100 mL three-neck flask, and the mixture was stirred under ice cooling in a nitrogen atmosphere. 0.2 mL of acryloyl chloride was added dropwise thereto, the temperature was raised to room temperature, and the mixture was further stirred for 1 hour. 10 mL of water and 20 mL of ethyl acetate were added to this reaction solution, and the organic layer obtained by extraction and liquid separation was concentrated and then purified by silica gel column chromatography (developing solvent: hexane/ethyl acetate) and crystallization (chloroform/methanol) to obtain 0.13 g of a compound (94) represented by Formula (94).

The ¹H-NMR data of the obtained compound (94) are shown below.

¹H-NMR (CDCl₃): δ (ppm)=7.20 (s, 4H), 6.95 (s, 2H), 6.88 (d, 2H), 6.20 (dd, 2H), 5.78 (dd, 2H), 4.3 to 4.15 (m, 6H), 3.98 to 3.8 (m, 4H), 2.5 to 2.4 (m, 4H), 2.30 (s, 6H), 2.2 to 2.05 (m, 6H), 1.95 to 1.86 (m, 4H), 1.85 to 1.70 (m, 6H), 1.65 to 1.60 (m, 4H), 1.55 to 1.40 (m, 6H), 1.30 to 1.2 (m, 6H)

### Example 1

### Manufacture of Optical Film

### Synthesis of Monomer mA-1

4-Aminocyclohexanol (50.0 g), triethylamine (48.3 g), and N,N-dimethylacetamide (800 g) were weighed out into a 2 L three-neck flask comprising a stirring blade, a thermometer, a dropping funnel, and a reflux pipe, and stirred under ice cooling.

Next, methacrylic acid chloride (47.5 g) was added dropwise into the flask over 40 minutes using a dropping funnel, and after completion of the dropwise addition, the reaction solution was stirred at 40° C. for 2 hours.

The reaction solution was cooled to room temperature (23° C.) and then subjected to suction filtration to remove the precipitated salt. The obtained organic layer was transferred to a 2 L three-neck flask comprising a stirring blade, a thermometer, a dropping funnel, and a reflux pipe, and stirred under water cooling.

Next, N,N-dimethylaminopyridine (10.6 g) and triethylamine (65.9 g) were added to the flask, 4-n-octyloxycinnamic acid chloride (127.9 g) dissolved in advance in tetrahydrofuran (125 g) was added dropwise into the flask over 30 minutes using a dropping funnel, and after completion of the dropwise addition, the reaction solution was stirred at 50° C. for 6 hours. The reaction solution was cooled to room temperature, and then subjected to liquid separation and washed with water, the obtained organic layer was dried over anhydrous magnesium sulfate, and the obtained solution was concentrated to obtain a yellowish white solid.

The obtained yellowish white solid was dissolved in methyl ethyl ketone (400 g) by heating and recrystallized to obtain 76 g of a monomer mA-1 shown below as a white solid (yield 40%).

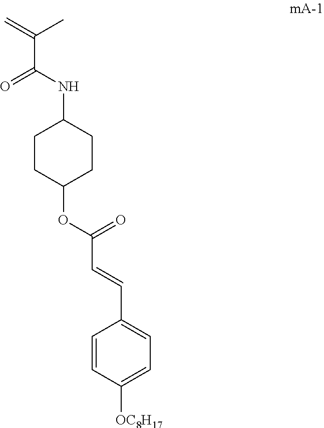

### Other Monomers

As the following monomer mB-1 forming a repeating unit B-1, CYCLOMER M-100 (manufactured by Daicel Corporation) was used.

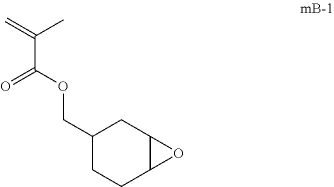

### Synthesis of Polymer P-1

A flask comprising a cooling pipe, a thermometer, and a stirrer was charged with 2-butanone (5 parts by mass) as a solvent, and while flowing nitrogen in the flask at 5 mL/min, the resultant was refluxed by heating in a water bath. A solution obtained by mixing the monomer mA-1 (1.2 parts by mass), the monomer mB-1 (8.8 parts by mass), 2,2′-azobis (isobutyronitrile) (1 part by mass) as a polymerization initiator, and 2-butanone (5 parts by mass) as a solvent was added dropwise thereto over 3 hours, and the obtained reaction solution was stirred while maintaining the reflux state for another 3 hours. After completion of the reaction, the reaction solution was allowed to cool to room temperature, and 2-butanone (30 parts by mass) was added to the reaction solution and diluted to obtain a polymer solution having a polymer concentration of about 20% by mass. The obtained polymer solution was poured into a large excess of methanol to precipitate the polymer, the precipitate was separated by filtration, and the obtained solid content was washed with a large amount of methanol and then blast-dried at 50° C. for 12 hours to obtain a polymer P-1 having a photoalignment group.

### Preparation of Composition for Photoalignment Film

A composition 1 for a photoalignment film was prepared as follows.

Composition 1 for photoalignment film

| | |
|---|---|
| Polymer P-1 | 100.00 parts by mass |
| The following thermal acid generator D-1 | 3.00 parts by mass |
| Diisopropylethylamine | 0.60 parts by mass |
| Butyl acetate | 953.12 parts by mass |
| Methyl ethyl ketone | 238.28 parts by mass |

Thermal acid generator D-1

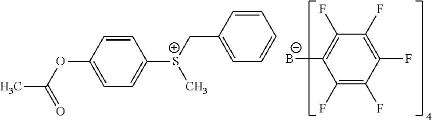

A composition 1 for forming a photoalignment film was applied onto glass by spin coating. Thereafter, the glass having the composition 1 for a photoalignment film applied thereon was dried on a hot plate at 140° C. for 1 minute to remove the solvent, thereby forming a precursor film having a thickness of 0.3 μm. The obtained precursor film was irradiated with polarized ultraviolet rays (8 mJ/cm², using an ultra-high-pressure mercury lamp) to form a photoalignment film.

Next, the following polymerizable composition was applied onto the photoalignment film by spin coating.

The coating film was subjected to an alignment treatment at a temperature shown in Table 4 to form a liquid crystal layer.

Thereafter, the liquid crystal layer was cooled to the temperature at the time of exposure shown in Table 4 below, and subjected to alignment fixation by irradiation with ultraviolet rays at 300 mJ/cm² to form an optically anisotropic film, thereby manufacturing an optical film.

Polymerizable Composition

| | |
|---|---|
| The following compound (3) | 43.00 parts by mass |
| The following liquid crystal compound (I-3-1) | 57.00 parts by mass |
| The following polymerization initiator S-1 (oxime-type) | 0.50 parts by mass |
| The following fluorine-containing compound A | 0.15 parts by mass |
| Chloroform | 570.35 parts by mass |

Compound (3) 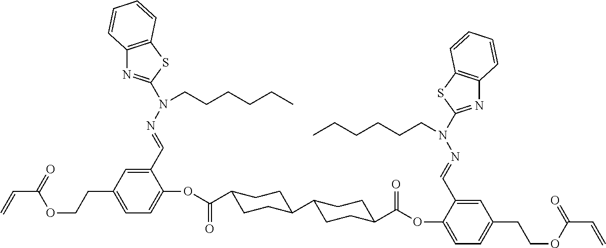 [SMILES: C=CC(=O)OCCc1ccc(OC(=O)C2CCC(C3CCC(C(=O)Oc4ccc(CCOC(=O)C=C)cc4/C=N/N(CCCCCC)c4nc5ccccc5s4)CC3)CC2)c(/C=N/N(CCCCCC)c2nc3ccccc3s2)c1]
Liquid crystal compound (I-3-1) 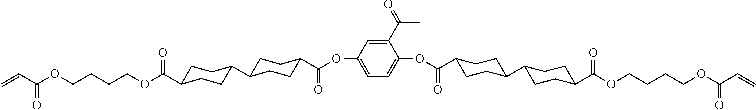 [SMILES: C=CC(=O)OCCCCOC(=O)C1CCC(C2CCC(C(=O)Oc3ccc(OC(=O)C4CCC(C5CCC(C(=O)OCCCCOC(=O)C=C)CC5)CC4)c(C(C)=O)c3)CC2)CC1]
Polymerization initiator S-1 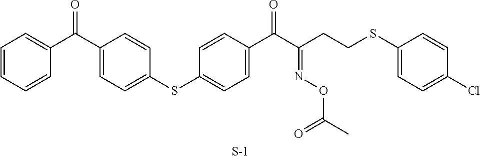
Fluorine-containing compound A

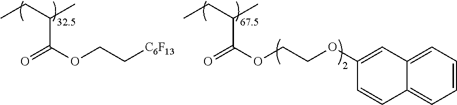

### Examples 2 to 20

An optical film was manufactured in the same manner as in Example 1, except that the compound was changed to the compound shown in Table 4 below.

### Comparative Examples 1 to 3

An optical film was manufactured in the same manner as in Example 1, except that the compound was changed to the compound shown in Table 4 below.

### Retardation

With regard to the manufactured optical film, a retardation value at a wavelength of 450 nm (Re(450)) and a retardation value at a wavelength of 550 nm (Re(550)) were measured using AxoScan (OPMF-1, manufactured by Opto Science, Inc.), and Re (450)/Re(550) was calculated. The results are shown in Table 4 below.

### Moisture-Heat Resistance

With regard to test conditions for the moisture-heat resistance, a test in which an object was left to stand in an environment of 100° C. and a relative humidity of 95% for 72 hours was carried out.

The Re(550) of the optical film before the test and the Re(550) of the optical film after the test were measured, and the moisture-heat resistance was evaluated according to the following standard. The results are shown in Table 4 below.
A: A change amount of Re(550) after the test with respect to Re(550) before the test was less than 10% of Re(550) before the test
B: The change amount of Re(550) after the test with respect to Re(550) before the test was 10% or more and less than 30% of Re(550) before the test
C: The change amount of Re (550) after the test with respect to Re(550) before the test was 30% or more of Re(550) before the test

### Light Resistance

The manufactured optical film was subjected to a test in which a glass substrate was set in a xenon irradiation machine (SX75 manufactured by Suga Test Instruments Co., Ltd.) so that the coating film of the polymerizable composition was an irradiation surface, and irradiated for 200 hours using a #275 filter.

The Re(550) of the optical film before the test and the Re(550) of the optical film after the test were measured, and the light resistance was evaluated according to the following standards. The results are shown in Table 4 below.
A: A change amount of Re(550) after the test with respect to Re(550) before the test was less than 5% of Re(550) before the test
B: The change amount of Re(550) after the test with respect to Re(550) before the test was 5% or more and less than 15% of Re(550) before the test
C: The change amount of Re(550) after the test with respect to Re(550) before the test was 15% or more of Re(550) before the test

TABLE 4

| | | | Liquid crystal | | | | | | |
|---|---|---|---|---|---|---|---|---|---|
| | Compound represented | | compound | | | | | | |
| | by Formula (I-1) and | | represented by | | Alignment | | | | |
| | the like | | Formula (I-2) | | treatment | Exposure | | | |
| | | Parts by | | Parts by | temperature | temperature | Re(450)/ | Moisture-heat | Light |
| | Type | mass | Type | mass | (° C.) | (° C.) | Re(550) | resistance | resistance |
| Example 1 | (3) | 43 | I-3-1 | 57 | 200 | 120 | 0.81 | A | A |
| Example 2 | (12) | 43 | I-3-1 | 57 | 200 | 120 | 0.81 | A | A |
| Example 3 | (78) | 43 | I-3-1 | 57 | 200 | 200 | 0.81 | A | A |
| Example 4 | (93) | 43 | I-3-1 | 57 | 200 | 200 | 0.81 | A | A |
| Example 5 | (94) | 50 | I-3-1 | 50 | 200 | 120 | 0.81 | A | A |
| Example 6 | (96) | 40 | I-3-1 | 60 | 200 | 120 | 0.81 | A | A |
| Example 7 | (98) | 43 | I-3-1 | 57 | 200 | 200 | 0.88 | A | B |
| Example 8 | (104) | 43 | I-3-1 | 57 | 200 | 200 | 0.88 | A | B |
| Example 9 | (105) | 43 | I-3-1 | 57 | 200 | 200 | 0.81 | A | B |
| Example 10 | (106) | 43 | I-3-1 | 57 | 200 | 200 | 0.91 | A | B |
| Example 11 | (108) | 43 | I-3-1 | 57 | 200 | 200 | 0.81 | A | B |
| Example 12 | (111) | 43 | I-3-1 | 57 | 200 | 200 | 0.84 | A | B |
| Example 13 | (112) | 43 | I-3-1 | 57 | 200 | 200 | 0.81 | A | A |
| Example 14 | (119) | 43 | I-3-1 | 57 | 200 | 200 | 0.88 | A | A |
| Example 15 | (126) | 43 | I-3-1 | 57 | 200 | 200 | 0.95 | A | A |
| Example 16 | (133) | 43 | I-3-1 | 57 | 200 | 200 | 0.81 | A | B |
| Example 17 | (137) | 43 | I-3-1 | 57 | 200 | 200 | 0.84 | A | A |
| Example 18 | (140) | 43 | I-3-1 | 57 | 200 | 200 | 0.81 | A | A |
| Example 19 | (155) | 30 | I-3-1 | 70 | 200 | 200 | 0.81 | A | A |
| Example 20 | (157) | 43 | I-3-1 | 57 | 200 | 200 | 0.81 | A | A |
| Comparative | (201) | 43 | I-3-1 | 57 | 200 | 120 | 0.93 | B | B |
| Example 1 | | | | | | | | | |
| Comparative | (202) | 43 | I-3-1 | 57 | 200 | 120 | 0.84 | C | B |
| Example 2 | | | | | | | | | |
| Comparative | (203) | 43 | I-3-1 | 57 | 200 | 120 | 0.95 | B | B |
| Example 3 | | | | | | | | | |

The structures of the compounds in Table 4 are as shown below.

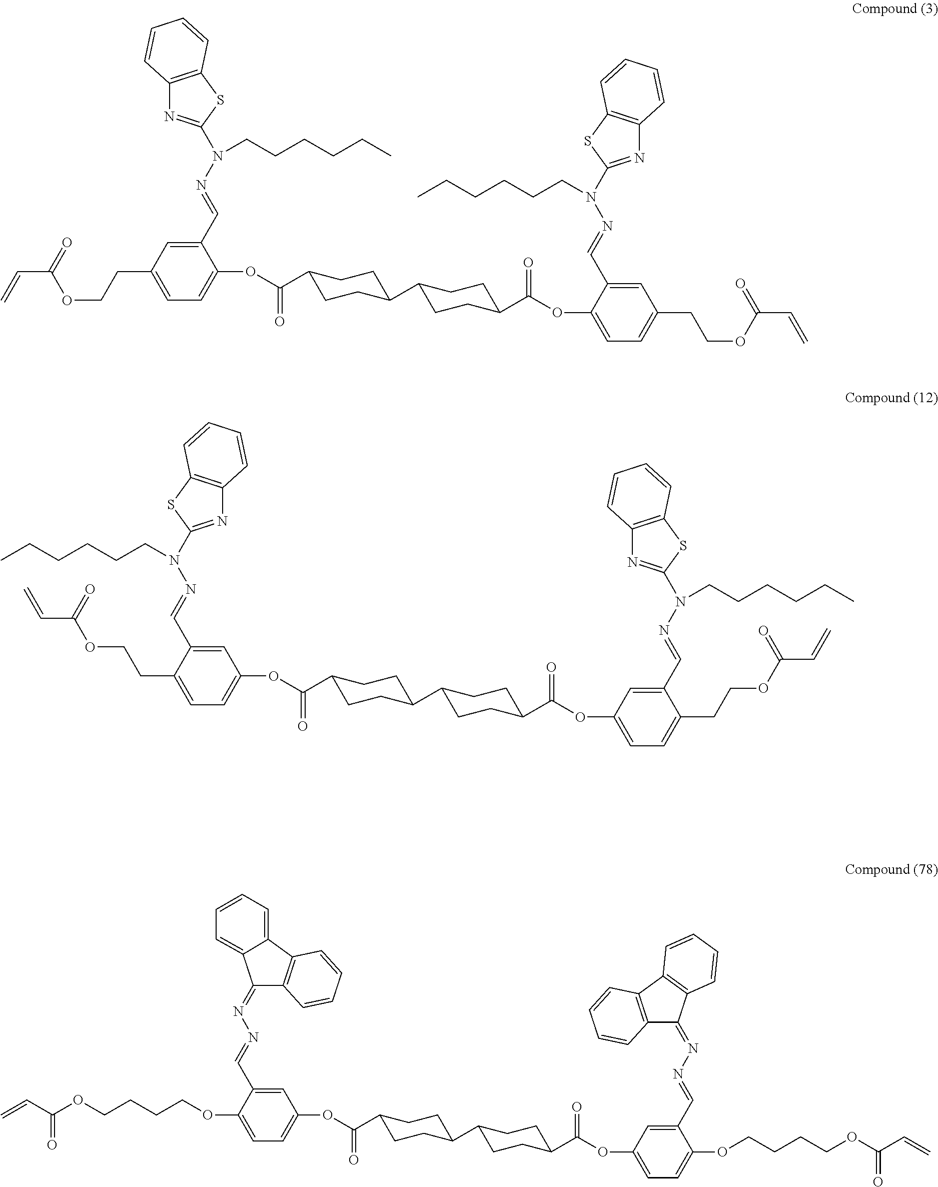

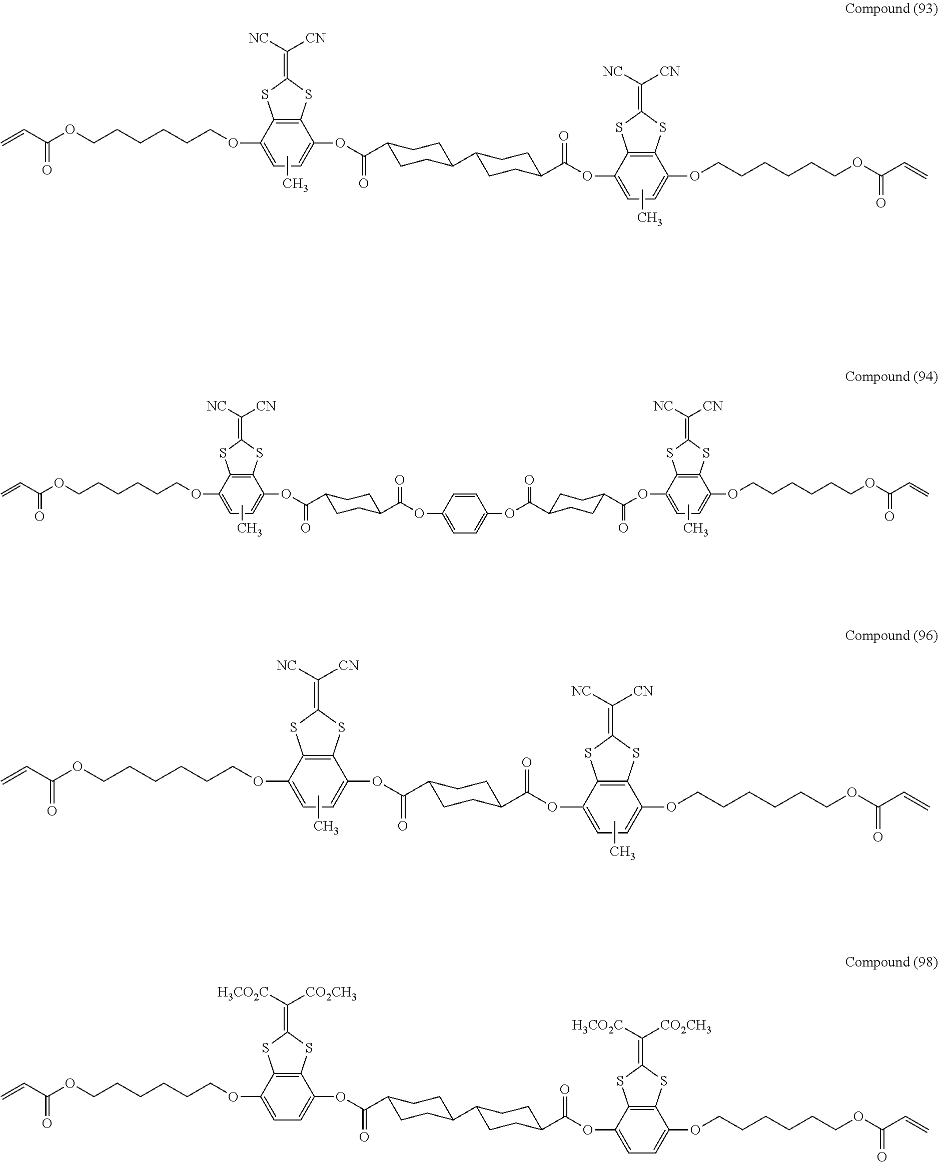

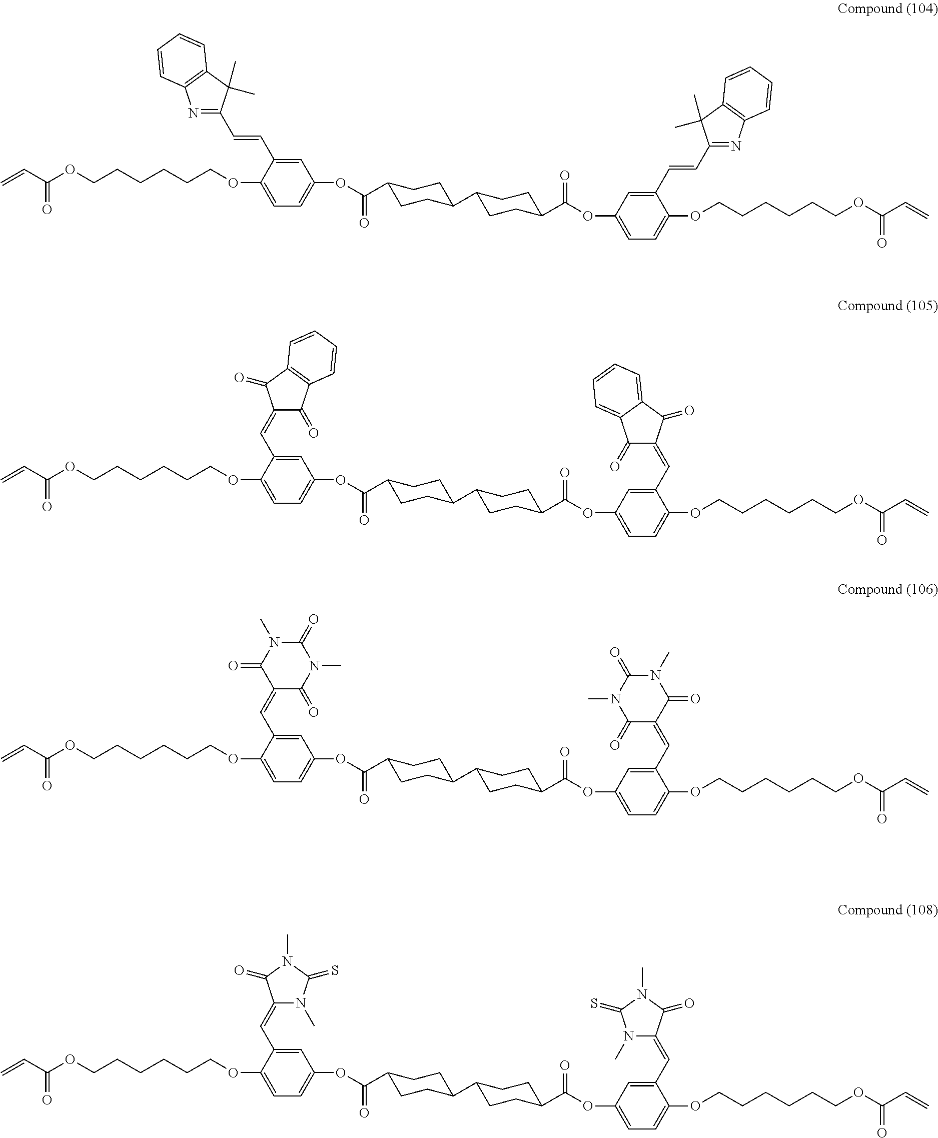

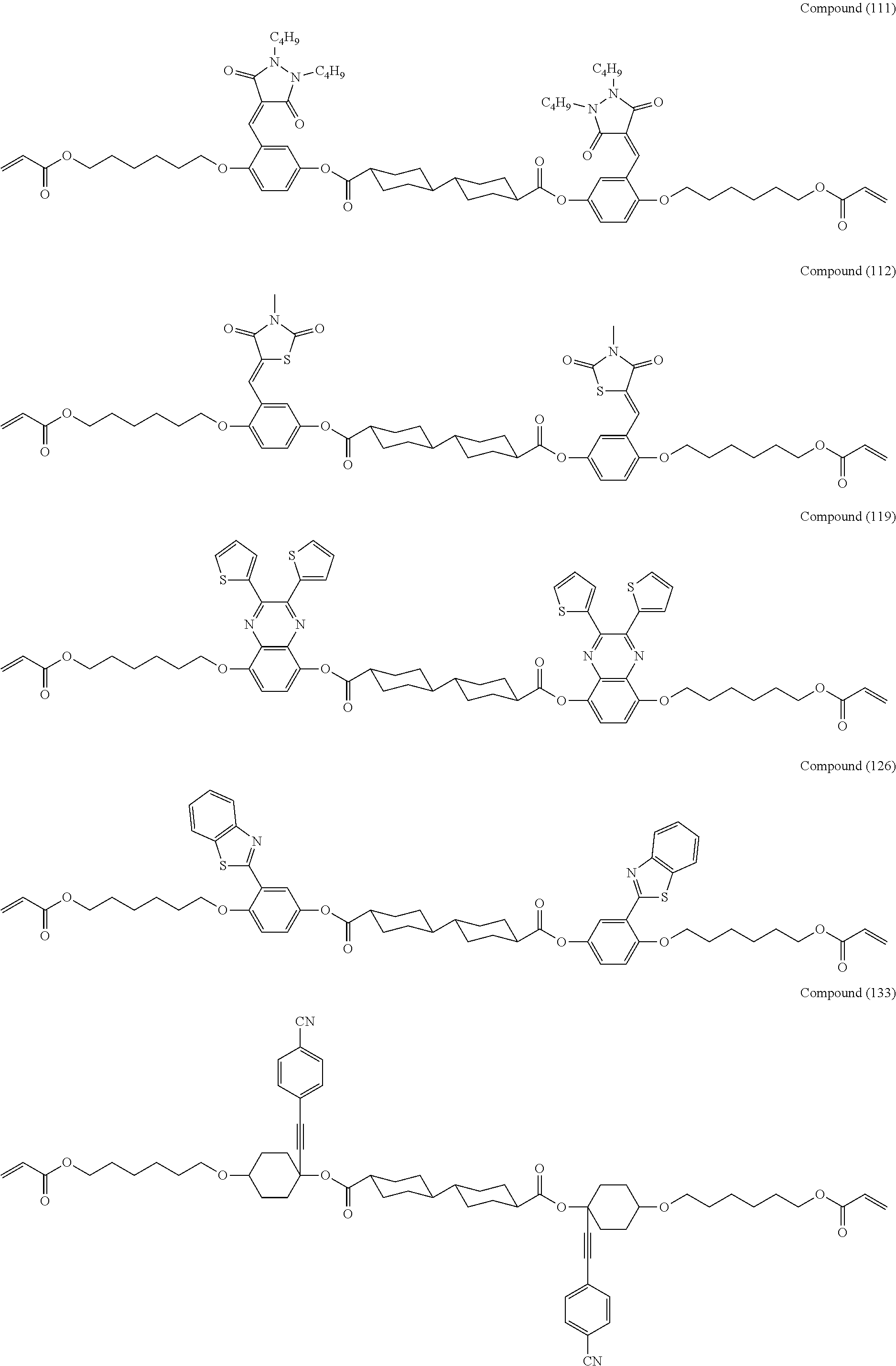

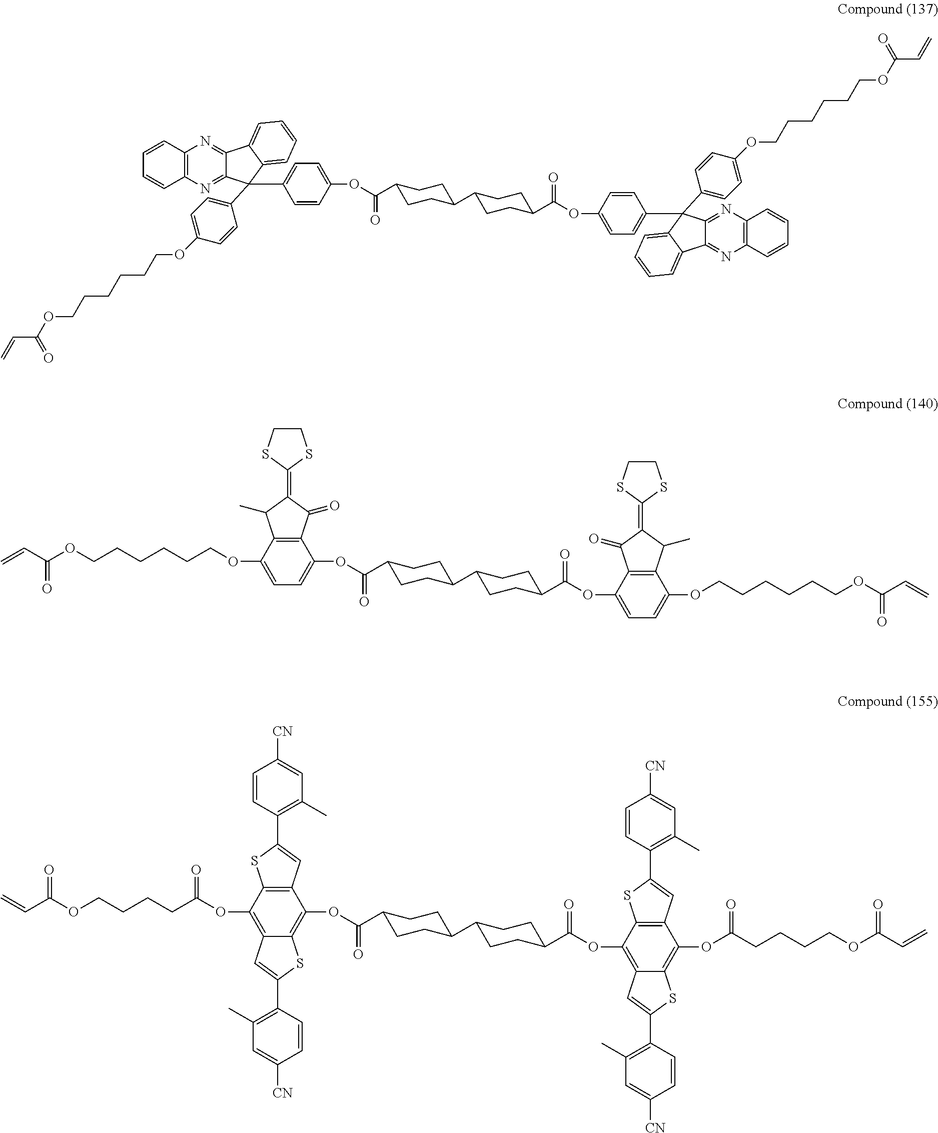

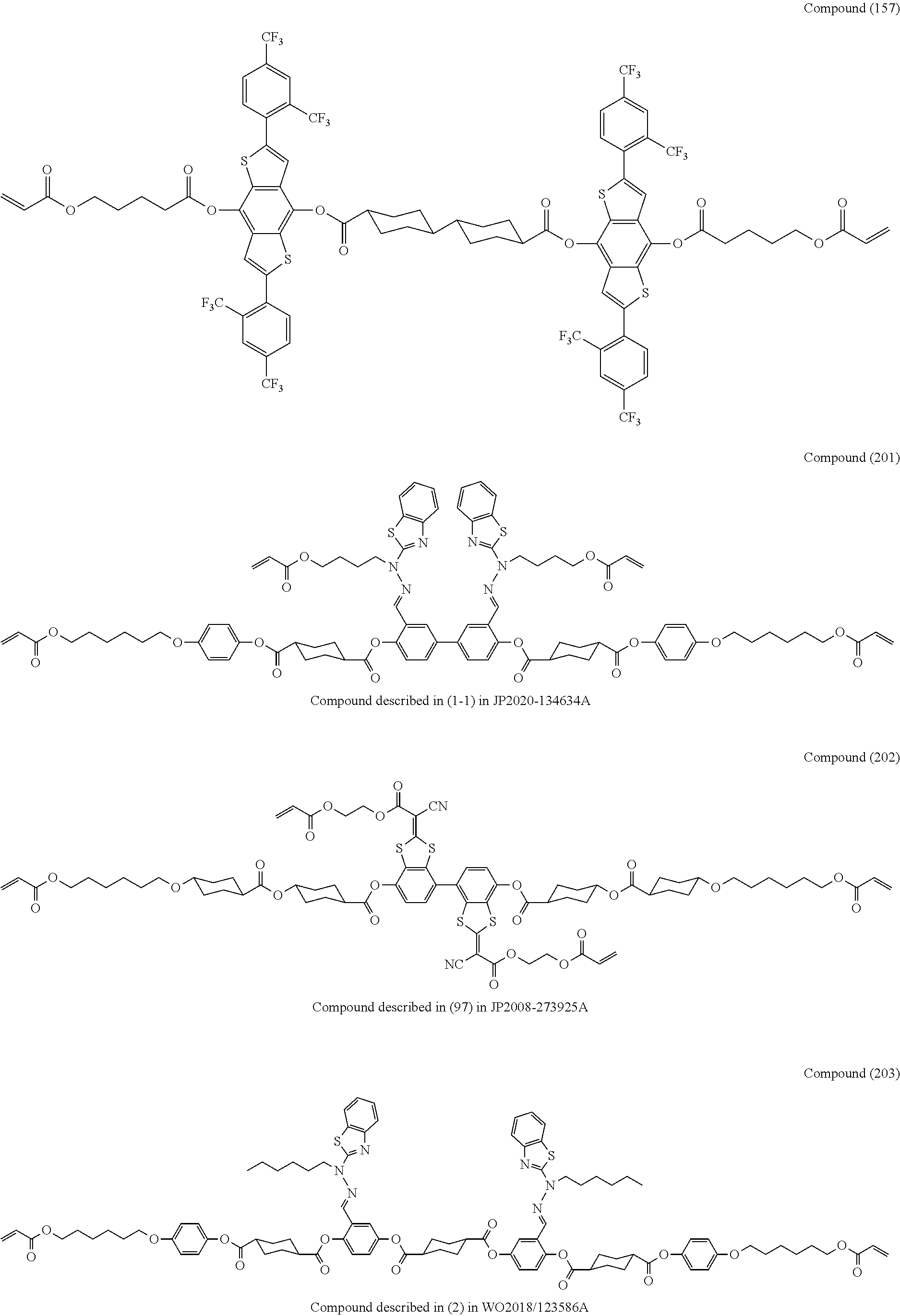

From the results shown in Table 4 above, it was found that in a case where the compound represented by Formula (I-1) is not blended, the reverse wavelength dispersibility of an optically anisotropic film thus formed is low, and further, any of the moisture-heat resistance and light resistance is poor (Comparative Examples 1 to 3).

On the contrary, it was found that in a case where the compound represented by Formula (I-1) is blended, an optically anisotropic film having excellent reverse wavelength dispersibility, moisture-heat resistance, and light resistance can be formed (Examples 1 to 20).

### EXPLANATION OF REFERENCES

10: optical film
12: optically anisotropic film
14: alignment film
16: support
18: hard coat layer

What is claimed is:

1. A compound represented by Formula (I-1),
P¹-SP¹-Ar¹-Mes-Ar²-SP²-P² (I-1)
in Formula (I-1),
P¹ and P² each independently represent a monovalent organic group,
SP¹ and SP² each independently represent a linear or branched alkylene group having 1 to 12 carbon atoms, or a divalent linking group in which one or more of —CH₂—'s constituting the linear or branched alkylene group having 1 to 12 carbon atoms are substituted with —O—, —S—, —NH—, —N(Q)—, or —CO—, where Q represents a substituent,
Mes represents a group represented by Formula (I-2), and
Ar¹ and Ar² each independently represent any ring structure selected from the group consisting of groups represented by Formulae (Ar-1) to (Ar-15), 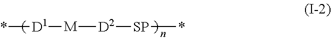
in Formula (I-2),
* represents a bonding position to Ar¹ or Ar²,
M represents a 1,4-phenylene group, a 1,4-cyclohexylene group, a pyridine-2,5-diyl group, a pyrimidine-2,5-diyl group, a naphthalene-2,6-diyl group, a naphthalene-1,4-diyl group, a tetrahydronaphthalene-2,6-diyl group, a decahydronaphthalene-2,6-diyl group, or a 1,3-dioxane-2,5-diyl group, provided that a hydrogen atom contained in these groups may be substituted with a fluorine atom, a chlorine atom, a bromine atom, an iodine atom, a pentafluorosulfuranyl group, a cyano group, a nitro group, an isocyano group, a thioisocyano group, or an alkyl group having 1 to 20 carbon atoms,
D¹ and D² each independently represent —O—, —S—, —OCH₂—, —CH₂CH₂—, —CO—, —COO—, —OCO—, —CO—S—, —OCO—O—, —CO—NH—, —NH—CO—, —SCH₂—, —CF₂O—, —CF₂S—, —CH═CH—COO—, —CH═CH—OCO—, —COO—CH₂CH₂—, —OCO—CH₂CH₂—, —COO—CH₂—, —OCO—CH₂—, —CH═CH—, —N═N—, —CH═N—, —CH═N—N═CH—, —CF═CF—, —C═C—, or a single bond,
SP represents a single bond, a linear or branched alkylene group having 1 to 12 carbon atoms, or a divalent linking group in which one or more of —CH₂—'s constituting the linear or branched alkylene group having 1 to 12 carbon atoms are substituted with —O—, —S—, —NH—, —N(Q)—, or —CO—, where Q represents a substituent, and
n represents an integer of 1 to 5, provided that in a case where n represents an integer of 2 to 5, a plurality of M's may be the same as or different from each other, a plurality of D¹'s may be the same as or different from each other, a plurality of D²'s may be the same as or different from each other, and a plurality of SP's may be the same as or different from each other, 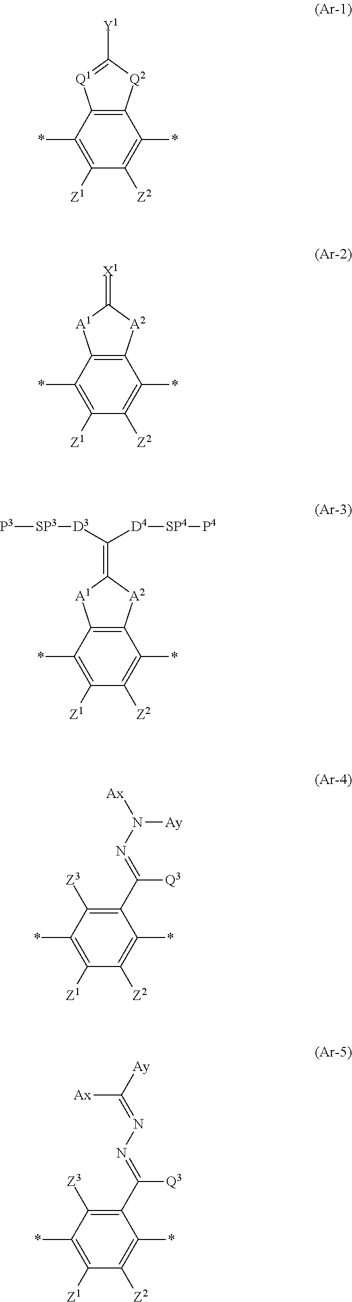 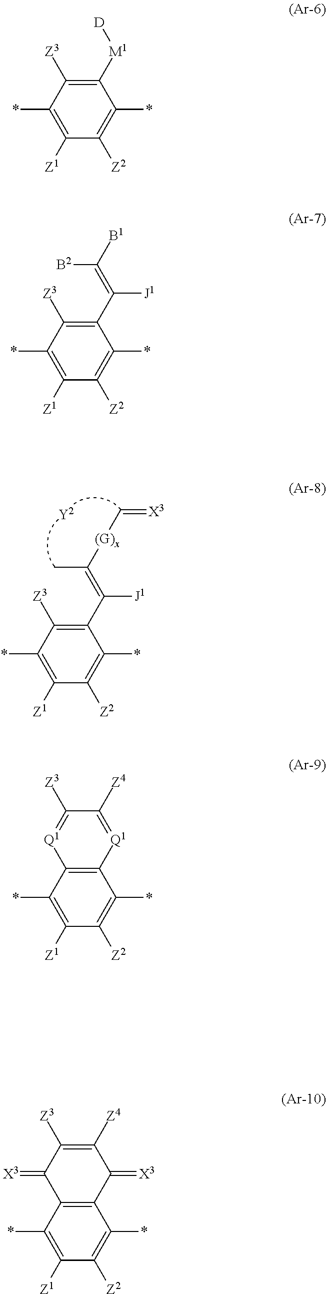 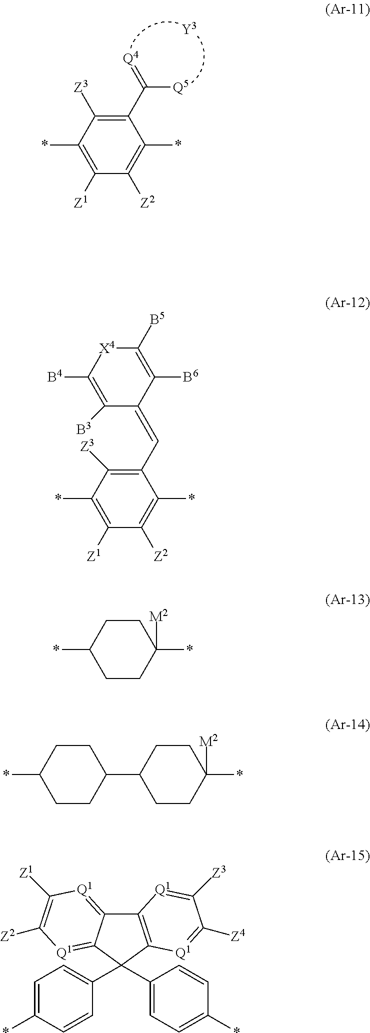
in Formulae (Ar-1) to (Ar-15),
* represents a bonding position to Mes or SP¹ for Ar¹, and represents a bonding position to Mes or SP² for Ar²,
Q¹ represents N or CH,
Q² represents —S—, —O—, or —N(R⁶)—, where R⁶ represents a hydrogen atom or an alkyl group having 1 to 6 carbon atoms,
Y¹ represents an alkynyl group having 2 to 16 carbon atoms, an aromatic hydrocarbon group having 6 to 12 carbon atoms, or an aromatic heterocyclic group having 3 to 12 carbon atoms, each of which may have a substituent,
Z¹, Z², Z³, and Z⁴ each independently represent a hydrogen atom, a monovalent aliphatic hydrocarbon group having 1 to 20 carbon atoms, a monovalent alicyclic hydrocarbon group having 3 to 20 carbon atoms, a monovalent aromatic group having 6 to 18 π electrons, a halogen atom, a cyano group, a nitro group, —OR⁷, —NR⁸R⁹, —SR¹⁰, —COOR¹¹, or —COR¹², where R⁷ to R¹² each independently represent a hydrogen atom or an alkyl group having 1 to 6 carbon atoms, provided that Z¹ and Z², or Z³ and Z⁴ may be bonded to each other to form an aromatic ring,
A¹ and A² each independently represent a group selected from the group consisting of —CW¹W²—, —O—, —N(R¹³)—, —S—, and —CO—, where W¹ and W² each independently represent a hydrogen atom, a halogen atom, or an alkyl group having 1 to 6 carbon atoms, and R¹³ represents a hydrogen atom or a substituent,
X¹ represents a non-metal atom of Groups 14 to 16, provided that a hydrogen atom or a substituent may be bonded to the non-metal atom,
D³ and D⁴ each independently represent a single bond, or a divalent linking group consisting of —CO—, —O—, —S—, —C(═S)—, —CR¹R²—, —CR³═CR⁴—, —NR⁵—, or a combination of two or more of these groups, where R¹ to R⁵ each independently represent a hydrogen atom, a fluorine atom, or an alkyl group having 1 to 4 carbon atoms,
SP³ and SP⁴ each independently represent a single bond, a linear or branched alkylene group having 1 to 12 carbon atoms, or a divalent linking group in which one or more of —CH₂—'s constituting the linear or branched alkylene group having 1 to 12 carbon atoms are substituted with —O—, —S—, —NH—, —N(Q)—, or —CO—, where Q represents a substituent,
P³ and P⁴ each independently represent a monovalent organic group,
Ax represents an organic group having 2 to 30 carbon atoms, which has at least one aromatic ring selected from the group consisting of an aromatic hydrocarbon ring and an aromatic heterocyclic ring,
Ay represents a hydrogen atom, an alkyl group having 1 to 12 carbon atoms, which may have a substituent, or an organic group having 2 to 30 carbon atoms, which has at least one aromatic ring selected from the group consisting of an aromatic hydrocarbon ring and an aromatic heterocyclic ring,
the aromatic ring in each of Ax and Ay may have a substituent, and Ax and Ay may be bonded to each other to form a ring,
Q³ represents a hydrogen atom or an alkyl group having 1 to 12 carbon atoms, which may have a substituent,
M¹ represents —NR¹⁴—, —CH═CH—, —N═CH—, —CH═N—, —N═N—, or —C═C—, where R¹⁴ represents a hydrogen atom or an alkyl group having 1 to 6 carbon atoms,
D represents a group represented by any of Formulae (D-1) to (D-10) which may have a substituent,
J¹ represents a hydrogen atom or an alkyl group,
B¹ represents —C(═X²)—B¹¹ or —CN, provided that B¹¹ represents a substituent and X² represents ═O, ═S, ═NR¹⁵, or ═C(CN)₂, where R¹⁵ represents a substituent,
B² represents a hydrogen atom or a substituent,
Y² represents an atomic group necessary to form a carbon ring or a heterocyclic ring,
G represents an atomic group necessary to complete a conjugated double bond chain, x represents 0 or 1,
X³ represents ═O, ═S, ═NR¹⁵, or ═C(CN)₂, where R¹⁵ represents a substituent,
Q⁴ represents ═N— or ═CR¹⁶—, where R¹⁶ represents a hydrogen atom or a substituent,
Q⁵ represents a non-metal atom of Groups 14 to 16, provided that a hydrogen atom or a substituent may be bonded to the non-metal atom,
Y³ represents an atomic group necessary to form a carbon ring or a heterocyclic ring with Q⁴ and Q⁵,
B³, B⁴, B⁵, and B⁶ each independently represent a hydrogen atom or a substituent,
X⁴ represents —O—, —S—, or —NR¹⁷—, where R¹⁷ represents a hydrogen atom or a substituent, and
M² represents an alkynyl group, 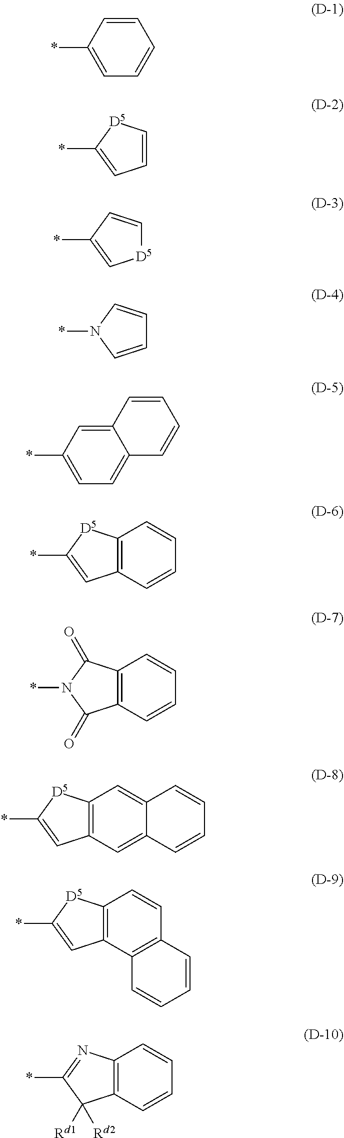
in Formulae (D-1) to (D-10),
* represents a bonding position to M¹,
D⁵ represents —O—, —S—, or —NR^{D1}—, where R^{D1} represents a hydrogen atom, an alkyl group having 1 to 5 carbon atoms, an alkanoyl group having 1 to 5 carbon atoms, or a phenyl group which may have a substituent,
R^{d1} and R^{d2} each independently represent an alkyl group having 1 to 20 carbon atoms, which may have a substituent, provided that one —CH₂— or two or more —CH₂—'s which are not adjacent to each other, constituting the alkyl group, may be each independently substituted with —O—, —S—, —CO—, —COO—, —OCO—, —CO—S—, —S—CO—, —SO₂—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C═C—, and R^{d1} and R^{d2} may be bonded to each other to form a 3- to 7-membered non-aromatic hydrocarbon ring, the non-aromatic hydrocarbon ring may have a substituent, and a carbon atom constituting the non-aromatic hydrocarbon ring may be substituted with a heteroatom, and
—CH═ constituting a ring structure in each of Formulae (D-1) to (D-10) may be substituted with —N═.

2. A polymerizable composition comprising:
the compound represented by Formula (I-1) according to claim 1; and
a liquid crystal compound represented by Formula (II),
wherein a content of the compound represented by Formula (I-1) is 10% by mass or more with respect to a total mass of the compound represented by Formula (I-1) and the liquid crystal compound represented by Formula (II),
P¹¹-L¹-D¹⁵-(A¹¹)ₐ₁-D¹³-(G¹)_{g1}-D¹¹-[Ar³-D¹²]_{q1}-(G²)_{g2}-D¹⁴-(A¹²)ₐ₂-D¹⁶-L²-P¹² (II)
in Formula (II),
a1, a2, g1, and g2 each independently represent 0 or 1, provided that at least one of a1 or g1 represents 1, and at least one of a2 or g2 represents 1,
q1 represents 1 or 2,
D¹¹, D¹², D¹³, D¹⁴, D¹⁵, and D¹⁶ each independently represent a single bond, or a divalent linking group consisting of —CO—, —O—, —S—, —C(═S)—, —CR¹R²—, —CR³═CR⁴—, —NR⁵—, or a combination of two or more of these groups, where R¹ to R⁵ each independently represent a hydrogen atom, a fluorine atom, or an alkyl group having 1 to 12 carbon atoms, provided that in a case where q1 is 2, a plurality of D¹²'s may be the same as or different from each other,
G¹ and G² each independently represent an aromatic ring having 6 to 20 carbon atoms, which may have a substituent, or a divalent alicyclic hydrocarbon group having 5 to 20 carbon atoms, which may have a substituent, where one or more of —CH₂—'s constituting the alicyclic hydrocarbon group may be substituted with —O—, —S—, or —NH—,
A¹¹ and A¹² each independently represent an aromatic ring having 6 to 20 carbon atoms, which may have a substituent, or a divalent alicyclic hydrocarbon group having 5 to 20 carbon atoms, which may have a substituent, where one or more of —CH₂—'s constituting the alicyclic hydrocarbon group may be substituted with —O—, —S—, or —NH—,
L¹ and L² each independently represent a single bond, a linear or branched alkylene group having 1 to 14 carbon atoms, or a divalent linking group in which one or more of —CH₂—'s constituting the linear or branched alkylene group having 1 to 14 carbon atoms are substituted with —O—, —S—, —NH—,—N(Q)—, or —CO—, where Q represents a substituent,
P¹¹ and P¹² each independently represent a monovalent organic group, where at least one of P¹¹ or P¹² represents a polymerizable group, and
Ar³ represents an aromatic ring having 6 to 20 carbon atoms, which may have a substituent, or a divalent alicyclic hydrocarbon group having 5 to 20 carbon atoms, which may have a substituent, where one or more of —CH₂—'s constituting the alicyclic hydrocarbon group may be substituted with —O—, —S—, or —NH—, provided that in a case where q1 is 2, a plurality of Ar³'s may be the same as or different from each other.

3. An optically anisotropic film obtained by polymerizing the polymerizable composition according to claim 2.

4. An optical film comprising:
the optically anisotropic film according to claim 3.

5. A polarizing plate comprising:
the optical film according to claim 4; and
a polarizer.

6. An image display device comprising:
the optical film according to claim 4.

7. An image display device comprising:
the polarizing plate according to claim 5.
